(19) United States
(12) Patent Application Publication
(10) Pub. No.: US 2010/0222349 A1
(43) Pub. Date: Sep. 2, 2010

(54) QUINOLINE DERIVATIVES USED TO TREAT INFLAMMATORY AND ALLERGIC DISEASES

(75) Inventors: Paul Martin Gore, Hertfordshire (GB); Ashley Paul Hancock, Hertfordshire (GB); Simon Teanby Hodgson, Hertfordshire (GB); Michael Joachim Kranz, Hertfordshire (GB); Panayiotis Alexandrou Procopiou, Hertfordshire (GB); Sadie Vile, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford, Middlesex (GB)

(21) Appl. No.: 12/681,968

(22) PCT Filed: Oct. 15, 2008
(86) PCT No.: PCT/EP08/63881

§ 371 Date: Apr. 7, 2010

Related U.S. Application Data
(60) Provisional application No. 60/980,222, filed on Oct. 16, 2007.

(51) Int. Cl. A61K 31/5377; C07D 401/14; A61K 31/4709; C07D 413/14; A61P 37/08; A61P 11/06

(52) U.S. Cl. 514/235.2; 546/177; 514/314; 544/128

(57) ABSTRACT

The present invention relates to compounds of formula (I)

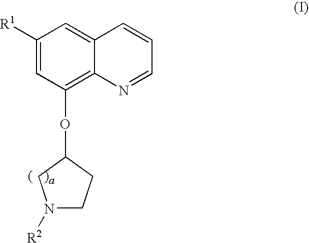

and salts thereof, processes for their preparation, to compositions containing them and to their use in the treatment of various diseases, such as allergic rhinitis.

The present invention relates to a class of compounds which are quinolinyloxypiperidine and pyrrolidine derivatives, processes for their preparation, pharmaceutical compositions containing them and to their use in the treatment of various inflammatory and/or allergic diseases, in particular inflammatory and/or allergic diseases of the respiratory tract.

Allergic rhinitis (seasonal and perennial), pulmonary inflammation and congestion are medical conditions that are often associated with other conditions such as asthma and chronic obstructive pulmonary disease (COPD). In general, these conditions are mediated, at least in part, by inflammation associated with the release of histamine from various cells, in particular mast cells.

Allergic rhinitis, which includes ‘hay fever’ affects a large proportion of the population worldwide. There are two types of allergic rhinitis, seasonal and perennial. The clinical symptoms of seasonal allergic rhinitis typically include nasal itching and irritation, sneezing and watery rhinorrhea, which is often accompanied by nasal congestion. The clinical symptoms of perennial allergic rhinitis are similar, except that nasal blockage may be more pronounced. Either type of allergic rhinitis may also cause other symptoms, such as itching of the throat and/or eyes, epiphora and oedema around the eyes. The symptoms of allergic rhinitis may vary in intensity from the nuisance level to debilitating.

Allergic rhinitis and other allergic conditions are associated with the release of histamine from various cell types, but particularly mast cells. The physiological effects of histamine are classically mediated by three receptor subtypes, termed H1, H2 and H3. H1 receptors are widely distributed throughout the CNS and periphery, and are involved in wakefulness and acute inflammation. H2 receptors mediate gastric acid secretion in response to histamine. H3 receptors are present on the nerve endings in both the CNS and periphery and mediate inhibition of neurotransmitter release [Hill et al., Pharmacol. Rev., 49:253-278, (1997)]. Recently a fourth member of the histamine receptor family has been identified, termed the H4 receptor [Hough, Mol. Pharmacol., 59:415-419, (2001)]. Whilst the distribution of the H4 receptor appears to be restricted to cells of the immune and inflammatory systems, a physiological role for this receptor remains to be identified.

The activation of H1 receptors in blood vessels and nerve endings are responsible for many of the symptoms of allergic rhinitis, which include itching, sneezing, and the production of watery rhinorrhea. Oral antihistamine compounds which are selective H1 receptor antagonists, such as chlorphenyramine, cetirizine, desloratidine and fexofenadine are effective in treating the itching, sneezing and rhinorrhea associated with allergic rhinitis. Intranasal antihistamines which are selective H1 receptor antagonists, such azelastine and levocabastine, are thought to have similar therapeutic effects to their oral counterparts. However, such compounds generally require twice daily administration and may still cause sedatation despite their local application.

A class of compounds have been identified as H1 receptor antagonists.

Thus the present invention provides a compound of formula (I)

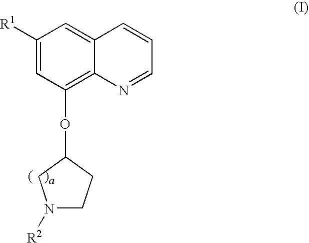

wherein
R¹ represents straight chain C₁₋₆alkyl;
a represents 1 or 2;
R² represents a group of formula (I), (ii), (iii), (iv) or (v) below

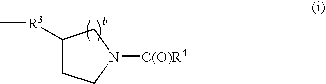

in which R³ represents methylene or ethylene;
b represents 1 or 2; and
R⁴ represents a group selected from —C₁₋₆alkyl (optionally substituted by up to three substituents independently selected from halogen or hydroxy); —C₁₋₆alkylene-O—C₁₋₆alkyl (optionally substituted by up to three substituents independently selected from by halogen and hydroxy); aryl (optionally substituted by up to three substituents independently selected from halogen, C₁₋₃alkyl, trifluoromethyl, and cyano); or —C₁₋₆alkylenearyl (in which the C₁₋₆alkylene is a straight chain and is optionally substituted by up to three substituents independently selected from C₁₋₃alkyl, halogen and hydroxy, and the aryl is optionally substituted by up to three substituents independently selected from halogen, C₁₋₃alkyl, trifluoromethyl, and cyano);

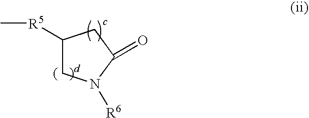

in which R⁵ represents methylene or ethylene;
c represents 0 or 1 and d represents 2 or 3, or c represents 2 or 3 and d represents 0 or 1; and
R⁶ represents hydrogen or C₁₋₆alkyl;

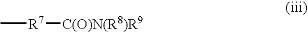

in which R⁷ represents a straight chain C₁₋₆alkylene (optionally substituted by one or two C₁₋₃alkyl), or —CH₂—C₅₋₆cycloalkyl-;
R⁸ represents hydrogen or C₁₋₆alkyl;
R⁹ represents a group selected from hydrogen; —C₁₋₆alkyl (optionally substituted by up to three substituents independently selected from halogen and hydroxy); —C₁₋₆alkylene-O—C₁₋₆alkyl (optionally substituted by up to three substituents independently selected from halogen and hydroxy); C₃₋₇cycloalkyl (optionally substituted by up to three substituents independently selected selected from halogen, hydroxy and C₁₋₃alkyl); —C₁₋₃alkyleneC₃₋₇cycloalkyl (in which the C₁₋₆alkylene is straight chain and optionally substituted by up to three substituents independently selected from C₁₋₃alkyl, halogen and hydroxy, and the C₃₋₇cycloalkyl is optionally substituted by up to three substituents independently selected from halogen, hydroxy and C₁₋₃alkyl); aryl (optionally substituted by up to three substituents independently selected from halogen, C₁₋₃alkyl, trifluoromethyl, and cyano); or —C₁₋₆alkylenearyl (in which the C₁₋₆alkylene is straight chain and is optionally substituted by up to three substituents independently selected from C₁₋₃alkyl, halogen or hydroxy, and the aryl is optionally substituted by up to three substituents independently selected from halogen, C₁₋₃alkyl, trifluoromethyl, and cyano);
or R⁸ and R⁹ together with the N atom to which they are attached represent a 5 to 7 membered saturated heterocyclic ring optionally containing one or two further heteroatoms independently selected from O and S;

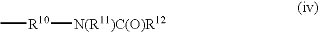

in which R¹⁰ represents a straight chain C₁₋₆alkylene (optionally substituted by one or two C₁₋₃alkyl), or —CH₂—C₅₋₆cycloalkyl;
R¹¹ represents hydrogen or C₁₋₆alkyl; and
R¹² represents a group selected from C₁₋₆alkyl (optionally substituted up to three substituents independently selected from halogen and hydroxy); —C₁₋₆alkylene-O—C₁₋₆alkyl (optionally substituted up to three substituents independently selected from halogen and hydroxy); C₃₋₇cycloalkyl (optionally substituted by up to three substituents independently selected from halogen, hydroxy and C₁₋₃alkyl); —C₁₋₃alkyleneC₃₋₇cycloalkyl (in which the C₁₋₆alkylene is straight chain and optionally substituted by up to three substituents independently selected from C₁₋₃alkyl, halogen and hydroxy, and the C₃₋₇cycloalkyl is optionally substituted by up to three substituents independently selected from halogen, hydroxy and C₁₋₃alkyl); aryl (optionally substituted by up to three substituents independently selected from halogen, C₁₋₃alkyl, trifluoromethyl, and cyano); —C₁₋₆alkylenearyl (in which the C₁₋₆alkylene is straight chain and is optionally substituted by up to three substituents independently selected from C₁₋₃alkyl, halogen or hydroxy, and the aryl is optionally substituted by up to three substituents independently selected from halogen, C₁₋₃alkyl, trifluoromethyl, and cyano);

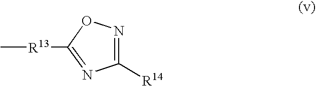

in which R¹³ represents a straight chain C₁₋₆alkylene (optionally substituted by one or two C₁₋₃alkyl), or —CH₂—C₅₋₆cycloalkyl; and

R¹⁴ represents a group selected from hydrogen; —C₁₋₆alkyl (optionally substituted by up to three substituents independently selected from halogen or hydroxy); —C₁₋₆alkylene-O—C₁₋₆alkyl (optionally substituted by up to three substituents independently selected from by halogen and hydroxy); aryl (optionally substituted by up to three substituents independently selected from halogen, C₁₋₃alkyl, trifluoromethyl, and cyano); or —C₁₋₆alkylenearyl (in which the C₁₋₆alkylene is a straight chain and is optionally substituted by up to three substituents independently selected from C₁₋₃alkyl, halogen and hydroxy, and the aryl is optionally substituted by up to three substituents independently selected from halogen, C₁₋₃alkyl, trifluoromethyl, and cyano);

or a salt thereof.

The compounds of the invention may be expected to be useful in the treatment of various diseases in particular inflammatory and/or allergic diseases, such as inflammatory and/or allergic diseases of the respiratory tract (for example allergic rhinitis) that are associated with the release of histamine from cells such as mast cells. Further, the compounds may show an improved profile in that they may possess one or more of the following properties:

(i) greater selectivity over the H3 receptor;
(ii) lower CNS penetration; and
(iii) prolonged duration of action.

Compounds having such a profile may be particularly suitable for intranasal delivery, and/or capable of once daily administration and/or further may have an improved side effect profile compared with other existing therapies.

By ‘selectivity’ it is meant that the compounds may be more potent at the H1 receptor than at the H3 receptor and/or the hERG receptor. The activity at the H1 receptor may be at least about 10 fold greater (e.g. about 100 fold greater) than activity at the H3 receptor.

In another embodiment, R¹ represents straight chain C₄₋₆alkyl;

a represents 1 or 2;
R² represents a group of formula (I), (ii), (iii), (iv) or (v) in which
R³ represents methylene or ethylene;
b represents 2;
R⁴ represents a group selected from —C₁₋₆alkyl; —C₁₋₆alkylene-O—C₁₋₃alkyl; C₄₋₇cycloalkyl; aryl (e.g. phenyl) (optionally substituted by one or two (e.g. one) substituents independently selected from halogen, C₁₋₃alkyl (e.g. methyl), trifluoromethyl, and cyano); or —C₁₋₃alkylenearyl, (e.g. C₁₋₃alkylenephenyl) (in which the C₁₋₃alkylene is a straight chain, and the aryl is optionally substituted by one or two (e.g. one) substituents independently selected from halogen, C₁₋₃alkyl (e.g. methyl), trifluoromethyl, and cyano);
R⁵ represents methylene;
c represents 0 and d represents 3;
R⁶ represents hydrogen or C₁₋₃alkyl (e.g. methyl);
R⁷ represents a straight chain C₁₋₆alkylene (optionally substituted by one methyl), or —CH₂-cyclohexyl-;
R⁸ represents hydrogen or C₁₋₃alkyl (e.g. methyl);
R⁹ represents a group selected from —C₁₋₆alkyl; —C₁₋₆alkylene-O—C₁₋₃alkyl; C₄₋₇cycloalkyl; aryl (e.g. phenyl) (optionally substituted by one or two (e.g. one) substituents independently selected from halogen, C₁₋₃alkyl (e.g. methyl), trifluoromethyl, and cyano); or —C₁₋₃alkylenearyl, (e.g. C₁₋₃alkylenephenyl) (in which the C₁₋₃alkylene is a straight chain, and the aryl is optionally substituted by one or two (e.g. one) substituents independently selected from halogen, C₁₋₃alkyl (e.g. methyl), trifluoromethyl, and cyano);
or R⁸ and R⁹ together represent a 5 or 6 membered saturated heterocyclic ring optionally containing one further heteroatom selected from O and S;
R¹⁰ represents a straight chain C₁₋₆alkylene (optionally substituted by one methyl group);
R¹¹ represents hydrogen or C₁₋₃alkyl (e.g. methyl);
R¹² represents a group selected from —C₁₋₆alkyl; —C₁₋₆alkylene-O—C₁₋₃alkyl; C₄₋₇cycloalkyl; aryl (e.g. phenyl) (optionally substituted by one or two (e.g. one) substituents independently selected from halogen, C₁₋₃alkyl (e.g. methyl), trifluoromethyl, and cyano); or —C₁₋₃alkylenearyl, (e.g. C₁₋₃alkylenephenyl) (in which the C₁₋₃alkylene is a straight chain, and the aryl is optionally substituted by one or two (e.g. one) substituents independently selected from halogen, C₁₋₃alkyl (e.g. methyl), trifluoromethyl, and cyano);
R¹³ represents a straight chain C₁₋₆alkylene (optionally substituted by one methyl group);
R¹⁴ represents a group selected from —C₁₋₆alkyl; —C₁₋₆alkylene-O—C₁₋₃alkyl; C₄₋₇cycloalkyl; aryl (e.g. phenyl) (optionally substituted by one or two (e.g. one) substituents independently selected from halogen, C₁₋₃alkyl (e.g. methyl), trifluoromethyl, and cyano); or —C₁₋₃alkylenearyl, (e.g. C₁₋₃alkylenephenyl) (in which the C₁₋₃alkylene is a straight chain, and the aryl is optionally substituted by one or two (e.g. one) substituents independently selected from halogen, C₁₋₃alkyl (e.g. methyl), trifluoromethyl, and cyano).

In another embodiment, R¹ represents straight chain C₄₋₆alkyl, such as n-butyl, n-pentyl or n-hexyl, e.g. n-hexyl.

In another embodiment, a represents 1. In yet another embodiment, when a represents 1, the stereochemical configuration at the carbon linking the pyrrolidine is R.

In another embodiment, a represents 2.

In another embodiment, R² represents a group of formula (I).

In another embodiment, R² represents a group of formula (ii).

In another embodiment, R² represents a group of formula (iii).

In another embodiment, R² represents a group of formula (iv).

In another embodiment, R² represents a group of formula (v).

In another embodiment, R² represents a group of formula (I) in which R³ represents methylene or ethylene;

b represents 2; and
R⁴ represents a group selected from —C₁₋₆alkyl; —C₁₋₆alkylene-O—C₁₋₃alkyl; C₄₋₇cycloalkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano).

In another embodiment, R² represents a group of formula (II) in which R⁵ represents methylene;

c represents 0 and d represents 3; and
R⁶ represents hydrogen or C₁₋₃alkyl (e.g. methyl).

In another embodiment, R² represents a group of formula (iii) in which R⁷ represents a straight chain C₁₋₄alkylene (optionally substituted by one methyl group) or —CH₂-cyclohexyl;

R⁸ represents hydrogen or C₁₋₃alkyl (e.g. methyl);
R⁹ represents a group selected from —C₁₋₆alkyl; —C₁₋₆alkylene-O—C₁₋₃alkyl; C₄₋₇cycloalkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano);
or R⁸ and R⁹ together represent a 5 or 6 membered saturated heterocyclic ring optionally containing one further heteroatom selected from O and S.

In another embodiment, R² represents a group of formula (iv) in which R¹⁰ represents a straight chain C₁₋₄alkylene (optionally substituted by one methyl group);

R¹¹ represents hydrogen or C₁₋₃alkyl (e.g. methyl); and
R¹² represents a group selected from C₁₋₆alkyl; —C₁₋₆alkylene-O—C₁₋₃alkyl; C₄₋₇cycloalkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano).

In another embodiment R² represents a group of formula (v) in which R¹³ represents a straight chain C₁₋₄alkylene (optionally substituted by one methyl group);

R¹⁴ represents a group selected from —C₁₋₆alkyl; —C₁₋₆alkylene-O—C₁₋₃alkyl; C₄₋₇cycloalkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano).

In another embodiment, R⁸ and R⁹ together represent morpholine, pyrrolidine or piperidine.

In another embodiment, R⁴ represents a group selected from —C₁₋₆alkyl.

In another embodiment, R⁸ represents hydrogen or C₁₋₃alkyl (e.g. methyl);

R⁹ represents a group selected from —C₁₋₆alkyl; —C₁₋₃alkylene-O—C₁₋₃alkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano); or

R⁸ and R⁹ together represent a 5 or 6 membered saturated heterocyclic ring optionally containing one further heteroatom selected from O and S.

In another embodiment, R¹² represents a group selected from —C₁₋₆alkyl; —C₁₋₃alkylene-O—C₁₋₃alkyl; or C₄₋₇cycloalkyl.

In another embodiment, R¹⁴ represents a group selected from —C₁₋₆alkyl.

Representative compounds of formula (I) include the compounds of Examples 1 to 91, including individual isomers thereof and isomeric mixtures (e.g. a racemate or a racemic mixture), in the form of a free base, or as salts thereof (e.g. pharmaceutically acceptable salts thereof).

It is to be understood that the invention includes all possible combinations of embodiments, groups and substituents described herein.

C₁₋₆alkyl, whether alone or as part of another group, may be straight chain or branched and C₁₋₆alkoxy shall be interpreted similarly. Representative examples include, but are not limited to methyl, ethyl, n-propyl, iso-propyl, n-butyl, sec-butyl, iso-butyl, t-butyl, n-pentyl, neo-pentyl and n-hexyl. Particular alkyl and alkoxy groups are C₁₋₃alkyl and C₁₋₃alkoxy. When substituted, C₁₋₆alkyl (e.g. C₁₋₃alkyl) may have up to three substituents, for example, one or two substituents, e.g. one substituent. Representative substituents on alkyl include, but are not limited to, methyl, ethyl, chloro, and/or fluoro.

Representative examples of C₁₋₆alkylene include methlyene [—(CH₂)—], ethylene [—(CH₂)₂—], propylene, [—(CH₂)₃—], butylene [—(CH₂)₄—], pentylene [—(CH₂)₅—] and hexylene [—(CH₂)₆—]. When substituted, C₁₋₆alkylene may have one or two, e.g. one substituent. Representative substituents on C₁₋₆alkylene include, but are not limited to methyl and/or ethyl, e.g. methyl.

As defined herein, the term “aryl” includes single and fused aromatic rings. Representative examples of aryl groups include, but are not limited to phenyl and naphthyl. Aryl is intended to denote all positional isomers thereof. A particular aryl group is phenyl. When substituted, aryl may have up to three substituents, for example, one or two substituents, e.g. one substituent. Representative substituents on aryl include, but are not limited to, methyl, ethyl, chloro, fluoro, trifluoromethyl and/or cyano.

As defined herein, the term “C₃₋₇cycloalkyl” refers to a non-aromatic hydrocarbon ring having from three to seven carbon atoms. “C₅₋₆cycloalkyl” shall be interpreted similarly. Representative examples of such rings include cyclopropyl, cyclobutyl, cyclopentyl, cyclohexyl and cycloheptyl.

The term “halogen” is used herein to describe, unless otherwise stated, a group selected from fluorine, chlorine, bromine or iodine, particularly chlorine or fluorine.

Representative 5 to 7 membered saturated heterocyclic rings containing a nitrogen atom optionally containing one or more (e.g. one) further heteroatoms selected from O and S include, but are not limited to, pyrrolidine, piperidine, homopiperidine, morpholine and thiomorpholine. Particular rings include pyrrolidine, piperidine and morpholine.

It is to be understood that the present invention covers compounds of formula (I) as the free base and as salts thereof, for example as a pharmaceutically acceptable salt.

It is to be further understood that references hereinafter to compounds of the invention or to compounds of formula (I) mean a compound of formula (I) as the free base, or as a salt, unless otherwise stated.

The compounds of formula (I) may be in the form of and/or may be administered as a pharmaceutically acceptable salt. For a review on suitable salts see Berge et al., J. Pharm. Sci., 1977, 66, 1-19. Suitable pharmaceutically acceptable salts include acid addition salts. As used herein, the term “pharmaceutically acceptable salt”, means any pharmaceutically acceptable salt or solvate of a compound of formula (I), which upon administration to the recipient is capable of providing (directly or indirectly) a compound of formula (I), or an active metabolite or residue thereof.

Typically, a pharmaceutically acceptable salt may be readily prepared by using a desired acid as appropriate. The salt may precipitate from solution and be collected by filtration or may be recovered by evaporation of the solvent.

A pharmaceutically acceptable acid addition salt can be formed by reaction of a compound of formula (I) with a suitable inorganic or organic acid (such as hydrobromic, hydrochloric, sulphuric, nitric, phosphoric, succinc, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic (e.g. 2-naphthalenesulfonic), or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

Other non-pharmaceutically acceptable salts, e.g. oxalates or trifluoroacetates, may be used, for example in the isolation of the compounds of formula (I), and are included within the scope of this invention.

The invention includes within its scope all possible stoichiometric and non-stoichiometric forms of the salts of the compounds of formula (I).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or capable of forming hydrogen bonds such as water, xylene, N-methylpyrrolidinone, methanol and ethanol may be used to form solvates. Methods for identification of solvates include, but are not limited to, NMR and microanalysis. Solvates of the compounds of formula (I) are within the scope of the invention.

Compounds of formula (I) may exist in different physical forms. Such forms are within the scope of the present invention. Thus, the compounds of formula (I) may be in a crystalline or amorphous state. Furthermore, if crystalline, the compounds of formula (I) may exist in one or more polymorphic forms, which are included in the scope of the present invention. The most thermodynamically stable polymorphic form, at room temperature, of compounds of formula (I) is of particular interest.

Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (ssNMR).

It will be appreciated that the compounds of formula (I) may possess one or more asymmetric carbon atoms so that optical isomers e.g. enantiomers or diastereoisomers may be formed. The present invention encompasses optical isomers of the compounds of formula (I) whether as individual isomers isolated such as to be substantially free of the other isomer (i.e. pure) or as mixtures thereof (e.g. racemates and racemic mixtures). An individual isomer isolated such as to be substantially free of the other isomer (i.e. pure) may be isolated such that less than about 10%, particularly less than about 1%, for example less than about 0.1% of the other isomer is present.

Further, it will be appreciated that the R and S enantiomers may be isolated from the racemate by conventional resolution methods such as preparative HPLC involving a chiral stationary phase, by resolution using fractional crystallisation of a salt of the free base with a chiral acid, by chemical conversion to a diastereoisomer using a chiral auxiliary followed by chromatographic separation of the isomers and then removal of the chiral auxiliary and regeneration of the pure enantiomer, or by total asymmetric synthesis.

Further it will be appreciated that the compounds of formula (I) may form geometric isomers, including cis and trans configurations. The present invention includes such geometric isomers, whether as individual isomers isolated such as to be substantially free of the other isomers (i.e. pure) or as mixtures thereof. Thus for example the present invention encompases an individual geometric isomer isolated such as to be substantially free of the other isomer (i.e. pure) such that less than 10%, for example less than 1% or less than 0.1% of the other isomer is present. Separation of geometric isomers may be achieved by conventional techniques, e.g. by fractional crystallisation, chromatography or HPLC. When R⁷ represents —CH₂—C₅₋₆cycloalkyl-, a particular geometric isomer of the invention is the trans isomer.

Certain compounds of formula (I) may exist in one of several tautomeric forms. It will be understood that the present invention encompasses tautomers of the compounds of formula (I) whether as individual tautomers or as mixtures thereof.

It will be appreciated from the foregoing that included within the scope of the invention are solvates (e.g. hydrates), isomers (geometric and optical) and polymorphic forms of the compounds of formula (I) and salts thereof.

There is also provided processes for the preparation of compounds of formula (I) or salts thereof.

For the avoidance of doubt, throughout the process section, unless otherwise stated, (CH₂)ₙ corresponds to the C₁₋₆alkylene defined in R² in the compound of formula (I), and thus may be optionally substituted by one C₁₋₃alkyl group.

There is also provided processes for the preparation of compounds of formula (I) or salts thereof.

According to a first process, A, a compound of formula (I) in which R² represents a group of formula (i) may be prepared by reacting a compound of formula (II)

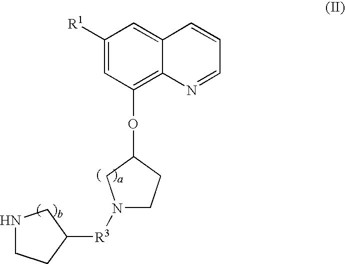

with a compound of formula (III)

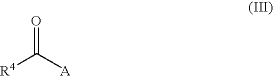

wherein R¹, a, b, R³ and R⁴ are as defined hereinabove for formula (I), and A represents chlorine or hydroxy.

When A represents chlorine, the reaction may typically be carried out in a suitable solvent, such as dichloromethane (DCM), with an appropriate base, e.g. triethylamine.

When A represents hydroxy, the reaction may typically be carried out in a suitable solvent such as N,N′-dimethylformamide (DMF), with an appropriate base, e.g. triethylamine or N,N′ diisopropylethylamine (DIPEA) and in the presence of a suitable activating agent such as O-(benzotriazol-1-yl)-N,N,N′,N′-tetramethyluronium tetrafluoroborate (TBTU), benzotriazol-1-yl-oxytripyrrolidinophosphonium hexafluorophosphate (PyBop) or 2-(1H-7-azabenzotriazol-1-yl)-1,1,3,3-tetramethyl uronium hexafluorophosphate (HATU). The reaction is optionally heated, for example using a microwave oven at an appropriate elevated temperature, for example from about 140 to 160° C. for approximately 10 to 40 min, as appropriate.

Compounds of formula (II) may be prepared according to Scheme 1 below.

Compounds of formula (III) in which X represents chlorine are commercially available, for example from Aldrich and/or Alfa Aesar and/or Apollo, and include acetyl chloride, 3,3,3-trifluoropropionyl chloride, hexanoyl chloride, 2-bromopropionyl chloride, methoxyacetyl chloride, benzoyl chloride, 3-fluorobenzoyl chloride, m-toluoyl chloride, 4-(trifluoromethyl)benzoyl chloride, 4-cyanobenzoyl chloride, 3,5-bis(trifluoromethyl)benzoyl chloride, 3-cyano-4-fluorobenzoyl chloride, 4-phenylbutyryl chloride and 2-(4-chlorophenyl)-3-methylbutyryl chloride.

Compounds of formula (III) in which X represents hydroxy are commercially available, for example from Aldrich and/or TCI-Europe and/or Acros and/or Chembridge and/or Milestone Pharmtech, and include formic acid, 3-hydroxy-2,2-dimethylpropanoic acid, methoxyacetic acid, 3-methoxypropionic acid, 4-(2,4-dimethylphenyl)-4-hydroxybutanoic acid, 4-(4-fluorophenyl)-4-hydroxybutanoic acid, 3-(trifluoromethyl)butyric acid, 4,4,4-trifluorobutyric acid, 7-phenylheptanoic acid, and 3-(4-cyanophenyl)propanoic acid.

Compounds of formula (III) in which X represents hydroxy may also be prepared by methods well-known to those skilled in the art, for example, by hydrolysis of a corresponding ester. The reaction may typically be carried out using an appropriate base e.g. sodium hydroxide in a suitable solvent such as methanol or ethanol. Examples of commercially available corresponding esters include ethyl 3-ethoxypropionate, ethyl 4-ethoxybutyrate and methyl 4-(methyloxy)butanoate, which are commercially available, for example, from Aldrich.

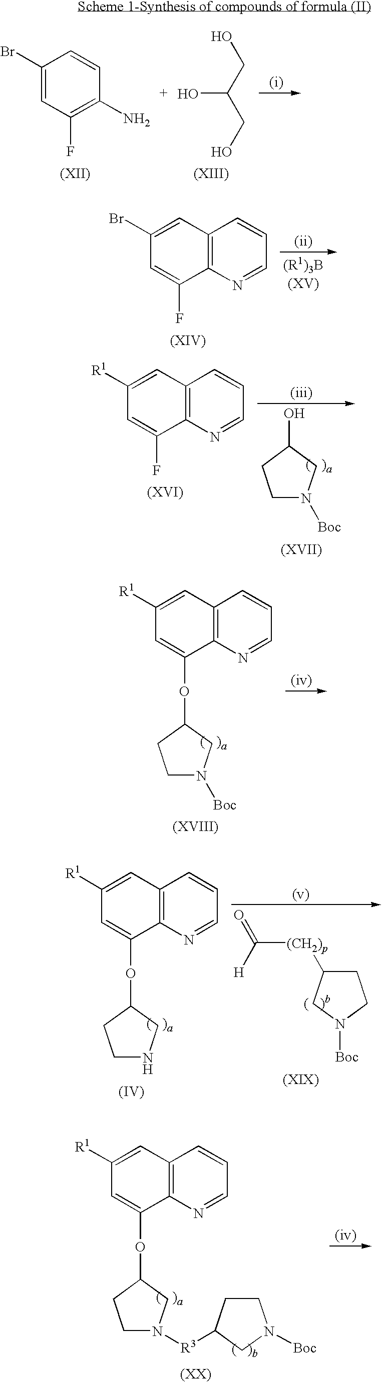

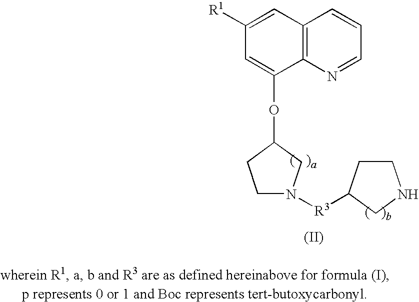

Reagents and Conditions: i) suitable acid e.g. concentrated sulphuric acid, appropriate solvent such as water, sodium 3-nitrobenzenesulfonate (commercially available, for example, from Aldrich), appropriate elevated temperature such as from about 110 to 140° C.; ii) suzuki reaction using a suitable solvent such as DMF and/or tetrahydrofuran (THF), suitable base e.g. potassium carbonate, appropriate catalyst for example [1,1′-bis(diphenylphosphino) ferrocene palladium (II)]chloride, at an elevated temperature such as from about 70 to 80° C.; iii) suitable solvent such as N-methylpyrrolidinone (NMP), appropriate base e.g. sodium tert-butoxide, at an elevated temperature for example from about 130 to 150° C.; iv) deprotection using a suitable acid e.g. trifluoracetic acid (TFA) or hydrogen chloride (HCl) in a suitable solvent such as DCM or 1,4-dioxane at ambient temperature; v) Reductive amination using a suitable solvent such as DCM, an appropriate reducing agent such as sodium triacetoxyborohydride, suitable catalyst e.g. acetic acid.

Alternatively, step ii) in Scheme 1 may be carried out using 9-borabicyclo[3.3.1]nonane and an appropriate olefin to make a boron compound (equivalent to compound (XV)) in situ. The reaction is typically carried out in a suitable solvent such as THF with an appropriate catalyst e.g. 1,1′-bis(diphenylphosphino)ferrocene]dichloropalladium(II). The reaction is carried out in a manner similar to that described by S. Potuzak and D. S. Tan, Tetrahedron Lett., 45:1797-1801, (2004). Olefins are commercially available, for example, from Aldrich and include ethene, 1-propene, 1-butene, 1-pentene and 1-hexene.

The compound of formula (XII), 4-bromo-2-fluoroaniline is commercially available, for example, from Aldrich.

The compound of formula (XIII), glycerol, is commercially available, for example, from Fluka and/or Aldrich.

The compounds of formula (XV) are commercially available, for example from Aldrich, and include trimethylboron, triethylborane and tributylborane.

Compounds of formula (XVII) are commercially available, for example from Aldrich and include N-tert-butoxycarbonyl-(R)-(−)-3-pyrrolidinol, N-tert-butoxycarbonyl-(S)-(+)-3-pyrrolidinol and tert-butyl 4-hydroxy-1-piperidinecarboxylate.

Compounds of formula (XVIII) may also be prepared according to Scheme 2 below.

Compounds of formula (IV) may also be prepared according to Scheme 3 below.

Compounds of formula (XIX) are commercially available, for example from ABCR and/or Betapharma, and include 1-Boc-3-pyrrolidinecarbaldehyde, 4-formyl-piperidine-1-carboxylic acid tert-butyl ester and 4-(2-oxo-ethyl)-piperidine-1-carboxylic acid tert-butyl ester.

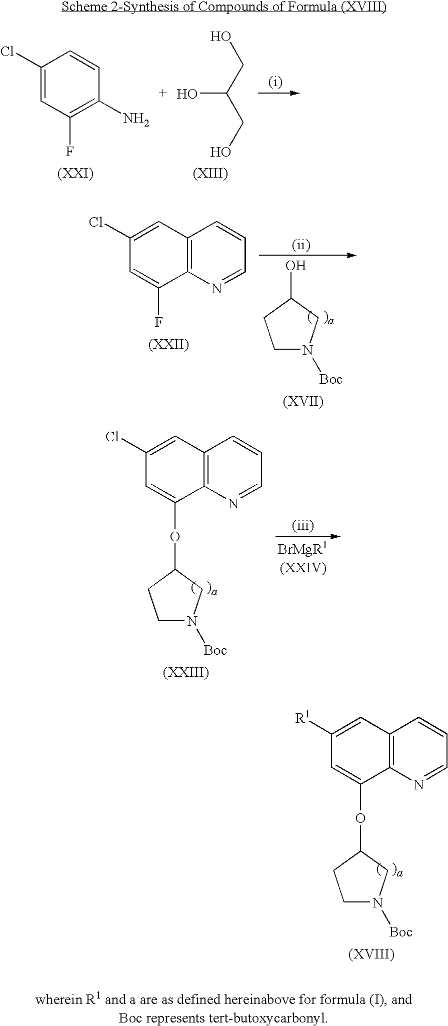

Reagents and Conditions: i) suitable acid e.g. concentrated sulphuric acid, appropriate solvent such as water, sodium 3-nitrobenzenesulfonate (commercially available, for example, from Aldrich), appropriate elevated temperature such as from about 110 to 140° C.; ii) suitable solvent such as NMP, appropriate base e.g. sodium tert-butoxide, at an appropriate elevated temperature for example from about 130 to 150° C.; iii) suitable solvent such as THF:NMP (10:1) at an appropriate lowered temperature e.g. from about 0 to 5° C., using a suitable catalyst for example iron(III) acetylacetonate, preferably in an inert, water-free atmosphere.

Alternatively, Step (i) of Scheme 2 may be carried out using acrolein (commercially available, for example, from Aldrich) instead of the compound of formula (XIII) (glycerol). The reaction may be carried out in a suitable solvent, such as 1-butanol, with an appropriate acid e.g. hydrochloric acid. To aid the reaction, 4-chloroaniline (commercially available, for example, from Aldrich) may be added and the reaction may be heated to an appropriate elevated temperature, for example, from about 110 to 140° C., for a suitable length of time, for example for about 5 minutes, as appropriate.

The compound of formula (XXI), 4-chloro-2-fluoroaniline, is commercially available, for example, from Aldrich.

Compounds of formula (XIII) and (XVII) are commercially available, see above (after Scheme 1).

Compounds of formula (XXIV) are commercially available, for example, from Aldrich and/or TCI-Europe and include methylmagnesium bromide, ethylmagnesium bromide, n-propylmagnesium bromide, n-butylmagnesium bromide, n-pentylmagnesium bromide and n-hexylmagnesium bromide.

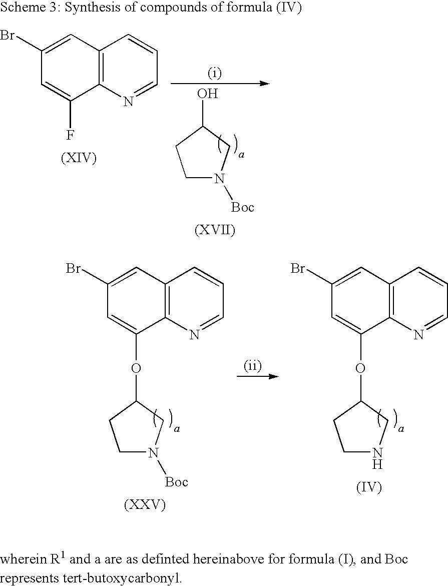

Reagents and Conditions: i) suitable solvent such as NMP, appropriate base e.g. sodium tert-butoxide, at an appropriate elevated temperature for example from about 130 to 150° C.; ii) pre-reaction of 9-borabicyclo[3.3.1]nonane in a suitable solvent such as THF with an appropriate olefin, to make a boron compound (equivalent to compound (XIII)) in situ. Followed by addition of a suitable base to neutralise e.g. 1 equivalent of aqueous sodium hydroxide. Followed by addition of compound of formula (XXIV) and an appropriate catalyst e.g. 1,1′-bis(diphenylphosphino)ferrocene]dichloropalladium(II). The reaction is similar to that described by S. Potuzak and D. S. Tan, Tetrahedron Lett., 45:1797-1801, (2004). Olefins are commercially available, for example, from Aldrich and include ethene, 1-propene, 1-butene, 1-pentene and 1-hexene.

Compounds of formula (XIV) may be prepared according to Scheme 1, above.

Compounds of formula (XVII) are commercially available, see above (after Scheme 1).

According to a second process, B, a compound of formula (I) in which R² represents a group of formula (ii) may be prepared by reacting a compound of formula (IV)

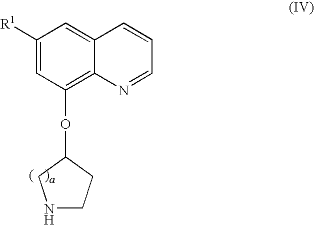

with a compound of formula (V)

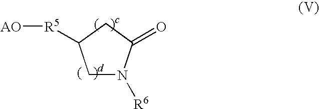

wherein R¹, a, c, d, R⁵ and R⁶ are as defined hereinabove for formula (I), and OA represents an activated hydroxy group such as a mesylate or tosylate.

The reaction may typically be carried out in a suitable solvent, such as DMF, with an appropriate base, e.g. sodium bicarbonate (sodium hydrogen carbonate) and in the presence of a suitable activating agent such as sodium iodide. The reaction is usually heated, for example using a microwave oven at an appropriate elevated temperature, for example from about 140 to 160° C. for approximately 10 to 40 min, as appropriate.

Compounds of formula (IV) may be prepared according to Scheme 1 and Scheme 3, above.

Compounds of formula (V) may be prepared according to Scheme 4 below.

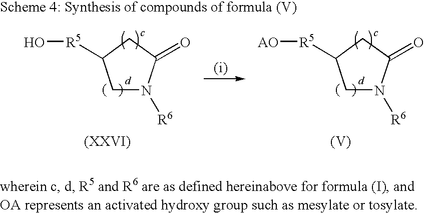

Reagents and Conditions: i) suitable solvent such as DCM, appropriate base e.g. triethylamine, activating agent for example methanesulfonyl chloride or p-toluenesulfonyl chloride (both commercially available, for example, from Aldrich).

Compounds of formula (XXVI) are commercially available and/or may be prepared according to methods well-known to those skilled in the art. Examples of compounds which are commercially available include (R)-(−)-5-(hydroxymethyl)-2-pyrrolidinone and (S)-(+)-5-(hydroxymethyl)-2-pyrrolidinone are available, for example, from Aldrich. 5-(2-hydroxyethyl)-2-pyrrolidinone may be prepared according to methods disclosed in European Patent EP 537606 B1, see Example 2. 4-(hydroxymethyl)-2-pyrrolidinone may be prepared according to methods disclosed in German Patent Application DE 2557335 A1, see Example 1. (S)-4-(2-hydroxyethyl)-2-pyrrolidinone may be prepared according to methods disclosed by Hanessian, S., et al., J. Org. Chem., 58(19):5032-5034, (1993), see Chart 1. 3-(2-hydroxyethyl)-2-pyrrolidinone may be prepared according to methods disclosed by Otto, A., et al., Tetrahedron Asymmetry, 10(17):3381-3389, (1999), see compound 7a. 6-(hydroxymethyl)-piperidin-2-one may be prepared according to methods disclosed by Synthetic Comm., 26(4):687-696, (1996). 6-(2-hydroxyethyl)-2-piperidinone may be prepared according to methods disclosed by Mohammad, T., et al., J. Label. Compds. and Radiopharmaceuticals, 28(9):1087-1092, (1990). 5-(hydroxymethyl)-2-piperidinone may be prepared according to methods disclosed by Lerchner, A., et al., Chemistry—A European Journal, 12(32):8208-8219, (2006), see compound 30. 3-(hydroxymethyl)-2-piperidinone may be prepared according to methods disclosed by Smith, R. D., et al., J. Med. Chem., 24:104, (1981), see compound 2a. 7-hydroxymethyl-azepan-2-one may be prepared according to methods disclosed in International Patent Application WO 2006/103255 A1, see Compound F1. Hexahydro-7-(2-hydroxyethyl)-2H-azepin-2-one may be prepared according to methods disclosed in Can. J. Chem., 49(10):1648-1658, (1971). Hexahydro-3-(2-hydroxyethyl)-2H-azepin-2-one may be prepared according to methods disclosed by Cummings, W. A. W. et al., J. Chem. Soc., 4591-4604, (1964), see compound (VIII).

According to a third process, C, a compound of formula (I) in which R² represents a group of formula (iii) may be prepared by reacting a compound of formula (VI)

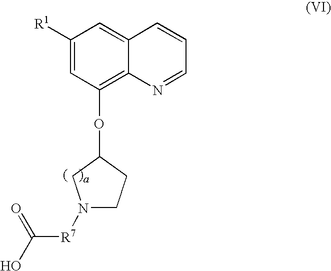

with a compound of formula (VII)

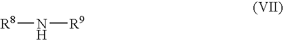

wherein R¹, a, R⁷, R⁸ and R⁹ are as defined hereinabove for formula (I).

The reaction may typically be carried out in a suitable solvent, such as DMF, with an appropriate base, e.g. triethylamine or DIPEA and in the presence of a suitable activating agent such as TBTU, PyBop or HATU. The reaction is usually heated, for example using a microwave oven at an appropriate elevated temperature, for example from about 140 to 160° C. for approximately 10 to 40 min, as appropriate.

Compounds of formula (VI) may be prepared according to Scheme 5, Scheme 6 or Scheme 7, below.

Compounds of formula (VII) are commercially available, for example, from Aldrich and/or ABCR and/or Enamine and/or Chembridge and/or GL Synthesis, and include ammonia, methylamine, (R)-(−)-2-aminobutane, hexylamine, dimethylamine, dihexylamine, 2-fluoroethylamine, 2,2-difluoroethylamine, 2,2,2-trifluoroethylamine, 2-aminoethanol, cyclopropylamine, cycloheptylamine, N-methylcyclohexylamine, aniline, 2-aminobenzonitrile, 2-fluoroaniline, 2-aminobenzotrifluoride, 3-amino-4-fluorobenzotrifluoride, N-hexylaniline, benzylamine, (5-phenylbutyl)methylamine hydrochloride, pyrrolidine, thiazolidine, morpholine, 3-ethoxypropylamine, 1-amino-3-methoxy-propan-2-ol and 6,7-dihydroxy-4-oxa heptylamine.

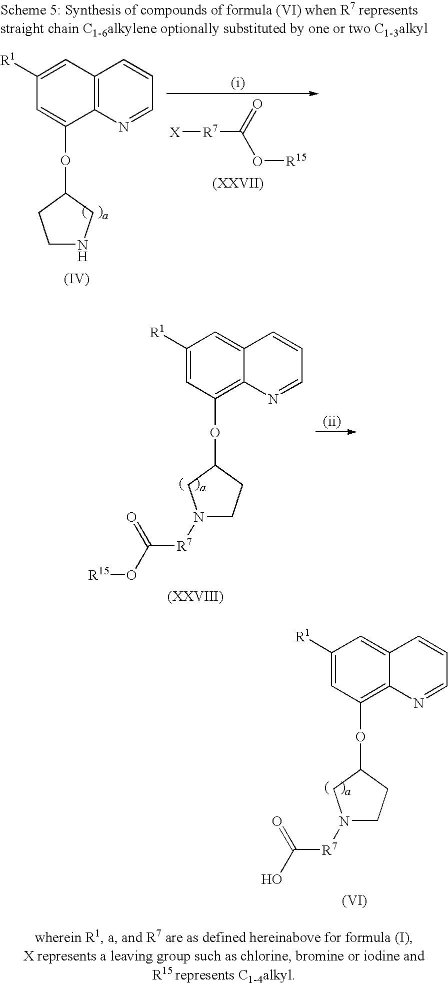

Reagents and Conditions: i) suitable solvent such as DMF or THF, appropriate base e.g. potassium carbonate or DIPEA, appropriate elevated temperature such as from about 50 to 70° C. for an appropriate length of time, such as overnight; ii) ester hydrolysis using an appropriate base such as aqueous sodium hydroxide or lithium hydroxide in a suitable solvent e.g. methanol/water or THF/water.

Compounds of formula (IV) may be prepared according to Scheme 1 and Scheme 3, above.

Compounds of formula (XXVII) are commercially available, for example from Aldrich and/or TCI Europe, and include ethyl 2-bromoacetate, methyl 3-bromopropanoate, ethyl 2-bromopropionate, ethyl 2-bromobutanoate, ethyl 2-bromopentanoate, ethyl 4-bromobutanoate, methyl(R)-(+)-3-bromoisobutyrate, ethyl 5-bromopentanoate, ethyl 6-bromohexanoate and ethyl 7-bromoheptanoate.

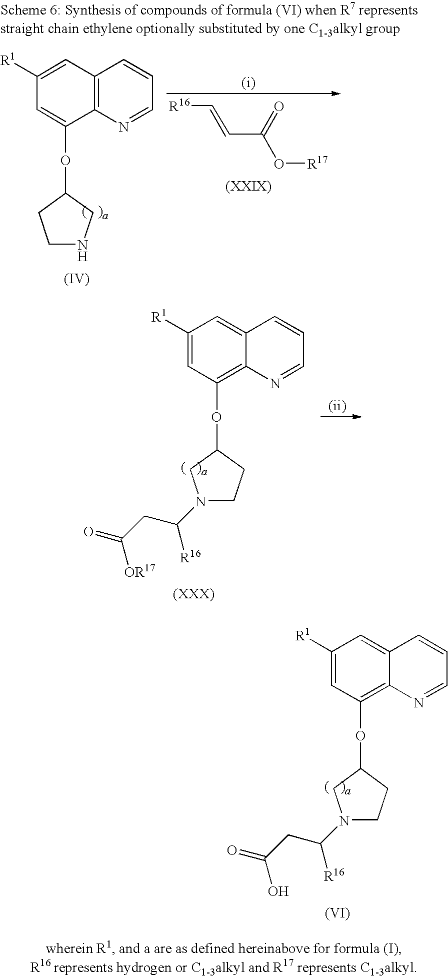

Reagents and Conditions: i) suitable solvent such as THF, appropriate elevated temperature such as from about 50 to 70° C. for an appropriate length of time, such as about 3 to 4 hours; ii) ester hydrolysis using an appropriate base such as aqueous sodium hydroxide or lithium hydroxide in a suitable solvent e.g. methanol/water or THF/water.

Compounds of formula (IV) may be prepared according to Scheme 1 and Scheme 3, above.

Compounds of formula (XXIX) are commercially available, for example from Aldrich and/or Alfa Aesar and/or Rarechem, and include methyl acrylate, ethyl acrylate, ethyl crotonate, ethyl trans-2-pentenoate, ethyl 4-methyl-trans-2-pentenoate and ethyl trans-2-hexenoate.

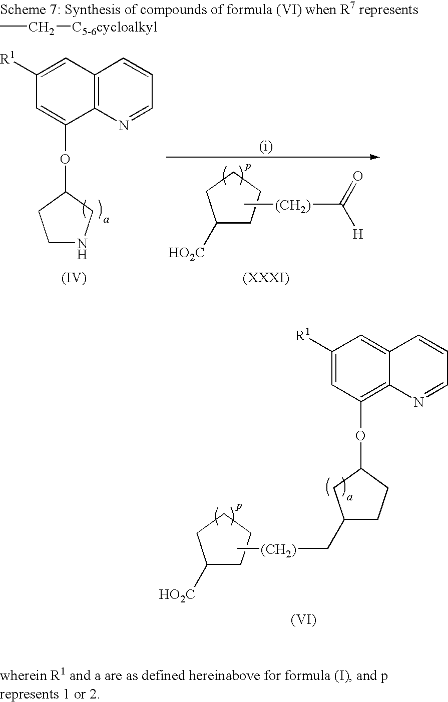

Reagents and Conditions: i) reductive amination using a suitable solvent such as DCM, acid e.g. acetic acid, appropriate reducing agent for example sodium triacetoxyborohydride.

Compounds of formula (IV) may be prepared according to Scheme 1 and Scheme 3, above.

Compounds of formula (XXXI) are commercially available, for example, from Davos, and include 4-formylcyclohexanecarboxylic acid. Other compounds of formula (XXXI) may be prepared according to methods well known to those skilled in the art. For example, 3-formyl-cyclopentanecarboxylic acid may be prepared according to methods disclosed in European Patent EP 0021118 B1, see example 8. trans-2-Formyl-cyclohexanecarboxylic acid may be prepared according to methods disclosed by Moser, W. H. and Hegedus, L. S., J. Am. Chem. Soc., 118(34):7873-7880, (1996), see compound 13.

According to a fourth process, D, a compound of formula (I) in which R² represents a group of formula (iv) may be prepared by reacting a compound of formula (VIII)

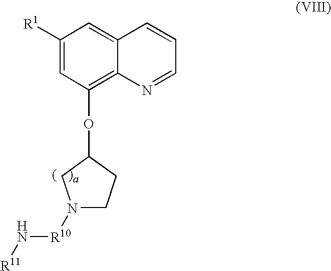

with a compound of formula (IX)

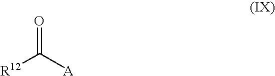

wherein R¹, a, R¹⁰, R¹¹ and R¹² are as defined hereinabove for formula (I), and A represents chlorine or hydroxy.

When A represents chlorine, the reaction may typically be carried out in a suitable solvent, such as DCM, with the addition of a suitable base such as triethylamine.

When A represents hydroxy, the reaction may typically be carried out in a suitable solvent such as DMF, with an appropriate base, e.g. triethylamine or DIPEA and in the presence of a suitable activating agent such as TBTU, PyBop or HATU. The reaction is optionally heated, for example using a microwave oven at an appropriate elevated temperature, for example from about 140 to 160° C. for approximately 10 to 40 min, as appropriate.

Compounds of formula (VIII) may be prepared according to Scheme 8 and Scheme 9, below.

Compounds of formula (IX) in are equivalent to compounds of formula (III), and are commercially available (see Process A).

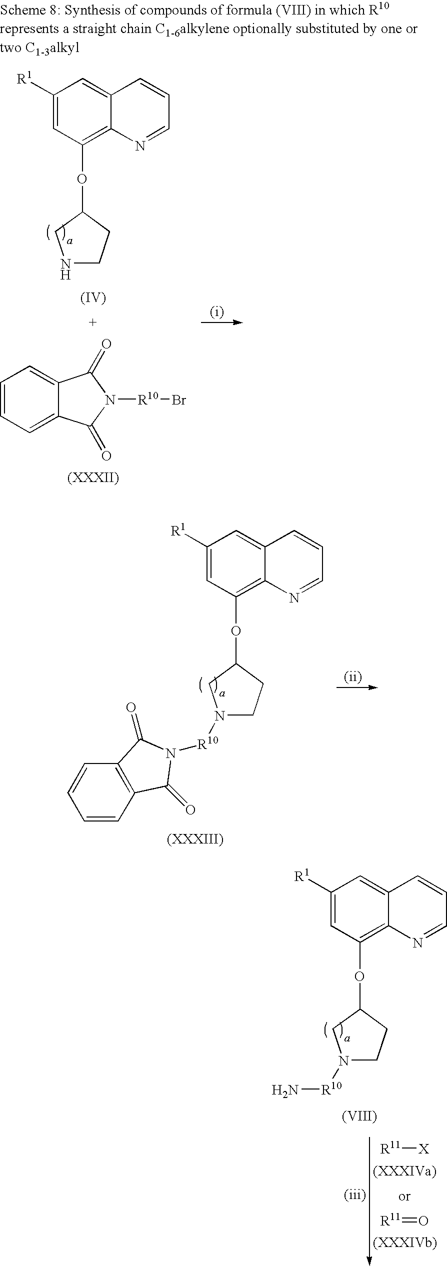

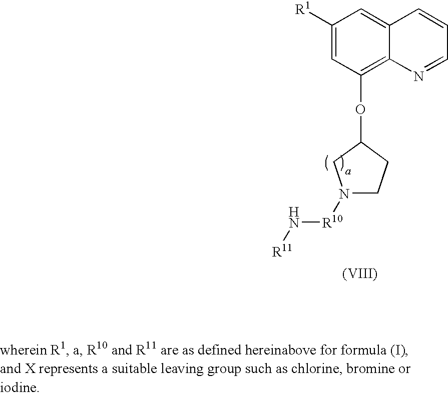

Reagents and Conditions: i) suitable solvent such as 2-butanone, appropriate base e.g. potassium carbonate, at an elevated temperature such as from about 70 to 90° C.; ii) suitable solvent such as ethanol, hydrazine or hydrazine monohydrate, at an elevated temperature such as from about 70 to 90° C.; iii) 1 equivalent of R¹¹—X (XXXIVa), in an appropriate solvent such as DMF, suitable base such as triethylamine or sodium hydride, optionally with an activating agent such as sodium iodide; or reductive amination using R¹¹═O (XXXIVb), in a suitable solvent e.g. DMF, suitable reducing agent such as sodium triacetoxyborohydride.

Compounds of formula (IV) may be prepared according to Scheme 1 and Scheme 3, above.

Compound of formula (XXXII) are commercially available, for example from Acros and/or Aldrich and include 2-(2-bromoethyl)-1H-isoindole-1,3(2H)-dione, N-(bromomethyl) phthalimide, N-(3-bromopropyl)phthalimide, N-(4-bromobutyl)phthalimide, N-(5-bromopentyl)phthalimide and N-(6-bromohexyl)phthalimide.

Compounds of formula (XXXIVa) are commercially available, for example from Aldrich, and include methyl iodide, iodoethane, 1-iodopropane, 1-iodobutane, 1-iodopentane and 1-iodohexane.

Compounds of formula (XXXIVb) are commercially available, for example, from Aldrich, and include formaldehyde, acetaldehyde, propionaldehyde, methyl ethyl ketone, butyraldehyde, valeraldehyde, 3-pentanone, hexanel, 3-hexanone and 3-methyl-3-pentanone.

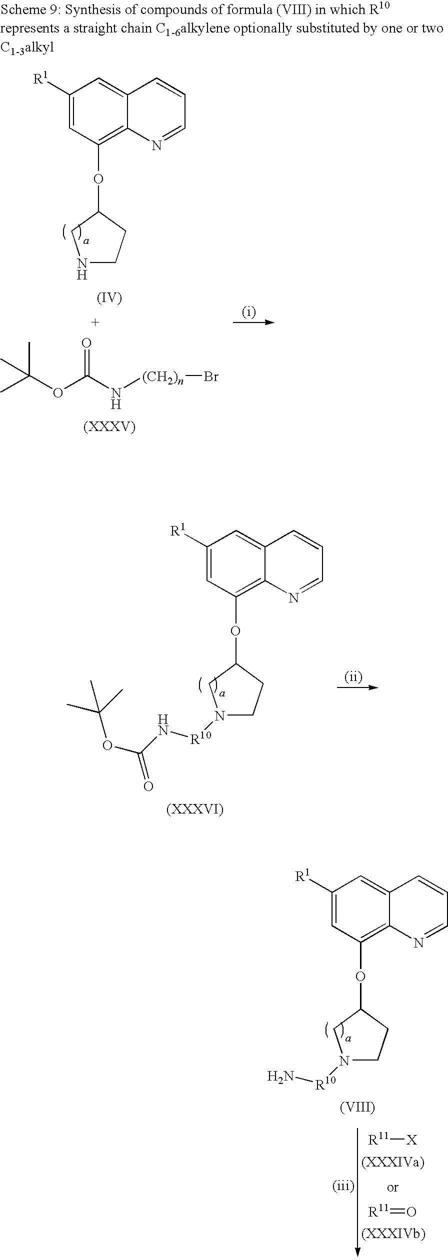

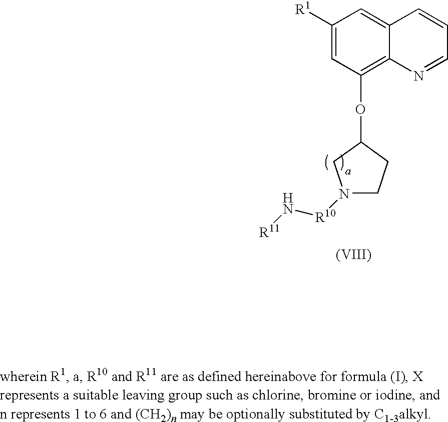

Reagents and Conditions: i) suitable solvent such as 2-butanone, appropriate base e.g. potassium carbonate, at an elevated temperature such as from about 70 to 90° C.; ii) deprotection using a suitable acid such as hydrogen chloride or TFA in a suitable solvent e.g. dioxane or DCM; iii) 1 equivalent of R¹¹—X (XXXIVa), in an appropriate solvent such as DMF, suitable base such as triethylamine or sodium hydride, optionally with an activating agent such as sodium iodide; or reductive amination using R¹¹═O (XXXIVb), in a suitable solvent e.g. DMF, suitable reducing agent such as sodium triacetoxyborohydride.

Compounds of formula (IV) may be prepared according to Scheme 1 and Scheme 3, above.

Compounds of formula (XXXV) are commercially available, for example, from Aldrich and/or Toronto Chemicals, and include 2-(Boc-amino)ethyl bromide, 3-(Boc-amino)propyl bromide, 4-(Boc-amino)butyl bromide, 5-(Boc-amino)pentyl bromide and 6-(Boc-amino)hexyl bromide.

Compounds of formula (XXXIVa) and (XXXIVb) are commercially available, see above (after Scheme 8).

According to a fifth process, E, a compound of formula (I), in which R² represents a group of formula (v) may be prepared by reacting a compound of formula (X)

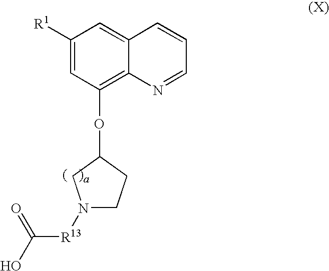

with a compound of formula (XI)

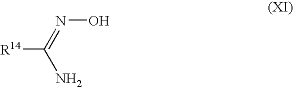

wherein R¹, a, R¹³ and R¹⁴ are as defined hereinabove for formula (I).

The reaction may typically be carried out in a suitable solvent, such as DMF, with an appropriate base, e.g. triethylamine and in the presence of a suitable activating agent such as TBTU. The reaction is usually heated, for example using a microwave oven at an appropriate elevated temperature, for example from about 80 to 100° C. for approximately 5 to 40 min, as appropriate.

Compounds of formula (X) may be prepared according to Schemes 5, 6, and 7 above, in which R⁷ is R¹³.

Compounds of formula (XI) are be commercially available, for example, from Apollo and/or Aldrich and include butyramide oxime, benzamidoxime, 4-(trifluoromethyl)benzamidoxime, 4-methylbenzamide oxime and 4-bromo-N′-hydroxybenzenecarboximidamide. Alternatively, compounds of formula (XXXIII) may be prepared according to Scheme 10, below.

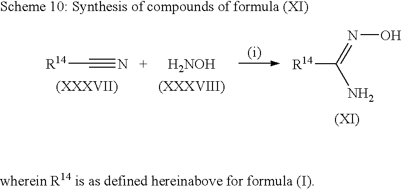

Reagents and Conditions: i) suitable solvent such as ethanol, toluene or THF, optionally with the addition of an appropriate base such as aqueous sodium carbonate or triethylamine.

Compounds of formula (XXXVII) are commercially available, for example, from Aldrich and/or Alfa Aesar and include acetonitrile, hexanenitrile, 3-hydroxypropionitrile, trifluoroacetonitrile, 3-methoxypropionitrile, 3-(2,2,2-trifluoroethoxy)propionitrile, benzonitrile, 2-chlorobenzonitrile, 2-chloro-6-methylbenzonitrile, 4-pentylbenzonitrile, 4-(trifluoromethyl)benzonitrile, isophthalonitrile, 4-phenylbutyronitrile and 3-(3-chloro-phenyl)-propionitrile.

The compound of formula (XXXVIII), hydroxylamine, or hydroxylamine hydrochloride are commercially available, for example, from Aldrich.

According to a sixth process, F, a compound of formula (I) may be prepared by interconversion from other compounds of formula (I).

Interconversions include, but are not limited to alkylation and deprotection, under standard conditions well known to those skilled in the art.

Thus, typically, an alkylation reaction may be carried out between a compound of formula (I) and a C₁₋₆alkyl, activated to substitution by means of a leaving group such as halogen or an activated hydroxy group, such as mesylate or tosylate. The reaction usually takes place in the presence of a suitable base such as triethylamine, N,N′-diisopropylethylamine or sodium hydride, in an appropriate solvent such as 2-butanone or DMF, at an appropriate elevated temperature such as at about 80° C. or at room temperature.

According to a sixth process, F, a salt of a compound of formula (I) may be prepared by exchange of counterions, or precipitation of said salt from the free base.

Examples of protecting groups that may be employed in the synthetic routes described and the means for their removal can be found in T. W. Greene et al., ‘Protective Groups in Organic Synthesis’ (3rd edition, J. Wiley and Sons, 1999). Suitable amine protecting groups include sulfonyl (e.g. tosyl), acyl (e.g. acetyl, 2′,2′,2′-trichloroethoxycarbonyl, benzyloxycarbonyl or tert-butoxycarbonyl) and arylalkyl (e.g. benzyl), which may be removed by hydrolysis (e.g. using an acid such as hydrogen chloride in dioxane or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃), which may be removed by base catalysed hydrolysis or a solid phase resin bound benzyl group, such as a Merrifield resin bound 2,6-dimethoxybenzyl group (Ellman linker), which may be removed by acid cleavage, for example with trifluoroacetic acid.

It will be appreciated that novel intermediates described herein form another embodiment of the present invention.

Compounds of formula (I) or a pharmaceutical acceptable salt thereof may be useful for the treatment of various inflammatory and/or allergic diseases.

Examples of disease states in which a compound of formula (I), or a pharmaceutically acceptable salt thereof may have potentially beneficial anti-inflammatory and/or anti-allergic effects include inflammatory and/or allergic diseases of the respiratory tract, such as allergic rhinitis (seasonal and perennial) or other diseases such as bronchitis (including chronic bronchitis), asthma (including allergen-induced asthmatic reactions), chronic obstructive pulmonary disease (COPD) and sinusitis.

Furthermore, the compounds of formula (I) may be of use in the treatment of nephritis, skin diseases such as psoriasis, eczema, allergic dermatitis and hypersensitivity reactions. Also, the compounds of formula (I) may be useful in the treatment of insect bites and stings.

The compounds of formula (I) may also be of use in the treatment of nasal polyposis, conjunctivitis (e.g. allergic conjunctivitis) or pruritis.

A disease of particular interest is allergic rhinitis.

Other diseases in which histamine may have a pathophysiological role include non-allegic rhinitis, and also diseases of the gastrointestinal tract such as intestinal inflammatory diseases including inflammatory bowel disease (e.g. Crohn's disease or ulcerative colitis) and intestinal inflammatory diseases secondary to radiation exposure or allergen exposure.

It will be appreciated by those skilled in the art that references herein to treatment or therapy may extend to prophylaxis as well as the treatment of established conditions.

As mentioned above, compounds of formula (I) may be useful as therapeutic agents. There is thus provided a compound of formula (I) or a pharmaceutically acceptable salt thereof for use in therapy.

In another embodiment, there is provided a compound of formula (I) or a pharmaceutically acceptable salt thereof for use in the treatment of inflammatory and/or allergic diseases (such as any of the above diseases, in particular allergic rhinitis).

In another embodiment, there is provided the use of a compound of formula (I) or a pharmaceutically acceptable salt thereof for the manufacture of a medicament for the treatment of inflammatory and/or allergic diseases (such as any of the above diseases, in particular allergic rhinitis).

In another embodiment, there is provided a method for the treatment (or prophylaxis) of inflammatory and/or allergic diseases (such as any of the above diseases, in particular allergic rhinitis), which method comprises administering to a patient in need thereof an effective amount of a compound of formula (I) or a pharmaceutically acceptable salt thereof.

When used in therapy, the compounds of formula (I) or pharmaceutically acceptable salts thereof may typically be formulated in a suitable pharmaceutical composition. Such pharmaceutical compositions may be prepared using standard procedures.

Thus, there is provided a composition which comprises a compound of formula (I) or a pharmaceutically acceptable salt thereof and one or more (e.g. 10 or fewer) pharmaceutically acceptable carriers and/or excipients.

A composition comprising a compound of formula (I) or a pharmaceutically acceptable salt thereof, which may be prepared by admixture, suitably at ambient temperature and atmospheric pressure, may be suitable for topical administration (which includes epicutaneous, inhaled, intranasal or ocular administration), enteral administration (which includes oral or rectal administration) or parenteral administration (such as by injection or infusion). Of interest are compositions comprising a compound of formula (I) or a pharmaceutically acceptable salt thereof, suitable for topical administration, particularly suitable for intranasal administration.

Generally, compositions may be in the form of solutions or suspensions (aqueous or non-aqueous), tablets, capsules, oral liquid preparations, powders, granules, lozenges, lotions, creams, ointments, gels, foams, reconstitutable powders or suppositories as required by the route of administration.

Generally, the compositions comprising a compound of formula (I) or a pharmaceutically acceptable salt thereof may contain from about 0.001% to 99% (w/w), such as from about 0.1 to 60% (w/w) (based on the total weight of the composition), of the compound of formula (I) or the pharmaceutically acceptable salt thereof, depending on the route of administration. The dose of the compound used in the treatment of the aforementioned diseases will vary in the usual way with the seriousness of the diseases, the weight of the sufferer, and other similar factors. However, as a general guide, suitable unit doses may be about 0.05 to 1000 mg, for example about 0.05 to 200 mg, and such unit doses may be administered more than once a day, for example two or three times a day or as desired. Such therapy may extend for a number of weeks or months.

The proportion of the compound of formula (I) or a pharmaceutically acceptable salt thereof in a topical composition will depend on the precise type of composition to be prepared and the particular route of administration, but will generally be within the range of from about 0.001 to 10% (w/w), based on the total weight of the composition. Generally, however for most types of preparations the proportion used will be within the range of from about 0.005 to 1% (w/w), such as about 0.01 to 1% (w/w), for example about 0.01 to 0.5% (w/w), based on the total weight of the composition. However, in powders for inhalation the proportion used will generally be within the range of from about 0.1 to 5% (w/w), based on the total weight of the composition.

Generally, compositions suitable for intranasal or inhaled administration may conveniently be formulated as aerosols, solutions, suspensions, drops, gels or dry powders, optionally with one or more pharmaceutically acceptable carriers and/or excipients such as aqueous or non-aqueous vehicles, thickening agents, isotonicity adjusting agents, antioxidants, preservatives and/or co-solvents.

For compositions suitable for intranasal or inhaled administration, the compound of formula (I) or a pharmaceutically acceptable salt thereof may typically be in a particle-size-reduced form, which may be prepared by conventional techniques, for example, micronisation, milling and/or microfluidisation. Generally, the size-reduced (e.g. micronised) compound of formula (I) or a pharmaceutically acceptable salt thereof can be defined by a D₅₀ value of about 0.5 to 10 microns, for example of about 1 to 10 microns, such as of about 2 to 10 microns (for example as measured using laser diffraction).

In one embodiment, compositions comprising a compound of formula (I) or a pharmaceutically acceptable salt thereof are suitable for intranasal administration. Intranasal compositions comprising a compound of formula (I) or a pharmaceutically acceptable salt thereof may permit the compound(s) to be delivered to all areas of the nasal cavities (the target tissue) and further, may permit the compound(s) to remain in contact with the target tissue for longer periods of time. A suitable dosing regime for intranasal compositions would be for the patient to inhale slowly through the nose subsequent to the nasal cavity being cleared. During inhalation the composition would be administered to one nostril while the other is manually compressed. This procedure would then be repeated for the other nostril. Typically, one or two administrations per nostril would be administered by the above procedure up to two or three times each day, ideally once daily. Of particular interest are intranasal compositions suitable for once daily administration.

The intranasal compositions containing a compound of formula (I) or a pharmaceutically acceptable salt thereof may be in the form of an aqueous suspension and/or an aqueous solution. Partial suspensions and/or partial solutions are encompassed within the scope of the present invention. Compositions comprising one compound which is in solution and another compound which is in suspension are also included within the scope of the present invention.

Intranasal compositions may optionally contain one or more suspending/thickening agents, one or more preservatives, one or more wetting agents and/or one or more isotonicity adjusting agents as desired. Compositions suitable for intranasal administration may optionally further contain other excipients, such as antioxidants (for example sodium metabisulphite), taste-masking agents (such as menthol) and sweetening agents (for example dextrose, glycerol, saccharin and/or sorbitol).

The skilled person would readily appreciate that some excipients may perform more than one function, depending on the nature and number of excipients used in the composition and the particular properties of the therapeutic compound(s) and other carriers and/or excipients contained therein.

The suspending/thickening agent, if included, will typically be present in the intranasal composition in an amount of between about 0.1 and 5% (w/w), such as between about 1.5% and 2.4% (w/w), based on the total weight of the composition. Examples of suspending/thickening agents include, but are not limited to Avicel® (microcrystalline cellulose and carboxymethylcellulose sodium), carboxymethylcellulose sodium, veegum, tragacanth, bentonite, methylcellulose xanthan gum, carbopol and polyethylene glycols. Suspending/thickening agents may also be included in compositions suitable for inhaled, ocular and oral administration as appropriate.

For stability purposes, intranasal compositions comprising a compound of formula (I) or a pharmaceutically acceptable salt thereof may be protected from microbial or fungal contamination and growth by inclusion of a preservative. Examples of pharmaceutically acceptable anti-microbial agents or preservatives may include quaternary ammonium compounds (e.g. benzalkonium chloride, benzethonium chloride, cetrimide, myristal picolinium chloride, lauralkonium chloride and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium ethylenediaminetetraacetate (EDTA) and other anti-microbial agents such as chlorhexidine, chlorocresol, sorbic acid and its salts (such as potassium sorbate) and polymyxin. Examples of pharmaceutically acceptable anti-fungal agents or preservatives include, but are not limited to sodium benzoate, sorbic acid, sodium propionate, methyl paraben, ethyl paraben, propyl paraben and butyl paraben. The preservative, if included, may be present in an amount of between about 0.001 and 1% (w/w), such as about 0.015% (w/w), based on the total weight of the composition. Preservatives may be included in compositions suitable for other routes of administration as appropriate.

Compositions which contain a suspended medicament may include a pharmaceutically acceptable wetting agent which functions to wet the particles of medicament to facilitate dispersion thereof in the aqueous phase of the composition. Typically, the amount of wetting agent used will not cause foaming of the dispersion during mixing. Examples of wetting agents include, but are not limited to fatty alcohols, esters and ethers, such as polyoxyethylene (20) sorbitan monooleate (Polysorbate 80) macrogol ethers and poloxamers. The wetting agent may be present in intranasal compositions in an amount of between about 0.001 and 0.05% (w/w), for example about 0.025% (w/w), based on the total weight of the composition. Wetting agents may be included in compositions suitable for other routes of administration, e.g. for inhaled and/or ocular administration, as appropriate.

An isotonicity adjusting agent may be included to achieve isotonicity with body fluids e.g. fluids of the nasal cavity, resulting in reduced levels of irritancy. Examples of isotonicity adjusting agents include, but are not limited to sodium chloride, dextrose, xylitol and calcium chloride. An isotonicity adjusting agent may be included in intranasal compositions in an amount of between about 0.1 and 10% (w/w), for example between about 4.5 to 5.5% (w/w), such as about 5.0% (w/w), based on the total weight of the composition. Isotonicity adjusting agents may also be included in compositions suitable for other routes of administration, for example in compositions suitable for inhaled, ocular, oral liquid and parenteral administration, as appropriate.

One or more co-solvent(s) may be included to aid solubility of the active compound(s) and/or other excipients. Examples of pharmaceutically acceptable co-solvents include, but are not limited to, propylene glycol, dipropylene glycol, ethylene glycol, glycerol, ethanol, polyethylene glycols (for example PEG300 or PEG400) and methanol. The co-solvent(s), if present, may be included in an amount of from about 0.05 to 20% (w/w), such as from about 1.5 to 17.5% (w/w), or from about 1.5 to 7.5% (w/w), or from about 0.05% to 0.5% (w/w) based on the total weight of the composition. Co-solvents may also be included in compositions suitable for other routes of administration, as appropriate.

Further, the intranasal compositions comprising a compound of formula (I) or a pharmaceutically acceptable salt thereof may be buffered by the addition of suitable buffering agents such as sodium citrate, citric acid, trometarol, phosphates such as disodium phosphate (for example the dodecahydrate, heptahydrate, dihydrate and anhydrous forms) or sodium phosphate and mixtures thereof. Buffering agents may also be included in compositions suitable for other routes of administration as appropriate.

Compositions for administration topically to the nose (for example, for the treatment of rhinitis) or lung, include pressurised aerosol compositions and aqueous compositions delivered to the nasal cavities by pressurised pump. Compositions which are non-pressurised and adapted to be administered topically to the nasal cavity are of particular interest. Suitable compositions contain water as the diluent or carrier for this purpose. Aqueous compositions for administration to the lung or nose may be provided with conventional excipients such as buffering agents, tonicity modifying agents and the like. Aqueous compositions may also be administered to the nose by nebulisation.

A fluid dispenser may typically be used to deliver a fluid composition to the nasal cavities. The fluid composition may be aqueous or non-aqueous, but typically aqueous. Such a fluid dispenser may have a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity. A fluid dispenser of the aforementioned type is described and illustrated in WO05/044354 the entire content of which is hereby incorporated herein by reference. The dispenser has a housing which houses a fluid discharge device having a compression pump mounted on a container for containing a fluid composition. The housing has at least one finger-operable side lever which is movable inwardly with respect to the housing to cam the container upwardly in the housing to cause the pump to compress and pump a metered dose of the composition out of a pump stem through a nasal nozzle of the housing. In one embodiment, the fluid dispenser is of the general type illustrated in FIGS. 30-40 of WO05/044354.

Aqueous compositions containing a compound of formula (I) or a pharmaceutically acceptable salt thereof may also be delivered by a pump as disclosed in WO2007/138084, for example as disclosed with reference to FIGS. 22-46 thereof, or as disclosed in GB0723418.0, for example as disclosed with reference to FIGS. 7-32 thereof, both of which prior patent applications are incorporated herein by reference in their entirety. The pump may be actuated by an actuator as disclosed in FIGS. 1-6 of said GB0723418.0.

In one embodiment, there is provided an intranasal composition comprising a compound of formula (I) or a pharmaceutically acceptable salt thereof. In another embodiment, such an intranasal composition is benzalkonium chloride-free.

Inhaled administration involves topical administration to the lung, such as by aerosol or dry powder composition.

Aerosol compositions suitable for inhaled administration may comprise a solution or fine suspension of the compound in a pharmaceutically acceptable aqueous or non-aqueous solvent. Aerosol compositions suitable for inhalation can be either a suspension or a solution and generally contain a compound of formula (I) or a pharmaceutically acceptable salt thereof and a suitable propellant such as a fluorocarbon or hydrogen-containing chlorofluorocarbon or mixtures thereof, such as hydrofluoroalkanes, e.g. 1,1,1,2-tetrafluoroethane, 1,1,1,2,3,3,3-heptafluoro-n-propane or a mixture thereof. The aerosol composition may optionally contain additional excipients well known in the art such as surfactants or cosolvents. Examples of surfactants include, but are not limited to oleic acid, lecithin, an oligolactic acid or derivative e.g. as described in WO94/21229 and WO98/34596. An example of a cosolvent includes, but is not limited to ethanol. Aerosol compositions may be presented in single or multidose quantities in sterile form in a sealed container, which may take the form of a cartridge or refill for use with an atomising device or inhaler. Alternatively, the sealed container may be a unitary dispensing device such as a single dose nasal inhaler or an aerosol dispenser fitted with a metering valve (metered dose inhaler), which is intended for disposal once the contents of the container have been exhausted.

Dry powder inhalable compositions may take the form of capsules and cartridges of, for example, gelatine, or blisters of, for example, laminated aluminium foil, for use in an inhaler or insufflator. Such compositions may be formulated comprising a powder mix of a compound of formula (I) or a pharmaceutically acceptable salt thereof and a suitable powder base such as lactose or starch.

Optionally, for dry powder inhalable compositions, a composition suitable for inhaled administration may be incorporated into a plurality of sealed dose containers (e.g. comprising the dry powder composition) mounted longitudinally in a strip or ribbon inside a suitable inhalation device. The container is rupturable or peel-openable on demand and the dose of e.g. the dry powder composition may be administered by inhalation via the device such as the DISKUS™ device, marketed by GlaxoSmithKline. The DISKUS™ inhalation device is for example described in GB 2242134 A, and in such a device, at least one container for the composition in powder form (the container or containers may, for example, be a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the composition in powder form from the opened container.

Aerosol compositions are typically arranged so that each metered dose or “puff” of aerosol contains about 20 μg-2000 μg, particularly about 20 μg-500 μg of a compound of formula (I) or a pharmaceutically acceptable salt thereof. Administration may be once daily or several times daily, for example 2, 3, 4 or 8 times, giving for example 1, 2 or 3 doses each time. The overall daily dose with an aerosol will be within the range of about 100 μg-10 mg, such as between about 200 μg-2000 μg. The overall daily dose and the metered dose delivered by capsules and cartridges in an inhaler or insufflator will generally be double those with aerosol compositions.

In another embodiment, there is provided a composition comprising a compound of formula (I) or a pharmaceutically acceptable salt thereof which is suitable for epicutaneous administration. An epicutaneous composition to be applied to the affected area e.g. the skin, by one or more application per day, may be in the form of, for example, an ointment, a cream, an emulsion, a lotion, a foam, a spray, an aqueous gel, or a microemulsion. Such compositions may optionally contain one or more solubilising agents, skin-penetration-enhancing agents, surfactants, fragrances, preservatives or emulsifying agents.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents. Lotions may be formulated with an aqueous or oily base and will in general also contain one or more emulsifying agents, stabilising agents, dispersing agents, suspending agents or thickening agents.

In another embodiment, there is provided a composition comprising a compound of formula (I) or a pharmaceutically acceptable salt thereof which is suitable for ocular administration. Such compositions may optionally contain one or more suspending agents, one or more preservatives, one or more wetting/lubricating agents and/or one or more isotonicity adjusting agents. Examples of ophthalmic wetting/lubricating agents may include cellulose derivatives, dextran 70, gelatin, liquid polyols, polyvinyl alcohol and povidone such as cellulose derivatives and polyols.

In another embodiment, there is provided a composition comprising a compound of formula (I) or a pharmaceutically acceptable salt thereof which is suitable for oral administration. Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

In another embodiment, there is provided a composition comprising a compound of formula (I) or a pharmaceutically acceptable salt thereof which is suitable for parenteral administration. Fluid unit dosage forms suitable for parenteral administration may be prepared utilising a compound of formula (I) or pharmaceutically acceptable salt thereof and a sterile vehicle which may be aqueous or oil based. The compound, depending on the vehicle and concentration used, may be either suspended or dissolved in the vehicle. In preparing solutions, the compound may be dissolved for injection and filter sterilised before filling into a suitable vial or ampoule and sealing. Optionally, adjuvants such as a local anaesthetic, preservatives and buffering agents may be dissolved in the vehicle. To enhance the stability, the composition may be frozen after filling into the vial and the water removed under vacuum. The lyophilised parenteral composition may be reconstituted with a suitable solvent just prior to administration. Parenteral suspensions may be prepared in substantially the same manner, except that the compound is suspended in the vehicle instead of being dissolved, and sterilisation cannot be accomplished by filtration. The compound may be sterilised by exposure to ethylene oxide before suspension in a sterile vehicle. A surfactant or wetting agent may be included in the composition to facilitate uniform distribution of the compound.

The compounds and pharmaceutical compositions according to the invention may also be used in combination with or include one or more other (e.g. one or two) therapeutic agents, for example other antihistaminic agents for example H4 or H3 receptor antagonists, anticholinergic agents, anti-inflammatory agents such as corticosteroids (e.g. fluticasone propionate, fluticasone furoate, beclomethasone dipropionate, mometasone furoate, triamcinolone acetonide, budesonide and the steroid disclosed in WO02/12265); or non-steroidal anti-inflammatory drugs (NSAIDs) (e.g. sodium cromoglycate, nedocromil sodium), PDE-4 inhibitors, leukotriene antagonists, lipoxygenase inhibitors, chemokine antagonists (e.g. CCR3, CCR1, CCR2, CCR4, CCR8, CXCR1, CXCR2), IKK antagonists, iNOS inhibitors, tryptase and elastase inhibitors, beta-2 integrin antagonists and adenosine 2a agonists; or beta adrenergic agents (e.g. salmeterol, salbutamol, formoterol, fenoterol, terbutaline, and the beta agonists described in WO 02/66422, WO 02/270490, WO02/076933, WO03/024439 and WO03/072539 and salts thereof); or antiinfective agents e.g. antibiotic agents and antiviral agents.

It will be clear to a person skilled in the art that, where appropriate, the other therapeutic agent(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic agent. It will be clear also that where appropriate, the therapeutic agents may be used in optically pure form.

There is provided, in another embodiment, a combination comprising a compound of formula (I) or a pharmaceutically acceptable salt thereof and one or more (such as one or two, e.g. one) other therapeutically active agents, optionally with one or more pharmaceutically acceptable carriers and/or excipients.

In another embodiment, there is provided a combination comprising a compound of formula (I) or a pharmaceutically acceptable salt thereof and an H3 and/or H4 antagonist.

Other histamine receptor antagonists which may be used alone, or in combination with an H1 receptor antagonist include antagonists (and/or inverse agonists) of the H4 receptor, for example, the compounds disclosed in Jablonowski et al., J. Med. Chem. 46:3957-3960 (2003), and antagonists (and/or inverse agonists) of the H3 receptor, for example the compounds described in WO2004/035556, the compounds described in WO2006/125665 and the compounds described in WO2006/090142.

In another embodiment, there is provided a combination comprising a compound of formula (I) or a pharmaceutically acceptable salt thereof and a β₂-adrenoreceptor agonist.

Examples of β₂-adrenoreceptor agonists include salmeterol (which may be a racemate or a single enantiomer, such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulfate salt or free base of salbutamol or the fumarate salt of formoterol. In one embodiment, combinations containing a compound of formula (I) may include longer-acting β₂-adrenoreceptor agonists, for example, compounds which provide effective bronchodilation for about 12 h or longer.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxyl methyl)phenol;
4-{(1R)-2-[(6-{4-[3-(cyclopentylsulfonyl)phenyl]butoxy}hexyl)amino]-1-hydroxyethyl}-2-(hydroxylmethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulfuric, hydrochloric, fumaric, hydroxynaphthoic (for example 1- or 3-hydroxy-2-naphthoic), cinnamic, substituted cinnamic, triphenylacetic, sulfamic, sulfanilic, naphthaleneacrylic, benzoic, 4-methoxybenzoic, 2- or 4-hydroxybenzoic, 4-chlorobenzoic and 4-phenylbenzoic acid.

In another embodiment, there is provided a combination comprising a compound of formula (I) or a pharmaceutically acceptable salt thereof and an anti-inflammatory agent.

Anti-inflammatory agents include corticosteroids. Suitable corticosteroids which may be used in combination with the compounds of formula (I) are those oral and inhaled corticosteroids and their pro-drugs which have anti-inflammatory activity. Examples include methyl prednisolone, prednisolone, dexamethasone, fluticasone propionate, 6α,9α-difluoro-1,6-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Corticosteroids of particular interest may include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyano methylester, 6α,9α-difluoro-11β-hydroxy-16α-methyl-17α-(1-methycyclopropylcarbonyl) oxy-3-oxo-androsta-1,4-diene-17β-carbothioic acid S-fluoromethyl ester and mometasone furoate. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) or mometasone furoate.

There is provided, in a further embodiment, a combination comprising a compound of formula (I) or a pharmaceutically acceptable salt thereof, together with a corticosteroid, such as fluticasone propionate or 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) or mometasone furoate. Such combinations may be of particular interest for intranasal administration.

In another embodiment, there is provided a combination comprising a compound of formula (I) or a pharmaceutically acceptable salt thereof and a glucocorticoid agonist.

Non-steroidal compounds having glucocorticoid agonism that may possess selectivity for transrepression over transactivation and that may be useful in combination therapy include those covered in the following patent application and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398 and WO06/015870.

Anti-inflammatory agents include non-steroidal anti-inflammatory drugs (NSAID's).

NSAID's include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis (e.g. montelukast), iNOS (inducible nitric oxide synthase) inhibitors (e.g. oral iNOS inhibitors), IKK antagonists, tryptase and elastase inhibitors, beta-2 integrin antagonists and adenosine receptor agonists or antagonists (e.g. adenosine 2a agonists), cytokine antagonists (e.g. chemokine antagonists, such as a CCR1, CCR2, CCR3, CCR4, or CCR8 antagonists) or inhibitors of cytokine synthesis, or 5-lipoxygenase inhibitors. iNOS inhibitors include those disclosed in WO93/13055, WO98/30537, WO02/50021, WO95/34534 and WO99/62875.

In another embodiment there is provided the use of the compounds of formula (I) or a pharmaceutically acceptable salt thereof in combination with a phosphodiesterase 4 (PDE4) inhibitor. The PDE4-specific inhibitor useful in this embodiment may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds which may be of interest include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996.

Other PDE4 inhibitors include AWD-12-281 from Elbion (Hofgen, N. et al., 15th EFMC Int. Symp. Med. Chem., (Sep. 6-10, Edinburgh) 1998, Abst. P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al., Eur. Resp. J. [Ann. Cong. Eur. Resp. Soc. (Sep. 19-23, Geneva) 1998] 1998, 12 (Suppl. 28): Abst P2393); roflumilast (CAS reference No 162401-32-3) and a pthalazinone (WO99/47505) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al., J. Pharmacol. Exp. Ther., 284(1):162, (1998)), and T2585.

Further PDE4 inhibitors which may be of interest are disclosed in the published international patent applications WO04/024728 (Glaxo Group Ltd), WO04/056823 (Glaxo Group Ltd) and WO04/103998 (Glaxo Group Ltd). A particular compound of interest is 6-({3-[(dimethylamino)carbonyl]phenyl}sulfonyl)-8-methyl-4-{[3-(methyloxy)phenyl]amino}-3-quinolinecarboxamide or a pharmaceutically acceptable salt thereof, which is described in International Patent Application WO04/103998.

In another embodiment, there is provided a combination comprising a compound of formula (I) or a pharmaceutically acceptable salt thereof and an anticholinergic agent.

Anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (for example, CAS 28797-61-7), darifenacin (for example, CAS 133099-04-4, or CAS 133099-07-7 for the hydrobromide sold under the name Enablex), oxybutynin (for example, CAS 5633-20-5, sold under the name Ditropan), terodiline (for example, CAS 15793-40-5), tolterodine (for example, CAS 124937-51-5, or CAS 124937-52-6 for the tartrate, sold under the name Detrol), otilonium (for example, as the bromide, CAS 26095-59-0, sold under the name Spasmomen), trospium chloride (for example, CAS 10405-O₂-4) and solifenacin (for example, CAS 242478-37-1, or CAS 242478-38-2, or the succinate also known as YM-905 and sold under the name Vesicare).

Other anticholinergics may be found in WO 2004/012684; WO2004/091482; WO2005/009439; WO2005/009362; WO2005/009440; WO2005/037280; WO2005/037224; WO2005/046586;WO2005/055940; WO2005/055941; WO2005/067537; WO2005/087236; WO2005/086873; WO2005/094835; WO2005/094834; WO2005/094251; WO2005/095407; WO2005/099706; WO2005/104745; WO2005/112644; WO2005/118594; WO2006/005057; WO2006/017768; WO2006/017767; WO2006/050239; WO2006/055553; WO2006/055503; WO2006/065755; WO2006/065788; WO2007/018514; WO2007/018508; WO2007/016650; WO2007/016639; and WO2007/022351.

Other anticholinergic agents include compounds which are disclosed in U.S. patent application 60/487,981, published as WO2005/009439 and those compounds disclosed in U.S. patent application 60/511,009, published as WO2005/037280.

The combinations referred to above may conveniently be presented for use in the form of a pharmaceutical composition and thus pharmaceutical compositions comprising a combination as defined above optionally together with a pharmaceutically acceptable carrier and/or excipient.

The individual compounds of such combinations may be administered either sequentially in separate pharmaceutical compositions as well as simultaneously in combined pharmaceutical compositions. Additional therapeutically active ingredients may be suspended in the composition together with a compound of formula (I). Appropriate doses of known therapeutic agents will be readily appreciated by those skilled in the art.

Compounds of formula (I) may be prepared by the methods described below or by similar methods. Thus the following Intermediates and Examples illustrate the preparation of the compounds of formula (I), and are not to be considered as limiting the scope of the disclosure in any way.

### EXPERIMENTAL SECTION

### Abbreviations

9-BBN: 9-Borabicyclo[3.3.1]nonane
DCM: Dichloromethane
DMF: N,N-dimethyl formamide
EtOAc: Ethyl acetate
h: hours
HATU: 2-(1H-7-Azabenzotriazol-1-yl)-1,1,3,3-tetramethyl uronium hexafluororophosphate
LCMS: Liquid Chromatography-Mass Spectroscopy
MDAP: Mass-Directed Auto-Purification
MeOH: Methanol
MgSO₄: Magnesium sulfate
Min: minutes
Na₂SO₄: Sodium sulfate
NMP: N-methylpyrrolidinone
PyBOP: (Benzotriazol-1-yloxy)tripyrrolidinophosphonium hexafluoro phosphate
s.g.: specific gravity (gml⁻¹)
TBTU: O-(Benzotriazol-1-yl)-N,N,N¹,N¹-tetramethyluronium tetrafluoroborate
TFA: Trifluoroacetic acid
THF: Tetrahydrofuran

### General Experimental Procedures

Flash silica gel refers to Merck Art No. 9385; silica gel refers to Merck Art No. 7734.

SCX cartridges are Ion Exchange SPE columns where the stationary phase is polymeric benzene sulfonic acid. These are used to isolate amines.

SCX2 cartridges are Ion Exchange SPE columns where the stationary phase is polymeric propylsulfonic acid. These are used to isolate amines.

LCMS was conducted on a Supelcosil LCABZ+PLUS column (3.3 cm×4.6 mm ID) eluting with 0.1% formic acid and 0.01 M ammonium acetate in water (solvent A) and 0.05% formic acid 5% water in MeCN (solvent B), using the following elution gradient 0.0-7 min 0% B, 0.7-4.2 min 100% B, 4.2-5.3 min 0% B, 5.3-5.5 min 0% B at a flow rate of 3 mlmin⁻¹. The mass spectra were recorded on a Fisons VG Platform spectrometer using electrospray positive and negative mode (ES+ve and ES−ve).

The Flashmaster II is an automated multi-user flash chromatography system, available from Argonaut Technologies Ltd, which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength UV-detector and two Gilson FC204 fraction-collectors enabling automated peak cutting, collection and tracking.

### Mass Directed Autopreparative (MDAP) HPLC:

Method A was conducted on a Waters FractionLynx system comprising of a Waters 600 pump with extended pump heads, Waters 2700 autosampler, Waters 996 diode array and Gilson 202 fraction collector on a 10 cm×2.54 cm internal diameter ABZ+ column, eluting with 0.1% formic acid in water (solvent A) and 0.1% formic acid in MeCN (solvent B), using an appropriate elution gradient over 15 min at a flow rate of 20 mlmin⁻¹ and detecting at 200-320 nm at room temperature. Mass spectra were recorded on Micromass ZMD mass spectrometer using electro spray positive and negative mode, alternate scans. The software used was MassLynx 3.5 with OpenLynx and FractionLynx options. Method A was used, unless otherwise stated.

Method B was conducted on Agilent 1100 series LC/MSD hardware, using electrospray positive mode (ES+ve) running chemstation 32 purification software on a 21.2 mm×100 mm Zorbax Eclipse XDB-C18 prep HT column (5 μm packing), eluting with 0.1% TFA in water (solvent A) and 0.1% TFA in acetonitrile (solvent B), using an appropriate elution gradient over min at a flow rate of 20 ml/min.

Method C was conducted on Agilent 1100 series LC/MSD hardware, using electrospray positive mode (ES+ve) running chemstation 32 purification software on a 19 mm×100 mm Xbridge prep C18 OBD column (5 μm packing), eluting with ammonium bicarbonate (10 mM) buffered to pH10 with aqueous 0.880 s.g. ammonia, and 0.1% aqueous 0.880 s.g. ammonia, using an appropriate elution gradient over min at a flow rate 20 ml/min.

The ¹H NMR spectra were recorded on a Bruker AV400 operating at 400 MHz. Standard deuterated solvents were used. Tetramethylsilane may have been used as internal standard.

Reactions are routinely monitored by methods well known to those skilled in the art, such as TLC, LCMS and/or HPLC. Such methods are used to assess whether a reaction has gone to completion, and reaction times may be varied accordingly.

Compounds were named using ACD/Name PRO6.02 chemical naming software Advanced Chemistry Developments Inc.; Toronto, Ontario, M5H2L3, Canada.

### Intermediates

### Intermediate 1

### 6-Bromo-8-fluoroquinoline

A solution of concentrated sulphuric acid (63 ml, 820 mmol) in water (49.4 ml) was treated with sodium 3-nitro-benzenesulfonate (commercially available, for example, from Aldrich) (47.9 g, 213 mmol) and glycerol (commercially available, for example, from Fluka) (52 ml, 720 mmol) to give a thick grey suspension. This was heated to 110° C. 4-Bromo-2-fluoroaniline (commercially available, for example, from Fluorochem) (38 g, 200 mmol) was added portion wise over 10 min, during which the temperature rose to 95° C. The reaction was heated to 140° C. and stirred overnight. The reaction mixture was cooled and then poured into water (1000 ml) and basified to pH 7 with aqueous ammonia (0.88 s.g., 190 ml). The brown precipitated that formed was collected by filtration and partially dried. This solid (63 g) was loaded onto a Silica column (1500 ml) and eluted with EtOAc to give the title compound (43.8 g, 96.9%) LCMS RT=2.87 min, ES+ve m/z 226/228 [M+H]⁺.

### Intermediate 2

### 6-Butyl-8-fluoroquinoline

A mixture of 6-bromo-8-fluoroquinoline (for example, as prepared for Intermediate 1) (24 g, 106 mmol) in DMF (150 ml) was treated under nitrogen with potassium carbonate (33 g, 240 mmol), tributylborane solution in THF (commercially available, for example, from Aldrich) (1M, 200 ml) and [1,1″-bis(diphenylphosphino) ferrocene palladium (II)] chloride (1 g, 1.2 mmol). The resulting mixture was stirred under nitrogen and heated at 75° C. overnight. The mixture was allowed to cool, diluted with water and extracted with EtOAc (×3). The combined organic layers were filtered through a frit to remove any insoluble material and the filtrate was washed with water. The organic layer was dried (MgSO₄), and the filtrate evaporated to dryness. The residue was purified by flash chromatography twice eluting with DCM-EtOAc (1:0 to 2:1) to afford the title compound (14.47 g, 67%). LCMS RT=3.38 min, ES+ve m/z 204 [M+H]⁺.

### Intermediate 3

### 1,1-Dimethylethyl 4-[(6-butyl-8-quinolinyl)oxy]-1-piperidinecarboxylate

A solution of 6-butyl-8-fluoroquinoline (for example, as prepared, for Intermediate 2) (14.4 g, 70.9 mmol) in NMP (20 ml) was added to a mixture of tert-butyl-4-hydroxy-1-piperidinecarboxylate (commercially available, for example, from Aldrich) (21.6 g, 108 mmol) and sodium tert-butoxide (10.4 g, 108 mmol) in NMP (75 ml), and the resulting mixture was stirred at 140° C. for 90 min and then allowed to cool overnight. The reaction mixture was treated with ammonium chloride solution and extracted with EtOAc (×2). The combined organic extracts were washed with water, dried (MgSO₄), filtered and evaporated to dryness. The residue was purified by flash chromatography eluting with DCM-EtOAc (1:0 to 1:1) and then by flashmaster chromatography eluting with DCM-EtOAc (1:0 to 3:1) over 40 min give the title compound (22 g). LCMS RT=3.49 min, ES+ve m/z 385 [M+H]⁺

### Intermediate 4

### 1,1-Dimethylethyl (3R)-3-[(6-butyl-8-quinolinyl)oxy]-1-pyrrolidinecarboxylate

This was prepared in an analogous manner to Intermediate 3, using N-tert-butoxycarbonyl-(R)-(−)-3-pyrrolidinol (commercially available, for example, from Aldrich) instead of 1,1-dimethylethyl 4-hydroxy-1-piperidinecarboxylate. The reaction time was 3 h instead of 1.5 h. LCMS RT=3.56 min, ES+ve m/z 371 (M+H)⁺.

### Intermediate 5

### 1,1-Dimethylethyl (3S)-3-[(6-butyl-8-quinolinyl)oxy]-1-pyrrolidinecarboxylate

The title compound was prepared in an analogous manner to Intermediate 3, using N-tert-butoxycarbonyl-(S)-(−)-3-pyrrolidinol (commercially available, for example, from Aldrich) instead of 1,1-dimethylethyl 4-hydroxy-1-piperidinecarboxylate. The reaction time was 3 h instead of 1.5 h. LCMS RT=3.56 min, ES+ve m/z 371 (M+H)⁺.

### Intermediate 6

### 6-Butyl-8-(4-piperidinyloxy) quinoline

1,1-Dimethylethyl 4-[(6-butyl-8-quinolinyl)oxy]-1-piperidinecarboxylate (for example, as prepared for Intermediate 3) (21.5 g, 56 mmol) was dissolved in DCM (50 ml) and TFA (50 ml) was added very slowly. The mixture was stirred at room temperature for 1 h. The solvent was evaporated to dryness and the residue treated with saturated aqueous sodium carbonate solution. The mixture was extracted with EtOAc (×2), washed with water, and dried (MgSO₄). The drying agent was removed by filtration and the filtrate was evaporated to dryness (22 g). This was still a TFA salt and thus was re-dissolved in EtOAc, washed with aqueous sodium carbonate, water, and dried (MgSO₄). The drying agent was removed by filtration and the filtrate was evaporated to dryness to afford the title compound (15.9 g). LCMS RT=2.45 min, ES+ve m/z 285 [M+H]⁺.

### Intermediate 7

### 6-Butyl-8-[(3R)-3-pyrrolidinyloxy]quinoline

The title compound was prepared in an analogous manner to Intermediate 6, using 1,1-dimethylethyl (3R)-3-[(6-butyl-8-quinolinyl)oxy]-1-pyrrolidinecarboxylate (for example, as prepared for Intermediate 4) and 4 M hydrogen chloride in 1,4-dioxane instead of TFA, for 45 min. LCMS RT=2.46 min, ES+ve m/z 271 (M+H)⁺.

### Intermediate 8

### 6-Butyl-8-[(3S)-3-pyrrolidinyloxy]quinoline

The title compound was prepared in an analogous manner to Intermediate 6, using 1,1-dimethylethyl (3S)-3-[(6-butyl-8-quinolinyl)oxy]-1-pyrrolidinecarboxylate (for example, as prepared for Intermediate 5) and 4 M hydrogen chloride in 1,4-dioxane instead of TFA, for 45 min. LCMS RT=2.48 min, ES+ve m/z 271 (M-FH)⁺.

### Intermediate 9

### 1,1-Dimethylethyl 4-[(6-bromo-8-quinolinyl)oxy]-1-piperidinecarboxylate

6-Bromo-8-fluoroquinoline (for example, as prepared for Intermediate 1) (2 g, 8.8 mmol), 1,1-dimethylethyl 4-hydroxy-1-piperidinecarboxylate (commercially available, for example, from Aldrich) (2.6 g, 13.2 mmol), sodium tert-butoxide (1.3 g, 13.2 mmol) and NMP (20 ml) were combined and heated under microwave conditions in two equal batches at 140° C. for 12 min. The batches were combined and the mixture was partitioned between EtOAc and water. The aqueous phase was extracted with EtOAc (×3) and the combined organic layers were dried over a hydrophobic frit and the solvent evaporated to give the title compound (4.1 g). LCMS RT=3.45 min, ES+ve m/z 407/409 [M+H]⁺.

### Intermediate 10

### 6-Pentyl-8-(4-piperidinyloxy)quinoline

1-Pentene (commercially available, for example, from Aldrich) (0.034 ml, 0.32 mmol) in 0.5 M 9-BBN in THF (1.3 ml, 0.95 mmol) was stirred in a sealed tube for 3 h at 20° C. 1 M aqueous sodium hydroxide solution (0.21 ml, 0.32 mmol) was added and stirring continued for a further 30 min. 1,1′-Bis(diphenylphosphino)ferrocene palladium dichloride (0.026 g, 0.042 mmol) and 1,1-dimethylethyl 4-[(6-bromo-8-quinolinyl)oxy]-1-piperidinecarboxylate (for example, as prepared for Intermediate 9) (0.85 g, 0.21 mmol) in THF (0.5 ml) were added and the reaction was stirred for ten days at 20° C. The reaction mixture was diluted with EtOAc (50 ml) and washed with water (2×50 ml). The organic layer was extracted with 5 M aqueous hydrochloric acid (50 ml). The acidic layer was washed with diethyl ether (3×30 ml) and left to stand for 30 min. It was neutralised with aqueous sodium hydroxide solution and extracted with EtOAc (3×50 ml). The organics were combined and dried over a hydrophobic frit and evaporated. The residue was applied to a SCX cartridge (5 g), washed with DCM-MeOH (9:1), and the product was eluted with DCM-1 M ammonia in MeOH (9:1). The appropriate fractions were combined and evaporated to give the title compound (0.056 g, 89%). LCMS RT=2.32 min, ES+ve m/z 299 [M+H]⁺.

### Intermediate 11

### 6-Chloro-8-fluoroquinoline

A mixture of 4-chloro-2-fluoroaniline (commercially available, for example, from Aldrich) (6.0 g), p-chloranil (commercially available, for example, from Aldrich) (10 g), 1-butanol (20 ml), and 37% aqueous hydrochloric acid (10 ml) was heated to 120° C. under nitrogen. A solution of acrolein (commercially available, for example, from Aldrich) (4.5 g) in 1-butanol (10 ml) was added dropwise over 15 min, to elicit a gentle reflux. The resulting dark mixture was heated at 120° C. for 2 min post-addition, cooled and added to 1 N aqueous hydrochloric acid (250 ml). The resulting suspension was filtered through hyflo and the filtered solid was washed with 2 N aqueous hydrochloric acid (2×50 ml). The combined acidic solution was washed with diethyl ether (2×200 ml) and basified with 2 N aqueous sodium hydroxide. The basic solution was allowed to cool and was extracted with DCM (3×300 ml). The dried (Na₂SO₄) extract was evaporated to give a dark brown solid. The solid was purified on a silica cartridge (100 g) eluting with DCM through to DCM-EtOAc (1:1) to give the title compound as a pale yellow solid (2.7 g). LCMS RT=2.79 min, ES+ve m/z 182 (M-FH)⁺.

### Intermediate 12

### 1,1-Dimethylethyl 4-[(6-chloro-8-quinolinyl)oxy]-1-piperidinecarboxylate

A mixture of 6-chloro-8-fluoroquinoline (for example, as prepared for Intermediate 11) (2.5 g), 1,1-dimethylethyl 4-(methyloxy)-1-piperidinecarboxylate (commercially available, for example, from Aldrich) (4 g), sodium tert-butoxide (2 g) and NMP (8 ml) was heated to 140° C. for 1 h. The mixture was cooled, added to water (250 ml) and extracted with EtOAc (3×150 ml). Brine (50 ml) was added to help layer separation. The dried (Na₂SO₄) extract was evaporated and the residue was purified on a silica cartridge (100 g) eluting with cyclohexane through cyclohexane-EtOAc (1:1) to give the title compound as a yellow gum (2.6 g). The gum later solidified to give a beige solid. LCMS RT=3.46 min, ES+ve m/z 363 (M+H)⁺.

### Intermediate 13

### 1,1-Dimethylethyl 4-[(6-hexyl-8-quinolinyl)oxy]-1-piperidinecarboxylate

Hexylmagnesium bromide (2 M in diethyl ether) (commercially available, for example, from Aldrich) (1.65 ml) was added dropwise to a solution of 1,1-dimethylethyl 4-[(6-chloro-8-quinolinyl)oxy]-1-piperidinecarboxylate (for example, as prepared for Intermediate 12) (600 mg) and iron(III) acetylacetanoate (27 mg) in THF (8 ml) and NMP (0.8 ml) at 0° C. under nitrogen. The mixture was stirred for 1 h at room temperature and further iron catalyst (21 mg) and hexylmagnesium bromide (1 ml) were added. The solution was stirred for 15 min at room temperature, added to saturated aqueous ammonium chloride (50 ml) and extracted with EtOAc (3×30 ml). The dried (Na₂SO₄) extract was evaporated and the residue was purified on a column of silica (50 g) eluting with cyclohexane through to cyclohexane-EtOAc (1:1) to give the title compound as a yellow gum (500 mg). LCMS RT=3.79 min, ES+ve m/z 413 (M+H)⁺.

### Intermediate 14

### 6-Hexyl-8-(4-piperidinyloxy)quinoline

A solution of 1,1-dimethylethyl 4-[(6-hexyl-8-quinolinyl)oxy]-1-piperidinecarboxylate (for example, as prepared for Intermediate 13) (500 mg) in DCM (3 ml) was treated with TFA (1 ml) and left for 45 min at room temperature. The solution was evaporated and the residue was dissolved in DCM and passed through an aminopropyl cartridge (10 g). The compound was eluted with DCM-MeOH (4:1, 2 column volumes) and the resulting solution was evaporated to give the title compound as a yellow gum (300 mg). LCMS RT=2.63 min, ES+ve m/z 313 (M+H)⁺.

### Intermediate 15

### 1,1-Dimethylethyl 4-({4-[(6-butyl-8-quinolinyl)oxy]-1-piperidinyl}methyl)-1-piperidine carboxylate

To a solution of 6-butyl-8-(4-piperidinyloxy)quinoline (for example, as prepared for Intermediate 6) (400 mg, 1.41 mmol) in DCM (9.0 ml) were added 1,1-dimethylethyl 4-formyl-1-piperidinecarboxylate (commercially available, for example, from Aldrich) (599.7 mg, 2.81 mmol), acetic acid (80.63 μl, 1.41 mmol) and sodium triacetoxyborohydride (596.3 mg, 2.81 mmol). The mixture was left overnight. Additional quantities of 1,1-dimethylethyl 4-formyl-1-piperidinecarboxylate (149.9 mg, 0.7 mmol) and sodium triacetoxyborohydride (149.1 mg, 0.7 mmol) were added and the mixture was left for 4 h. The reaction mixture was filtered and the filtrate was purified on an aminopropyl ion-exchange cartridge (10 g) that was eluted with chloroform (25 ml), then 10% MeOH in EtOAc (25 ml) and finally MeOH (25 ml). The solvent was evaporated from appropriate fractions to afford the title compound (395.6 mg). LCMS RT=2.69 min, ES+ve m/z 482 [M+H]⁺.

### Intermediate 16

### 6-Butyl-8-{[1-(4-piperidinylmethyl)-4-piperidinyl]oxy}quinoline

1,1-Dimethylethyl-4-({4-[(6-butyl-8-quinolinyl)oxy]-1-piperidinyl}methyl)-1-piperidine carboxylate (for example, as prepared for Intermediate 15) (395.6 mg, 0.82 mmol) was dissolved in DCM (4 ml) and TFA (2 ml) was added to the solution. The reaction mixture was stirred under nitrogen for 1 h. The solvent was evaporated from the reaction mixture giving a residue that was treated with toluene (15 ml) then re-evaporated. This process was repeated. The residue was dissolved in MeOH (10 ml) and loaded onto an SCX ion-exchange cartridge (10 g). The column was washed with MeOH (2 column volumes) and the product was eluted with 2 N ammonia in MeOH (3 column volumes). The solvent was evaporated from the ammoniacal fractions to afford the title compound (279.6 mg). LCMS RT=2.04 min, ES+ve m/z 382 [M+H]⁺.

### Intermediate 17

### 1,1-Dimethylethyl 4-(2-{4-[(6-butyl-8-quinolinyl)oxy]-1-piperidinyl}ethyl)-1-piperidine carboxylate

To a solution of 6-butyl-8-(4-piperidinyloxy) quinoline (for example, as prepared for Intermediate 6) (387.9 mg, 1.36 mmol) in DCM (9.0 ml) was added N-Boc-piperidinyl-4-acetaldehyde (commercially available, for example, from Pharmacore) (617.4 mg, 2.72 mmol), acetic acid (78.2 μl) and sodium triacetoxyborohydride (576.5 mg, 2.72 mmol). The mixture was left overnight, filtered and the filtrate was loaded onto an aminopropyl ion-exchange cartridge (10 g). The cartridge was eluted with chloroform (25 ml), 10% MeOH-EtOAc (25 ml) and MeOH (25 ml). The solvent was evaporated from an appropriate fraction to afford the title compound (132.9 mg). LCMS RT=2.73 min, ES+ve m/z 496 [M+H]⁺. The solvent was evaporated from a further fraction giving a residue that was dissolved in DCM (10 ml) and loaded onto a silica SPE cartridge (20 g). The column was eluted with 2% (2 N ammonia in MeOH) in DCM (150 ml), followed by 5% (2 N ammonia in MeOH) in DCM (100 ml). Appropriate fractions were combined and the solvent was evaporated to afford a further sample of the title compound (370.6 mg). LCMS RT=2.76 min, ES+ve m/z 496 [M+H]⁺.

### Intermediate 18

### 6-Butyl-8-({1-[2-(4-piperidinyl)ethyl]-4-piperidinyl}oxy)quinoline

1,1-Dimethylethyl-4-(2-{4-[(6-butyl-8-quinolinyl)oxy]-1-piperidinyl}ethyl)-1-piperidine carboxylate (for example, as prepared for Intermediate 17) (503.5 mg, 1.02 mmol) was dissolved in DCM (5 ml) and TFA (2.5 ml) was added to the solution. The mixture was stirred under nitrogen for 1 h. The solvent was evaporated from the reaction mixture giving a residue that was treated with toluene (25 ml) and then re-evaporated. This process was repeated. The residue was dissolved in MeOH (5 ml) and loaded onto an SCX ion-exchange cartridge (10 g). The column was washed with MeOH (2 column volumes) and the product was eluted with 2 N ammonia in MeOH (3 column volumes). The solvent was evaporated from the ammonia-containing fractions to afford the title compound (395.9 mg). LCMS RT=2.10 min, ES+ve m/z 396 [M+H]⁺.

### Intermediate 19

### (2-Oxo-3-piperidinyl)methyl methanesulfonate

3-(Hydroxymethyl)-2-piperidinone (prepared as disclosed by R. D. Smith et al., J. Med. Chem., 1981, 24, 104, compound 2a) (129 mg, 1 mmol) was stirred with triethylamine (0.28 ml, 2 mmol) in DCM (2 ml) at room temperature and methanesulfonyl chloride (commercially available, for example, from Aldrich) (0.086 ml, 1.1 mmol) was added under nitrogen. After 2 h, the mixture was partitioned between DCM and water acidified by addition of 2 M hydrochloric acid. The organic layer was washed with water, saturated sodium bicarbonate and water in succession, each time back-extracting with DCM. The combined organic solutions were dried (MgSO₄) and evaporated to dryness to give the title compound (50 mg), LCMS RT=1.19 min, ES+ve m/z 208 (M+H)⁺. All the aqueous layers from the above extractions and washings were combined and extracted with DCM (×3). The combined organic layers were dried (MgSO₄) and evaporated to give further title compound (67 mg), LCMS RT=1.19 min, ES+ve m/z 208 (M+H)⁺

### Intermediate 20

### Mixture of Methyl and Ethyl Esters of 4-{4-[(6-butyl-8-quinolinyl)oxy]-1-piperidinyl}butanoate

6-Butyl-8-(4-piperidinyloxy)quinoline (for example, as prepared for Intermediate 6) (1.14 g, 4.0 mmol) was dissolved in DMF (30 ml). Ethyl 4-bromobutanoate (commercially available, for example, from Aldrich) (0.86 ml, 6.0 mmol) and potassium carbonate (5.05 g, 20.0 mmol) were added. The mixture was heated to 60° C. with stirring under a nitrogen atmosphere overnight. After cooling, the mixture was filtered, and the filtrate was concentrated in vacuo. The residue was dissolved in MeOH, and this solution was applied to a SCX-2 ion exchange cartridge (50 g, pre-conditioned with MeOH). The cartridge was washed with MeOH (3 column volumes), and then eluted with 10% 0.880 s.g. ammonia in MeOH. The relevant basic fractions were concentrated in vacuo. The residue was further purified by chromatography on silica (50 g, eluting with EtOAc-cyclohexane 0-100%, followed by (1% triethylamine in MeOH)-EtOAc, 0-20%). The relevant fractions were concentrated in vacuo to give the title compound as a mixture of methyl and ethyl esters (1.2 g, 78%). LCMS RT=2.56 min, ES+ve m/z 385 (M+H)⁺ for methyl ester and RT=2.69 min, ES+ve m/z 399 (M+H)⁺ for ethyl ester.

Intermediates 21 to 23 were prepared in an analogous manner to Intermediate 20:

### Intermediate 21

### Methyl 2-{4-[(6-butyl-8-quinolinyl)oxy]-1-piperidinyl}acetate

The title compound was prepared by the reaction of 6-butyl-8-(4-piperidinyloxy)quinoline (for example, as prepared for Intermediate 6) and ethyl 2-bromoacetate (commercially available, for example, from Aldrich) LCMS RT=2.66 min, ES+ve m/z 371 (M+H)⁺

### Intermediate 22

### Methyl 3-{4-[(6-butyl-8-quinolinyl)oxy]-1-piperidinyl}propanoate

The title compound was prepared by the reaction of 6-butyl-8-(4-piperidinyloxy)quinoline (for example, as prepared for Intermediate 6) and 3-bromopropanoate (commercially available, for example, from Aldrich). LCMS RT=2.60 min, ES+ve m/z 371 (M+H)⁺.

### Intermediate 23

### Methyl 5-{4-[(6-butyl-8-quinolinyl)oxy]-1-piperidinyl}pentanoate

The title compound was prepared by the reaction of 6-butyl-8-(4-piperidinyloxy)quinoline (for example, as prepared for Intermediate 6) with methyl 5-bromopentanoate (commercially available, for example, from Aldrich). LCMS RT=2.67 min, ES+ve m/z 399 (M+H)⁺.

### Intermediate 24

### Methyl 3-{(3R)-3-[(6-butyl-8-quinolinyl)oxy]-1-pyrrolidinyl}propanoate

To a solution of 6-butyl-8-[(3R)-3-pyrrolidinyloxy]quinoline (for example, as prepared for Intermediate 7) (0.35 g, 1.29 mmol) in THF (2 ml) was added methyl acrylate (commercially available, for example, from Aldrich) (0.4 ml, 4.44 mmol). The solution was heated to 65° C. for 3 h. The solvent was removed in vacuo and the residue purified by silica chromatography (20 g cartridge eluting with 0-30% MeOH in DCM over 30 min). The appropriate fractions were combined and the solvent removed in vacuo to give the title compound as a yellow oil (0.42 g, 91%). LCMS RT=2.86 min, ES+ve m/z 357 (M+H)⁺.

### Intermediate 25

### Ethyl 4-{(3R)-3-[(6-butyl-8-quinolinyl)oxy]-1-pyrrolidinyl}butanoate

The title compound was prepared in an analogous manner to Intermediate 24, using 6-butyl-8-[(3R)-3-pyrrolidinyloxy]quinoline, (for example, as prepared for Intermediate 7), N,N-diisopropylethylamine instead of potassium carbonate, and THF instead of DMF. LCMS RT=3.05 min, ES+ve m/z 385 (M+H)⁺.

### Intermediate 26

### Methyl 3-{(3S)-3-[(6-butyl-8-quinolinyl)oxy]-1-pyrrolidinyl}propanoate

The title compound was prepared in an analogous manner to Intermediate 24, using 6-butyl-8-[(3S)-3-pyrrolidinyloxy]quinoline (for example, as prepared for Intermediate 8). LCMS RT=2.86 min, ES+ve m/z 357 (M+H)⁺.

### Intermediate 27

### Ethyl 4-{(3S)-3-[(6-butyl-8-quinolinyl)oxy]-1-pyrrolidinyl}butanoate

The title compound was prepared in an analogous manner to Intermediate 24, using 6-butyl-8-[(3S)-3-pyrrolidinyloxy]quinoline, (for example, as prepared for Intermediate 8), N,N-diisopropylethylamine instead of potassium carbonate, and THF instead of DMF. LCMS RT=2.98 min, ES+ve m/z 385 (M+H)⁺.

### Intermediate 28

### 4-{4-[(6-Butyl-8-quinolinyl)oxy]-1-piperidinyl}butanoic acid, partial triethylamine salt

The mixture of methyl and ethyl esters of 4-{4-[(6-butyl-8-quinolinyl)oxy]-1-piperidinyl}butanoate (for example, as prepared for Intermediate 20) (1.2 g, 3.1 mmol) was stirred in a mixture of MeOH (6 ml) and 2 M aqueous sodium hydroxide (6 ml) at room temperature for 3 h. The solvent was removed in vacuo, and the residue was dissolved in water (6 ml) and treated with 2 M aqueous hydrochloric acid (7 ml). This mixture was applied to a SCX-2 ion exchange cartridge (50 g, pre-conditioned with MeOH (1 column volume) and water (2 column volumes)). The cartridge was washed with water (1 column volume), and then eluted with 10% triethylamine in MeOH. The relevant basic fractions were concentrated in vacuo to give the title compound as the partial triethylamine salt, also containing approximately 2 equivalents of DCM (1.5 g, 98%). LCMS RT=2.55 min, ES+ve m/z 371 (M+H)⁺.

Intermediates 29 to 35 were prepared in an analogous manner to Intermediate 28:

### Intermediate 29

### 2-{4-[(6-Butyl-8-quinolinyl)oxy]-1-piperidinyl}acetic acid, triethylamine salt

The title compound was prepared from methyl 2-{4-[(6-butyl-8-quinolinyl)oxy]-1-piperidinyl}acetate (for example, as prepared for Intermediate 21) using lithium hydroxide in THF/water (6:1). LCMS RT=2.36 min, ES+ve m/z 343 (M+H)⁺.

### Intermediate 30

### 3-{4-[(6-Butyl-8-quinolinyl)oxy]-1-piperidinyl}propanoic acid, triethylamine Salt

The title compound was prepared from methyl 3-{4-[(6-butyl-8-quinolinyl)oxy]-1-piperidinyl}propanoate (for example, as prepared for Intermediate 22) using aqueous sodium hydroxide in MeOH. LCMS RT=2.46 min, ES+ve m/z 357 (M+H)⁺.

### Intermediate 31

### 5-{4-[(6-Butyl-8-quinolinyl)oxy]-1-piperidinyl}pentanoic acid, triethylamine Salt

The title compound was prepared from methyl 5-{4-[(6-butyl-8-quinolinyl)oxy]-1-piperidinyl}pentanoate (for example, as prepared for Intermediate 23) using lithium hydroxide in THF/water (6:1). LCMS RT=2.56 min, ES+ve m/z 385 (M+H)⁺.

### Intermediate 32

### 3-{(3R)-3-[(6-Butyl-8-quinolinyl)oxy]-1-pyrrolidinyl}propanoic acid

The title compound was prepared using methyl 3-{(3R)-3-[(6-butyl-8-quinolinyl)oxy]-1-pyrrolidinyl}propanoate (for example, as prepared for Intermediate 24). LCMS RT=2.49 min, ES+ve m/z 343 (M+H)⁺.

### Intermediate 33

### 4-{(3R)-3-[(6-Butyl-8-quinolinyl)oxy]-1-pyrrolidinyl}butanoic acid

The title compound was prepared using ethyl 4-{3R)-3-[(6-butyl-8-quinolinyl)oxy]-1-pyrrolidinyl}butanoate (for example, as prepared for Intermediate 25). LCMS RT=2.62 min, ES+ve m/z 357 (M+H)⁺.

### Intermediate 34

### 3-{(3S)-3-[(6-Butyl-8-quinolinyl)oxy]-1-pyrrolidinyl}propanoic acid

The title compound was prepared using methyl 3-{(3S)-3-[(6-butyl-8-quinolinyl)oxy]-1-pyrrolidinyl}propanoate (for example, as prepared for Intermediate 26). LCMS RT=2.46 min, ES+ve m/z 343 (M+H)⁺.

### Intermediate 35

### 4-{(3S)-3-[(6-Butyl-8-quinolinyl)oxy]-1-pyrrolidinyl}butanoic acid

The title compound was prepared using ethyl 4-{(3S)-3-[(6-butyl-8-quinolinyl)oxy]-1-pyrrolidinyl}butanoate (for example, as prepared for Intermediate 27). LCMS RT=2.60 min, ES+ve m/z 357 (M+H)⁺.

### Intermediate 36

### Trans-4-({4-[(6-butyl-8-quinolinyl)oxy]-1-piperidinyl}methyl)cyclohexane carboxylic acid

To a solution of 6-butyl-8-(4-piperidinyloxy)quinoline (for example, as prepared for Intermediate 6) (0.6 g, 2.1 mmol) in DCM (15 ml) was added 4-formylcyclohexanecarboxylic acid (commercially available, for example, from Peakdale) (0.56 g, 3.59 mmol) and then acetic acid (0.205 ml, 3.59 mmol). The slight suspension was stirred vigorously for 5 min. To this suspension was added sodium triacetoxyborohydride (0.760 g, 3.59 mmol) and the mixture was stirred at ambient temperature overnight. The mixture was diluted with MeOH (5 ml). The solvent was removed in vacuo and the residue applied to an amino propyl ion exchange cartridge (50 g, pre-conditioned with MeOH). The cartridge was washed with MeOH (2 column volumes) and then eluted with 10% 0.880 s.g. ammonia in MeOH (2 column volumes). The basic fractions were concentrated in vacuo to leave a colourless gum (0.5 g). Product was detected in the MeOH fractions. The MeOH fractions were combined and concentrated in vacuo to leave a yellow gum (1 g). The crude product from both the basic and the MeOH fractions were combined and purified by silica chromatography (2×50 g cartridges, eluting with 0-50% MeOH-DCM over 40 min). The appropriate fractions were combined and the solvent removed in vacuo to give the title compound as a colourless gum (0.396 g, 44%). LCMS RT=2.71 min, ES+ve m/z 425 (M+H)⁺.

### Intermediate 37

### 4-({4-[(6-Hexyl-8-quinolinyl)oxy]-1-piperidinyl}methyl)cyclohexanecarboxylic acid

A solution of 6-hexyl-8-(4-piperidinyloxy)quinoline (for example, as prepared for Intermediate 14) (280 mg, 0.9 mmol), and 4-formylcyclohexanecarboxylic acid (commercially available, for example, from Peakdale) (167 mg, 1.1 mmol) in DCM (3 ml) was treated with sodium triacetoxyborohydride (222 mg, 1.05 mmol) at room temperature under nitrogen. The mixture was stirred at room temperature for 4 h and was allowed to stand for 16 h. The mixture was diluted with DCM (7 ml) and treated with pH 7 phosphate buffer (10 ml). The resulting emulsion was filtered through hyflo and the organic phase separated. The aqueous phase was further extracted with DCM (2×10 ml). The combined organic extracts were dried (Na₂SO₄) and evaporated to give the title compound as a yellow gum (371 mg, 91%). LCMS RT=2.68 min, ES+ve m/z 453 (M+H)⁺.

### Intermediate 38

### 2-(2-{4-[(6-Butyl-8-quinolinyl)oxy]-1-piperidinyl}ethyl)-1H-isoindole-1,3(2H)-dione

6-Butyl-8-(4-piperidinyloxy)quinoline (for example, as prepared for Intermediate 6) (2.44 g, 8.59 mmol) was stirred with 2-(2-bromoethyl)-1H-isoindole-1,3(2H)-dione (commercially available, for example, from Aldrich) (2.40 g, 9.4 mmol) and potassium carbonate (5.9 g, 43 mmol) in 2-butanone (75 ml) under nitrogen at 80° C. for 3 days. Further 2-(2-bromoethyl)-1H-isoindole-1,3(2H)-dione (2.4 g, 9.4 mmol) was added and the heating and stirring were continued for a further 24 h. More 2-(2-bromoethyl)-1H-isoindole-1,3(2H)-dione (1.2 g, 4.7 mmol) was added and the heating and stirring were continued for a further 24 h. The mixture was cooled and partitioned between water and DCM. The aqueous layer was extracted twice with more DCM (×2) and the combined organic layers were washed with water, dried (MgSO₄) and evaporated to leave an oil. This oil was redissolved in DCM and loaded onto a column of silica gel (250 g) pre-conditioned with DCM. The column was eluted with DCM, then DCM:ethanol:0.880 s.g. aqueous ammonia solution (200:8:1) to give the title compound (2.76 g, 6.03 mmol). LCMS RT=2.91 min, ES+ve m/z 458 [M+H]⁺.

### Intermediate 39

### (2-{4-[(6-Butyl-8-quinolinyl)oxy]-1-piperidinyl}ethyl)amine

2-(2-{4-[(6-Butyl-8-quinolinyl)oxy]-1-piperidinyl}ethyl)-1H-isoindole-1,3(2H)-dione (for example, as prepared for Intermediate 38) (2.76 g, 6.03 mmol) was stirred under nitrogen in ethanol (40 ml) containing hydrazine monohydrate (commercially available, for example, from Aldrich) (0.71 ml, 15.1 mmol) at 80° C. for 2 h. The reaction was cooled with ice-water and filtered. The filter-cake was leached with ethanol and the combined filtrates were evaporated to an oil, containing a white solid. This solid was mixed with DCM (approximately 20 ml) and filtered. The filter-cake was leached with more DCM and the combined filtrates were evaporated to an oil the title compound (2.07 g) LCMS RT=2.32 min, ES+ve m/z 328 [M+H]⁺.

### Intermediate 40

### 1,1-Dimethylethyl (3-{4-[(6-butyl-8-quinolinyl)oxy]-1-piperidinyl}propyl) carbamate

6-Butyl-8-(4-piperidinyloxy)quinoline (for example, as prepared for Intermediate 6) (437 mg, 1.54 mmol) was stirred with 1,1-dimethylethyl (3-bromopropyl)carbamate (commercially available, for example, from Aldrich), (612 mg, 2.57 mmol) and potassium carbonate (426 mg, 3.15 mmol) in 2-butanone (15 ml) under nitrogen at 80° C. overnight. The mixture was cooled and partitioned between water and DCM. The aqueous layer was extracted with more DCM (×2) and the combined organic layers were washed with brine, dried (MgSO₄) and evaporated to give a yellow gum. This was purified by chromatography on silica (50 g, eluting with (1% triethylamine in MeOH)-DCM, 0-30%). The relevant fractions were concentrated in vacuo to give the title compound as a yellow oil (530 mg, 1.20 mmol, 78%). LCMS RT=2.94 min, ES+ve m/z 442 [M+H]⁺.

Intermediates 41 and 42 were prepared in an analogous manner to Intermediate 40:

### Intermediate 41

### 1,1-Dimethylethyl (4-{4-[(6-butyl-8-quinolinyl)oxy]-1-piperidinyl}butyl)carbamate

The title compound was prepared by reacting 6-butyl-8-(4-piperidinyloxy)quinoline (for example, as prepared for Intermediate 6) with 1,1-dimethylethyl (4-bromobutyl)carbamate (commercially available, for example, from Aldrich). LCMS RT=3.04 min, ES+ve m/z 456 [M+H]⁺.

### Intermediate 42

### 1,1-Dimethylethyl (5-{4-[(6-butyl-8-quinolinyl)oxy]-1-piperidinyl}pentyl) carbamate

The title compound was prepared by reacting 6-butyl-8-(4-piperidinyloxy)quinoline (for example, as prepared for Intermediate 6) with 1,1-dimethylethyl (5-bromopentyl)carbamate (commercially available, for example, from Toronto Research Chemicals Inc.). LCMS RT=3.04 min, ES+ve m/z 470 [M+H]⁺.

### Intermediate 43

### (3-{4-[(6-Butyl-8-quinolinyl)oxy]-1-piperidinyl}propyl)amine

1,1-Dimethylethyl (3-{4-[(6-butyl-8-quinolinyl)oxy]-1-piperidinyl}propyl)carbamate (for example, as prepared for Intermediate 40) (493 mg, 1.12 mmol) was treated with a solution of hydrogen chloride in dioxane (4 M, 10 ml), and stirred under nitrogen overnight at room temperature. The mixture was concentrated in vacuo. The residue was dissolved in MeOH, and applied to a SCX-2 ion exchange cartridge (10 g, pre-conditioned with MeOH). The cartridge was washed with MeOH (100 ml), and then eluted with 10% 0.880 s.g. ammonia in MeOH (100 ml). The relevant basic fractions were concentrated in vacuo to give the title compound (292 mg, 0.86 mmol, 76%). LCMS RT=1.92 min, ES+ve m/z 342 (M+H)⁺.

Intermediates 44 and 45 were prepared in an analogous manner to Intermediate 43:

### Intermediate 44

### (4-{4-[(6-Butyl-8-quinolinyl)oxy]-1-piperidinyl}butyl)amine

The title compound was prepared using 1,1-dimethylethyl (4-{4-[(6-butyl-8-quinolinyl)oxy]-1-piperidinyl}butyl)carbamate (for example, as prepared for Intermediate 41). LCMS RT=2.34 min, ES+ve m/z 356 [M+H]⁺.

### Intermediate 45

### (5-{4-[(6-Butyl-8-quinolinyl)oxy]-1-piperidinyl}pentyl)amine

The title compound was prepared using 1,1-dimethylethyl (5-{4-[(6-butyl-8-quinolinyl)oxy]-1-piperidinyl}pentyl)carbamate (for example, as prepared for Intermediate 42). LCMS RT=2.28 min, ES+ve m/z 370 [M+H]⁺.

### Intermediate 46

### 4-(Methyloxy)butanoic Acid

Methyl 4-(methyloxy)butanoate (commercially available, for example, from Aldrich). (8.2 ml, 60 mmol) was dissolved in MeOH (60 ml) and treated with 2 M aqueous sodium hydroxide solution (60 ml), and the resulting mixture was stirred at room temperature overnight. The mixture was concentrated in vacuo to remove the MeOH. The aqueous mixture was partitioned between DCM (100 ml) and water (40 ml). The layers were separated and the aqueous washed with further DCM (100 ml). The aqueous layer was acidified to pH 1-2 using 5 M hydrochloric acid (24 ml), and extracted with DCM (2×100 ml). These latter organic extracts were combined and concentrated in vacuo to give the product as a colourless mobile oil (4.12 g, 34.8 mmol, 58%), ¹H NMR (400 MHz, CDCl₃) δ 11.0, (br s, 1H), 3.44 (t, J=6 Hz, 2H), 3.34 (s, 3H), 2.46 (t, J=7.5 Hz, 2H), 1.95-1.86 (m, 2H).

### Intermediate 47

### Ethyl 4-{4-[(6-pentyl-8-quinolinyl)oxy]-1-piperidinyl}butanoate

6-Pentyl-8-(4-piperidinyloxy)quinoline (for example, as prepared for intermediate 10) (298 mg, 1.0 mmol) was dissolved in DMF (12.5 ml). Potassium carbonate (1.27 g, 5.0 mmol) and ethyl 4-bromobutanoate (commercially available, for example, from Aldrich) (0.20 ml, 1.4 mmol) were added. The mixture was heated to 60° C. with stirring under a nitrogen atmosphere overnight. LCMS analysis showed incomplete reaction. Further potassium carbonate (0.51 g, 2.0 mmol) and ethyl 4-bromobutanoate (0.09 ml, 0.61 mmol) were added and the mixture was again heated to 60° C. with stirring under a nitrogen atmosphere, then left to stand overnight at room temperature. LCMS analysis still showed incomplete reaction. Further potassium carbonate (0.76 g, 3.0 mmol) and ethyl 4-bromobutanoate (0.14 ml, 0.95 mmol) were added and the mixture was again heated to 60° C. with stirring under a nitrogen atmosphere for 20 h. The cooled reaction mixture was filtered, and the filtrate was concentrated in vacuo. The residue was dissolved in ethanol, and this solution was applied to a SCX-2 ion exchange cartridge (50 g, pre-conditioned with MeOH, then ethanol). The cartridge was washed with ethanol (3 column volumes), and then eluted with 10% 0.880 s.g. ammonia in ethanol. The relevant basic fractions were concentrated in vacuo. The residue was further purified by chromatography on silica (50 g, eluting with (1% triethylamine in MeOH)-DCM, 0-15%). The relevant fractions were concentrated in vacuo to give the title compound. LCMS RT=2.93 min, ES+ve m/z 413 (M-FH)⁺

### Intermediate 48

### 4-{4-[(6-Pentyl-8-quinolinyl)oxy]-1-piperidinyl}butanoic acid, partial triethylamine Salt

Ethyl 4-{-4-[(6-pentyl-8-quinolinyl)oxy]-1-piperidinyl}butanoate (for example, as prepared for Intermediate 47) (153 mg, 0.37 mmol) was stirred in a mixture of MeOH (6 ml) and 2 M aqueous sodium hydroxide (5 ml) at room temperature for 30 min. The solvent was removed in vacuo, and the residue was dissolved in water (6 ml) and treated with 2 M aqueous hydrogen chloride (5 ml). This mixture was applied to a SCX-2 ion exchange cartridge (20 g, pre-conditioned with MeOH (1 column volume) and water (2 column volumes)). The cartridge was washed with water (1 column volume), and then eluted with 10% triethylamine in MeOH. The relevant basic fractions were concentrated in vacuo to give the title compound as the partial triethylamine salt, (127 mg, 82%). LCMS RT=2.79 min, ES+ve m/z 385 (M+H)⁺.

### Intermediate 49

### 1,1-Dimethylethyl (3-{4-[(6-pentyl-8-quinolinyl)oxy]-1-piperidinyl}propyl)carbamate

The title compound was prepared in an analogous manner to Intermediate 40, using 6-pentyl-8-(4-piperidinyloxy)quinoline (for example, as prepared for Intermediate 10) and 1,1-dimethylethyl (3-bromopropyl)carbamate (commercially available, for example, from Fluka), but the reaction mixture was only heated for 7 h. LCMS RT=3.16 min, ES+ve m/z 456 [M+H]⁺; ¹H NMR 6 (CD₃OD) 8.74 (1H, dd, J=4, 2 Hz), 8.22 (1H, dd, J=8, 2 Hz), 7.49 (1H, dd, J=8, 4 Hz), 7.28 (1H, s), 7.12 (1H, d, J=1.5 Hz), 4.82-4.75 (1H, m, obscured by water), 3.20-3.08 (4H, m), 2.78 (2H, t, J=7.5 Hz), 2.75-2.62 (4H, m), 2.20-2.13 (2H, m), 2.13-2.03 (2H, m), 1.82-1.69 (4H, m), 1.43 (9H, s), 1.41-1.34 (4H, m), 0.91 (3H, t, J=7 Hz).

### Intermediate 50

### (3-{4-[(6-Pentyl-8-quinolinyl)oxy]-1-piperidinyl}propyl)amine

The title compound was prepared in an analogous manner to Intermediate 43, using 1,1-dimethylethyl (3-{4-[(6-pentyl-8-quinolinyl)oxy]-1-piperidinyl}propyl)carbamate (for example, as prepared for Intermediate 49). LCMS RT=2.56 min, ES+ve m/z 356 [M+H]⁺; ¹H NMR 6 (CD₃OD) 8.72 (1H, dd, J=4, 2 Hz), 8.20 (1H, dd, J=8, 2 Hz), 7.47 (1H, dd, J=8, 4 Hz), 7.25 (1H, s), 7.09 (1H, d, J=1.5 Hz), 4.72-4.63 (1H, m), 3.01-2.93 (2H, m), 2.83-2.74 (4H, m), 2.51 (2H, t, J=7 Hz), 2.44-2.36 (2H, m), 2.19-2.10 (2H, m), 2.04-1.92 (2H, m), 1.81-1.67 (4H, m), 1.42-1.32 (4H, m), 0.90 (3H, t, J=7 Hz).

### EXAMPLES

### Example 1

### 8-({1-[(1-Acetyl-4-piperidinyl)methyl]-4-piperidinyl}oxy)-6-butylquinoline, hydrochloride salt

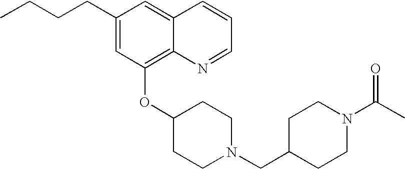

Acetyl chloride (commercially available, for example, from Aldrich) (10.6 μl, 0.15 mmol) and triethylamine (41.4 μl, 0.3 mmol) were added to a solution of 6-butyl-8-{[1-(4-piperidinylmethyl)-4-piperidinyl]oxy}quinoline (for example, as prepared for Intermediate 16) (38 mg, 0.1 mmol) in DCM (0.5 ml). The mixture was stirred at room temperature over the weekend. The mixture was filtered and the filtrate was loaded onto an SCX ion-exchange cartridge (1 g, pre-conditioned with MeOH). The cartridge was eluted with MeOH (2 column volumes) followed by 2 N ammonia in MeOH (3 column volumes). Evaporation of the solvent from the ammoniacal eluent gave a residue that was dissolved in 1.25 M hydrogen chloride in MeOH (3 ml). The solvent was then evaporated giving the title compound as a yellow solid (46.8 mg). LCMS RT=2.33 min, ES+ve m/z 424 [M+H]⁺.

### Example 2

### 6-Butyl-8-({1-[(1-propanoyl-4-piperidinyl)methyl]-4-piperidinyl}oxy)quinoline, hydrochloride salt

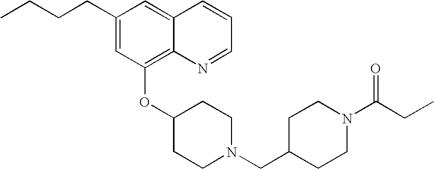

Propionyl chloride (commercially available, for example, from Aldrich) (13 μl, 0.15 mmol) and triethylamine (41.4 μl, 0.3 mmol) were added to a solution of 6-butyl-8-{[1-(4-piperidinylmethyl)-4-piperidinyl]oxy}quinoline (for example, as prepared for Intermediate 16) (38 mg, 0.1 mmol) in DCM (0.5 ml). The mixture was stirred at room temperature over the weekend. The mixture was filtered and the filtrate was loaded onto an SCX ion-exchange cartridge (1 g, pre-conditioned with DCM). The cartridge was eluted with DCM (2 column volumes) followed by 2 N ammonia in MeOH (3 column volumes). Evaporation of the solvent from the ammoniacal eluent gave a residue that was dissolved in 1.25 M hydrogen chloride in MeOH (3 ml). The solvent was then evaporated giving the title compound as a yellow solid (47.3 mg). LCMS RT=2.39 min, ES+ve m/z 438 [M+H]⁺.

### Example 3

### 8-({1-[2-(1-Acetyl-4-piperidinyl)ethyl]-4-piperidinyl}oxy)-6-butylquinoline, hydrochloride salt

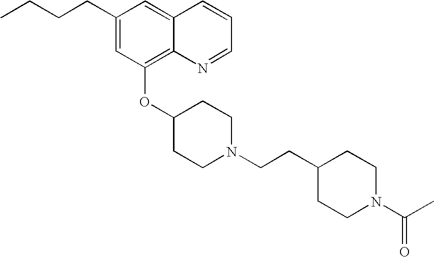

Acetyl chloride (commercially available, for example, from Aldrich) (10.24 μl, 0.14 mmol) and triethylamine (39.9 μl, 0.29 mmol) were added to a solution of 6-butyl-8-({1-[2-(4-piperidinyl)ethyl]-4-piperidinyl}oxy)quinoline (for example, as prepared for Intermediate 18) (38 mg, 0.096 mmol) in DCM (0.5 ml). The mixture was stirred at room temperature over the weekend. The mixture was filtered and the filtrate was loaded onto an conditioned SCX ion-exchange cartridge (1 g. pre-conditioned with MeOH). The cartridge was eluted with MeOH (2 column volumes) followed by 2 N ammonia in MeOH (3 column volumes). Evaporation of the solvent from the ammoniacal eluent gave a residue that was further purified by MDAP HPLC. Evaporation of the solvent from the appropriate fractions gave a residue that was dissolved in 1.25 M hydrogen chloride in MeOH (3 ml). The solvent was then evaporated giving the title compound as a yellow solid (26.9 mg). LCMS RT=2.37 min, ES+ve m/z 438 [M+H]⁺.

### Example 4

### 6-Butyl-8-({1-[2-(1-propanoyl-4-piperidinyl)ethyl]-4-piperidinyl}oxy)quinoline, hydrochloride salt

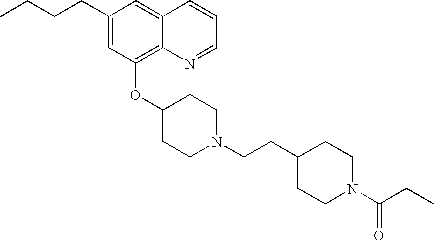

Propionyl chloride (commercially available, for example, from Aldrich) (12.52 μl, 0.14 mmol) and triethylamine (39.9 μl, 0.29 mmol) were added to a solution of 6-butyl-8-({1-[2-(4-piperidinyl)ethyl]-4-piperidinyl}oxy)quinoline (for example, as prepared for Intermediate 18) (38 mg, 0.096 mmol) in DCM (0.5 ml). The mixture was stirred at room temperature over the weekend. The mixture was filtered and the filtrate was loaded onto an SCX ion-exchange cartridge (1 g, pre-conditioned with MeOH). The cartridge was eluted with MeOH (2 column volumes) followed by 2 N ammonia in MeOH (3 column volumes). Evaporation of the solvent from the ammoniacal eluent gave a residue that was further purified by MDAP HPLC. Evaporation of the solvent from the appropriate fractions gave a residue that was dissolved in 1.25 M hydrogen chloride in MeOH (3 ml). The solvent was then evaporated giving the title compound as a yellow solid (22.3 mg). LCMS RT=2.44 min, ES+ve m/z 452 [M+H]⁺.

### Example 5

### 3-({4-[(6-butyl-8-quinolinyl)oxy]-1-piperidinyl}methyl)-2-piperidinone, formate salt (1:1)

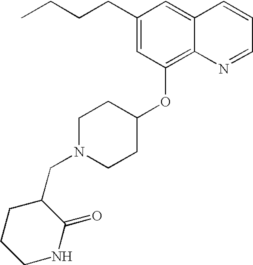

(2-Oxo-3-piperidinyl)methyl methanesulfonate (for example, as prepared for Intermediate 19) (45 mg, 0.22 mmol) was heated with 6-butyl-8-(4-piperidinyloxy)quinoline (for example, as prepared for Intermediate 6) (48 mg, 0.17 mmol) sodium bicarbonate (114 mg, 1.36 mmol) plus sodium iodide (26 mg, 0.17 mmol) in DMF (1 ml) in a microwave oven with stirring at 150° C. for 15 min. The reaction was incomplete. The above microwave heating was repeated for 20 min and then 30 min. The DMF was evaporated and the residue in DCM was loaded onto a bond elute silica cartridge (10 g, pre-conditioned with DCM). The cartridge was eluted with DCM, then DCM: ethanol: 0.880 s.g. aqueous ammonia solution in water (200:8:1, then 100:8:1). Evaporation of the appropriate fractions gave impure title compound (12 mg), LCMS RT=2.55 min, ES+ve m/z 396 (M+H)⁺. This was purified by MDAP HPLC using an extended run to give the title compound (6.95 mg), LCMS RT=2.51 min, ES+ve m/z 396 (M+H)⁺.

### Example 6

### 4-{4-[(6-Butyl-8-quinolinyl)oxy]-1-piperidinyl}-N-(1,1-dimethylethyl)butanamide, dihydrochloride salt

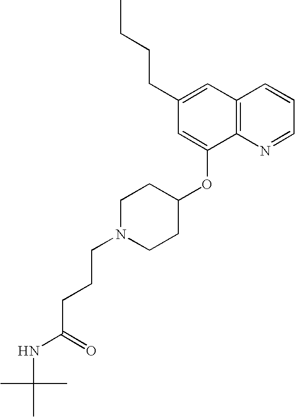

4-{4-[(6-Butyl-8-quinolinyl)oxy]-1-piperidinyl}butanoic acid, partial triethylamine salt (for example, as prepared for Intermediate 28) (65 mg, 0.14 mmol) was dissolved in DMF (1 ml), and triethylamine (57 μl, 0.41 mmol) and TBTU (66 mg, 0.21 mmol) were added. This mixture was stirred at room temperature for 10 min, and then t-butylamine (commercially available, for example, from Aldrich) (29 μl, 0.28 mmol) and further DMF (1 ml) were added. The mixture was stirred at room temperature for 90 min, then left to stand at room temperature overnight. The mixture was applied to an SCX-2 ion exchange cartridge (20 g, pre-conditioned with MeOH). The cartridge was washed with MeOH (100 ml), and then eluted with 10% 0.880 s.g. ammonia in MeOH (100 ml). Relevant basic fractions were combined and concentrated. The residue was purified by MDAP HPLC. The appropriate fractions were combined, and the solvent was removed. The residue was dissolved in MeOH, and the solution was treated with a solution of hydrogen chloride in MeOH (1.25 M, 0.5 ml, 0.6 mmol). The solvent was removed by evaporation under a stream of nitrogen to give the title compound as a pale yellow glass (52 mg, 76%). LCMS RT=2.85 min, ES+ve m/z 426 (M+H)⁺.

### Example 7

### 3-{4-[(6-Butyl-8-quinolinyl)oxy]-1-piperidinyl}-N,N-diethylpropanamide, dihydrochloride Salt

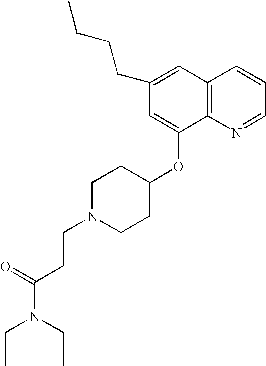

The title compound was prepared in an analogous manner to Example 6, using 3-{4-[(6-butyl-8-quinolinyl)oxy]-1-piperidinyl}propanoic acid, triethylamine salt (for example, as prepared for Intermediate 30) and diethylamine (commercially available, for example, from Aldrich). LCMS RT=2.72 min, ES+ve m/z 412 (M+H)⁺.

### Examples 8 to 32

General method for the preparation of Examples 8 to 32:

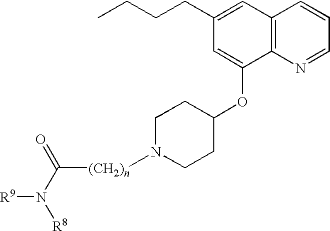

It will be appreciated that (CH₂)ₙ above corresponds to the straight chain C₁₋₆alkylene of R⁷.

The acid as its triethylamine salt (20 mg, 0.41-0.045 mmol), PyBOP (23 mg, 0.045 mmol) and N,N′-diisopropylethylamine (16 μl, 0.090 mmol) were combined in DMF (0.2 ml). The amine (20 μl, excess) was added to the reaction solution, which was shaken, then left to stand at room temperature for 18 h. HATU (17 mg, 0.045 mmol), N,N′-diisopropylethylamine (20 μl, 0.12 mmol) and further amine (20 μl, excess) were added to the reaction solution, which was shaken and again left to stand at room temperature for 18 h. Further HATU (17 mg, 0.045 mmol) N,N′-diisopropylethylamine (20 μl, 0.12 mmol) and amine (20 μl, excess) were added and the mixture was left for 72 h. The mixture was applied to an aminopropyl SPE cartridge (0.5 g, pre-conditioned with chloroform). The cartridge was washed with chloroform, followed by a solution of 10% MeOH in EtOAc to elute the amide. Relevant fractions were combined and evaporated to dryness under a stream of nitrogen. The residue was purified on a high pH column using MDAP HPLC. The appropriate fractions were combined, and the solvent was removed in vacuo. The residue was dissolved in a solution of hydrogen chloride in MeOH (1.25 M, 0.5 ml). The solvent was removed by evaporation under a stream of nitrogen to give the amide as the dihydrochloride salt.

If n=1, the acid used was 2-{4-[(6-butyl-8-quinolinyl)oxy]-1-piperidinyl}acetic acid, triethylamine salt (for example, as prepared for Intermediate 29).

If n=2, the acid used was 3-{4-[(6-butyl-8-quinolinyl)oxy]-1-piperidinyl}propanoic acid, triethylamine salt (for example, as prepared for Intermediate 30).

If n=3, the acid used was 4-{4-[(6-butyl-8-quinolinyl)oxy]-1-piperidinyl}butanoic acid, triethylamine salt (for example, as prepared for Intermediate 28).

If n=4, the acid used was 5-{4-[(6-butyl-8-quinolinyl)oxy]-1-piperidinyl}pentanoic acid, triethylamine salt (for example, as prepared for Intermediate 31).

The following amines were used:

Ethylamine, 3-methoxypropylamine, diethylamine, propylamine, 2-methoxyethylamine, tert-butylamine and piperidine (all of which are commercially available, for example, from Aldrich).

| | | | | | LCMS | |
|---|---|---|---|---|---|---|
| Example | | | | Amine | RT | ES + ve |
| No. | Compound Name | n = | Structure | (NR⁸R⁹) | (min) | m/z |
| 8 | 2-{4-[(6-butyl-8- quinolinyl)oxy]-1- piperidinyl}-N-[3- (methyloxy)propyl] acetamide | 1 | 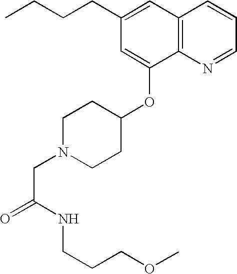 | 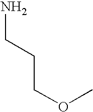 | 2.64 | 414 |
| 9 | 2-{4-[(6-butyl-8- quinolinyl)oxy]-1- piperidinyl}-N,N- diethylacetamide | 1 | 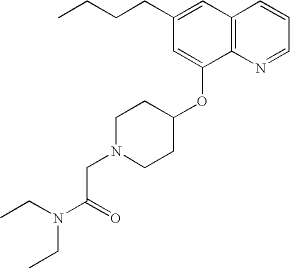 | 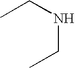 | 2.39 | 398 |
| 10 | 2-{4-[(6-butyl-8- quinolinyl)oxy]-1- piperidinyl}-N- propylacetamide | 1 | 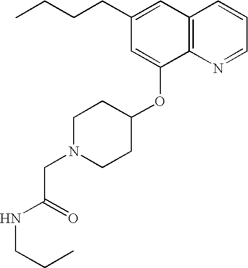 | 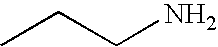 | 2.38 | 384 |
| 11 | 2-{4-[(6-butyl-8- quinolinyl)oxy]-1- piperidinyl}-N-[2- (methyloxy)ethyl] acetamide | 1 | 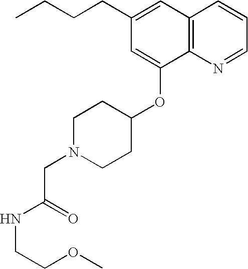 | 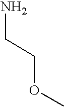 | 2.26 | 400 |
| 12 | 2-{4-[(6-butyl-8- quinolinyl)oxy]-1- piperidinyl}-N-(1,1- dimethylethyl) acetamide | 1 | 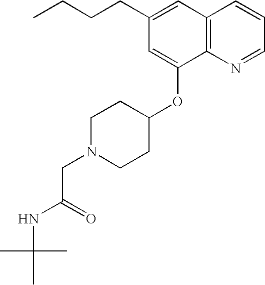 | 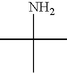 | 2.47 | 398 |
| 13 | 6-butyl-8-({1-[2-oxo-2- (1-piperidinyl)ethyl]-4- piperidinyl}oxy) quinoline | 1 | 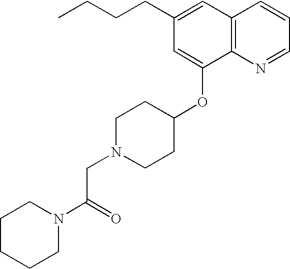 | 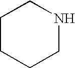 | 2.39 | 410 |
| 14 | 2-{4-[(6-butyl-8- quinolinyl)oxy]-1- piperidinyl}-N-(1- methylethyl)acetamide | 1 | 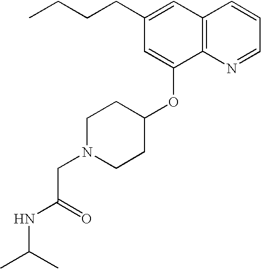 | 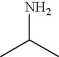 | 2.33 | 384 |
| 15 | 3-{4-[(6-butyl-8- quinolinyl)oxy]-1- piperidinyl}-N- ethylpropanamide | 2 | 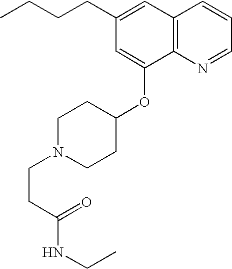 | 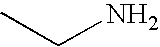 | 2.56 | 384 |
| 16 | 3-{4-[(6-butyl-8- quinolinyl)oxy]-1- piperidinyl}-N-[3- (methyloxy)propyl] propanamide | 2 | 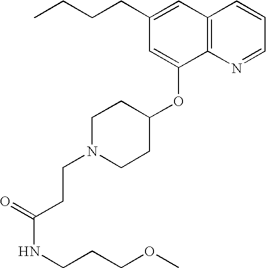 | 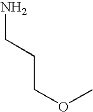 | 2.58 | 428 |
| 17 | 3-{4-[(6-butyl-8- quinolinyl)oxy]-1- piperidinyl}-N-(1,1- dimethylethyl) propanamide | 2 | 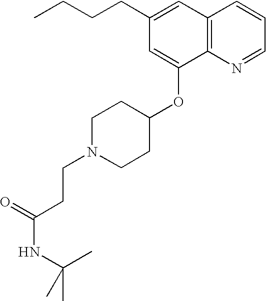 | 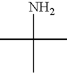 | 2.83 | 412 |
| 18 | 6-butyl-8-({1-[3-oxo-3- (1-piperidinyl)propyl]-4- piperidinyl}oxy) quinoline | 2 | 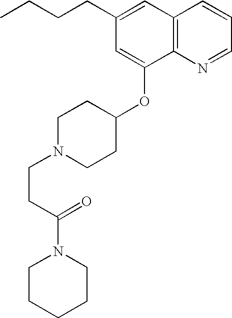 | 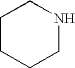 | 2.76 | 424 |
| 19 | 3-{4-[(6-butyl-8- quinolinyl)oxy]-1- piperidinyl}-N-(1- methylethyl) propanamide | 2 | 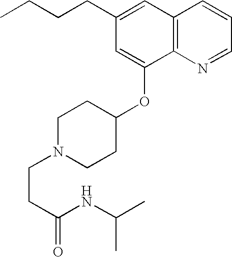 | 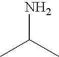 | 2.63 | 398 |
| 20 | 4-{4-[(6-butyl-8- quinolinyl)oxy]-1- piperidinyl}-N- ethylbutanamide | 3 | 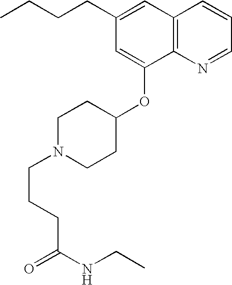 | 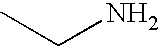 | 2.61 | 398 |
| 21 | 4-{4-[(6-butyl-8- quinolinyl)oxy]-1- piperidinyl}-N-[3- (methyloxy)propyl] butanamide | 3 | 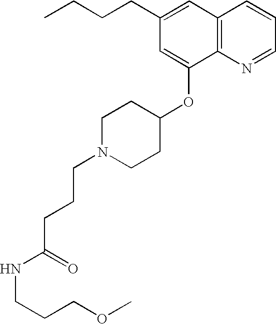 | 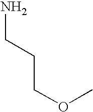 | 2.6 | 442 |
| 22 | 4-{4-[(6-butyl-8- quinolinyl)oxy]-1- piperidinyl}-N- propylbutanamide | 3 | 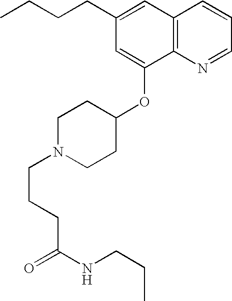 | 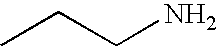 | 2.71 | 412 |
| 23 | 4-{4-[(6-butyl-8- quinolinyl)oxy]-1- piperidinyl}-N-[2- (methyloxy)ethyl] butanamide | 3 | 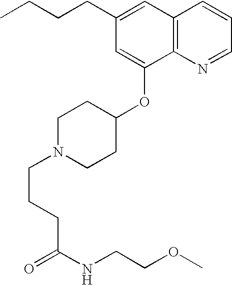 | 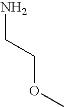 | 2.54 | 428 |
| 24 | 6-butyl-8-({1-[4-oxo-4- (1-piperidinyl)butyl]-4- piperidinyl}oxy)quinoline | 3 | 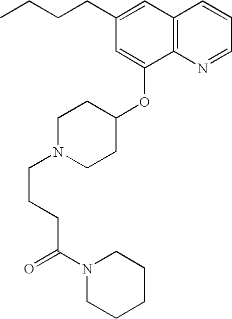 | 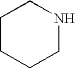 | 2.79 | 438 |
| 25 | 4-{4-[(6-butyl-8- quinolinyl)oxy]-1- piperidinyl}-N-(1- methylethyl)butanamide | 3 | 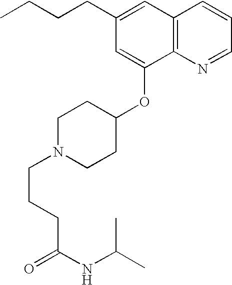 | 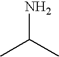 | 2.64 | 412 |
| 26 | 5-{4-[(6-butyl-8- quinolinyl)oxy]-1- piperidinyl}-N-[3- (methyloxy)propyl]pent- anamide | 4 | 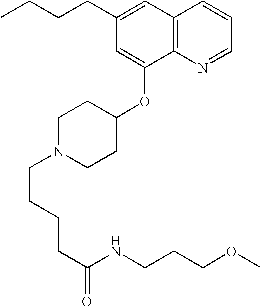 | 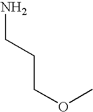 | 2.6 | 456 |
| 27 | 5-{4-[(6-butyl-8- quinolinyl)oxy]-1- piperidinyl}-N,N- diethylpentanamide | 4 | 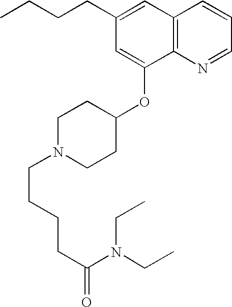 | 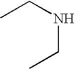 | 2.79 | 440 |
| 28 | 5-{4-[(6-butyl-8- quinolinyl)oxy]-1- piperidinyl}-N- propylpentanamide | 4 | 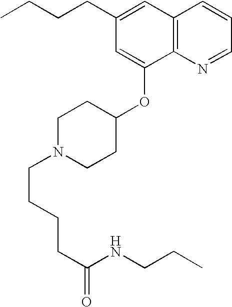 | 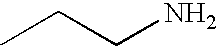 | 2.72 | 426 |
| 29 | 5-{4-[(6-butyl-8- quinolinyl)oxy]-1- piperidinyl}-N-[2- (methyloxy)ethyl] pentanamide | 4 | 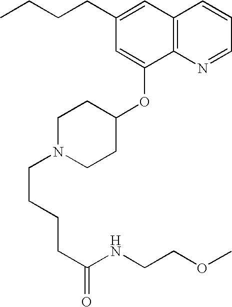 | 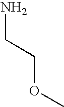 | 2.56 | 442 |
| 30 | 5-{4-[(6-butyl-8- quinolinyl)oxy]-1- piperidinyl}-N-(1,1- dimethylethyl) pentanamide | 4 | 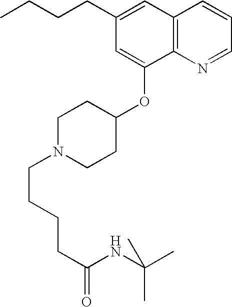 | 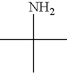 | 2.85 | 440 |
| 31 | 6-butyl-8-({1-[5-oxo-5- (1-piperidinyl)pentyl]-4- piperidinyl}oxy) quinoline | 4 | 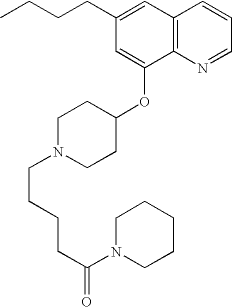 | 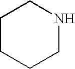 | 2.81 | 452 |
| 32 | 5-{4-[(6-butyl-8- quinolinyl)oxy]-1- piperidinyl}-N-(1- methylethyl) pentanamide | 4 | 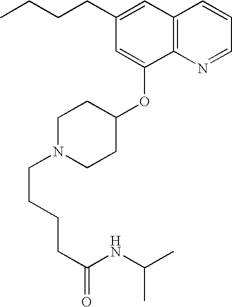 | 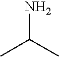 | 2.65 | 426 |

### Example 33

### 6-butyl-8-({(3R)-1-[3-oxo-3-(1-piperidinyl)propyl]-3-pyrrolidinyl}oxy)quinoline, formate salt (1:1)

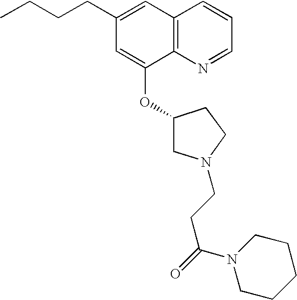

To a solution of 3-{(3R)-3-[(6-butyl-8-quinolinyl)oxy]-1-pyrrolidinyl}propanoic acid (for example, as prepared for Intermediate 32) (0.050 g, 0.146 mmol) in DMF (3 ml) was added TBTU (0.050 g, 0.156 mmol) and then triethylamine (0.1 ml, 0.72 mmol). The solution was allowed to stand at ambient temperature for 5 min. Piperidine (commercially available, for example, from Aldrich) (0.018 g, 0.23 mmol) was added to the solution, and stirred at ambient temperature overnight. The mixture was applied to an SCX-2 cartridge (20 g, pre-conditioned with MeOH) and the cartridge was washed with MeOH (2 column volumes). The cartridge was eluted with 10% 0.880 s.g. ammonia in MeOH (2 column volumes). The basic fractions were concentrated in vacuo to leave a yellow gum. The residue was purified by MDAP HPLC and the appropriate fractions combined. The solvent was removed in vacuo to give the title compound as a formate salt (0.019 g, 28%). LCMS RT=2.83 min, ES+ve m/z 410 (M+H)⁺.

### Example 34

### 6-butyl-8-({(3R)-1-[4-oxo-4-(1-piperidinyl)butyl]-3-pyrrolidinyl}oxy)quinoline, formate salt (1:1)

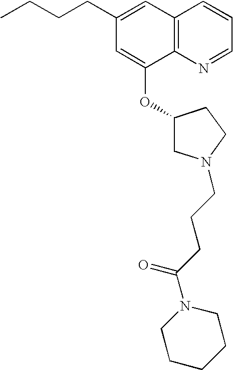

The title compound was prepared in an analogous manner to Example 33 using 4-{(3R)-3-[(6-butyl-8-quinolinyl)oxy]-1-pyrrolidinyl}butanoic acid (for example, as prepared for Intermediate 33). LCMS RT=3.07 min, ES+ve m/z 424 (M+H)⁺.

### Examples 35 to 37

Examples 35 to 37 were prepared in an analogous manner to Example 33

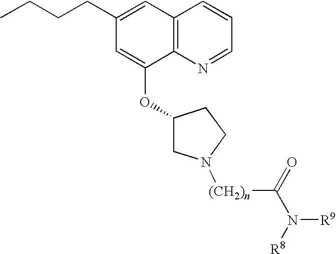

It will be appreciated that (CH₂)ₙ above corresponds to the straight chain C₁₋₆alkylene of R⁷.

If n=2, the acid used was 3-{(3R)-3-[(6-butyl-8-quinolinyl)oxy]-1-pyrrolidinyl}propanoic acid (for example, as prepared for Intermediate 32).

If n=3, the acid used was 3-{(3R)-3-[(6-butyl-8-quinolinyl)oxy]-1-pyrrolidinyl}butanoic acid (for example, as prepared for Intermediate 33).

The following amines were used:

Piperidine, tert-butylamine, diethylamine, piperidine (all of which are commercially available, for example, from Aldrich).

| | | | | | LCMS | |
|---|---|---|---|---|---|---|
| Example | | | | Amine | RT | ES + ve |
| No | Compound Name | n = | Structure | (NR⁸R⁹) | (min) | m/z |
| 35 | 3-{(3R)-3-[(6-butyl-8- quinolinyl)oxy]-1- pyrrolidinyl}-N,N- diethylpropanamide | 2 | 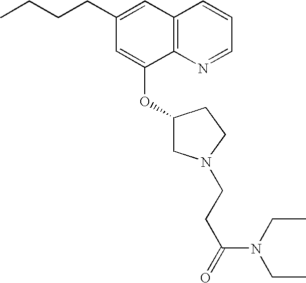 | 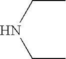 | 2.81 | 398 |
| 36 | 3-{(3R)-3-[(6-butyl-8- quinolinyl)oxy]-1- pyrrolidinyl}-N-(1,1- dimethylethyl) propanamide | 2 | 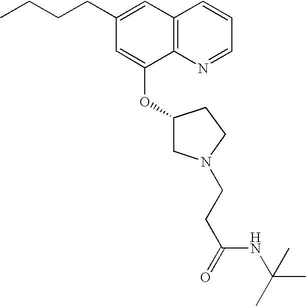 | 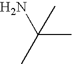 | 2.92 | 398 |
| 37 | 4-{(3R)-3-[(6-butyl-8- quinolinyl)oxy]-1- pyrrolidinyl}-N-(1,1- dimethylethyl) butanamide | 3 | 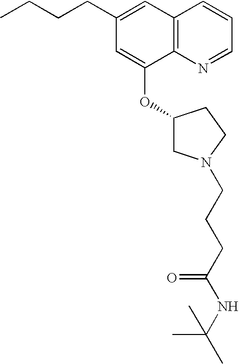 | 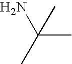 | 3.08 | 412 |

### Example 38

### 6-butyl-8-({(3S)-1-[3-oxo-3-(1-piperidinyl)propyl]-3-pyrrolidinyl}oxy)quinoline, formate salt (1:1)

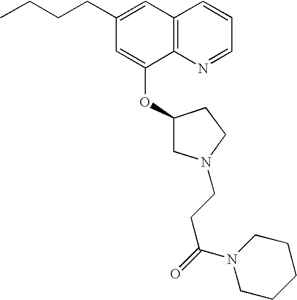

The title compound was prepared in an analogous manner to Example 33, using 3-{(3S)-3-[(6-butyl-8-quinolinyl)oxy]-1-pyrrolidinyl}propanoic acid (for example, as prepared for Intermediate 34). LCMS RT=2.68 min, ES+ve m/z 410 (M+H)⁺.

### Example 39

### 6-butyl-8-({(3S)-1-[4-oxo-4-(1-piperidinyl)butyl]-3-pyrrolidinyl}oxy)quinoline, formate salt (1:1)

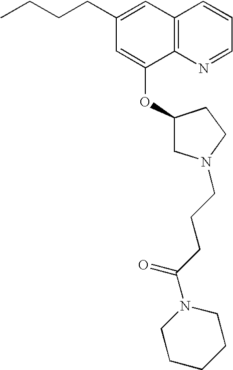

The title compound was prepared in an analogous manner to Example 33, using 3-{(3S)-3-[(6-butyl-8-quinolinyl)oxy]-1-pyrrolidinyl}butanoic acid (for example, as prepared for Intermediate 35). LCMS RT=3.05 min, ES+ve m/z 424 (M+H)⁺.

### Examples 40 and 41

Examples 40 and 41 were prepared in an analogous manner to for Example 33.

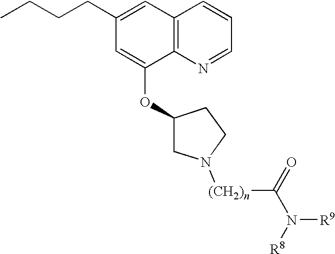

It will be appreciated that (CH₂)ₙ above corresponds to the straight chain C₁₋₆alkylene of R⁷.

When n=2, the acid used was 3-{(3S)-3-[(6-butyl-8-quinolinyl)oxy]-1-pyrrolidinyl}propanoic acid (for example, as prepared for Intermediate 34).

When n=3, the acid used was 3-{(3S)-3-[(6-butyl-8-quinolinyl)oxy]-1-pyrrolidinyl}butanoic acid (for example, as prepared for Intermediate 35).

The following amines were used:

Diethylamine and tert-butylamine (both of which are commercially available, for example, from Aldrich).

| | | | | | LCMS | |
|---|---|---|---|---|---|---|
| Example | | | | Amine | RT | ES + ve |
| No. | Compound Name | n = | Structure | (NR⁸R⁹) | (min) | m/z |
| 40 | 3-{(3S)-3-[(6-butyl-8- quinolinyl)oxy]-1- pyrrolidinyl}-N,N- diethylpropanamide | 2 | 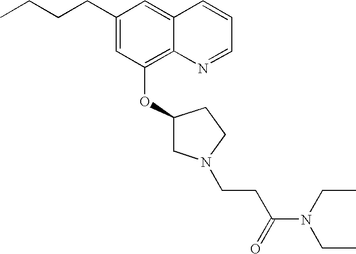 | 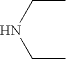 | 2.67 | 398 |
| 41 | 4-{(3S)-3-[(6-butyl-8- quinolinyl)oxy]-1- pyrrolidinyl}-N-(1,1- dimethylethyl) butanamide | 3 | 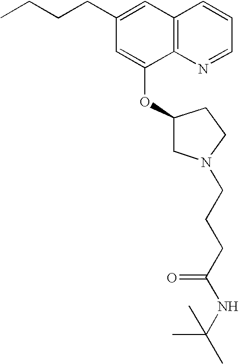 | 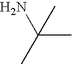 | 3.06 | 412 |

### Example 42

### Trans-4-({4-[(6-butyl-8-quinolinyl)oxy]-1-piperidinyl}methyl)-N-(1-methylethyl)cyclohexanecarboxamide, dihydrochloride salt

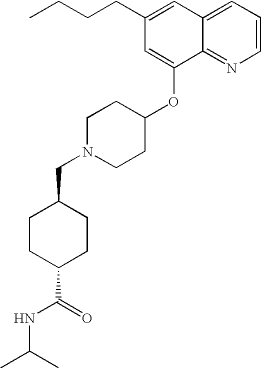

To a solution of trans-4-({4-[(6-butyl-8-quinolinyl)oxy]-1-piperidinyl}methyl)cyclohexanecarboxylic acid (for example, as prepared for Intermediate 36) (0.065 g, 0.153 mmol) in anhydrous DMF (2.0 ml) was added triethylamine (0.1 ml) and then TBTU (0.08 g, 0.25 mmol). The solution was stirred at room temperature for 5 min. To the solution was added isopropylamine (commercially available, for example, from Aldrich) (0.034 ml, 0.023 g). The solution was stirred at ambient temperature for 4 h. The mixture was applied to a SCX ion exchange cartridge (10 g, pre-conditioned with MeOH) and the cartridge washed with MeOH (2 column volumes). The cartridge was eluted with 10% 0.880 s.g. ammonia in MeOH (2 column volumes) and the basic fractions concentrated in vacuo to leave a yellow gum. The residue was purified by MDAP HPLC and the appropriate fractions combined. The solvent was removed in vacuo to leave a colourless gum (0.0345 g). The residue was dissolved in MeOH and to the solution was added 1.25 M hydrogen chloride in MeOH (0.5 ml). The solvent was removed under a stream of nitrogen to give the title compound as a yellow solid (0.032 g, 39%). LCMS RT=2.80 min, ES+ve m/z 466 (M+H)⁺.

### Examples 43 to 48

Examples 43 to 48 were prepared in an analogous manner to Example 42:

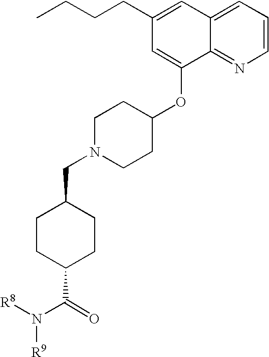

The following amines were used:

Isopropylamine, diethylamine, piperidine, propylamine, neopentylamine and 3-methoxypropylamine (all of which are commercially available for example from Aldrich or TCI-Europe).

| | | | | LCMS | |
|---|---|---|---|---|---|
| Example | | | Amine | RT | ES + ve |
| No. | Compound Name | Structure | (NR⁸R⁹) | (min) | m/z |
| 43 | Trans-4-({4-[(6-butyl- 8-quinolinyl)oxy]-1- piperidinyl} methyl)-N,N- diethylcyclohexane carboxamide | 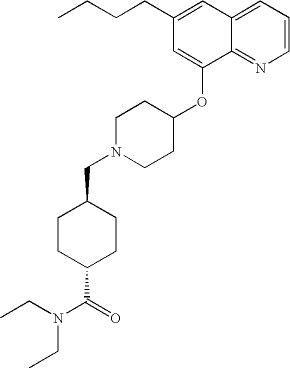 | 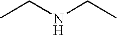 | 2.89 | 480 |
| 44 | 6-Butyl-8-[(1-{[trans-4- (1-piperidinyl carbonyl)cyclohexyl] methyl}-4-piperidinyl) oxy]quinoline | 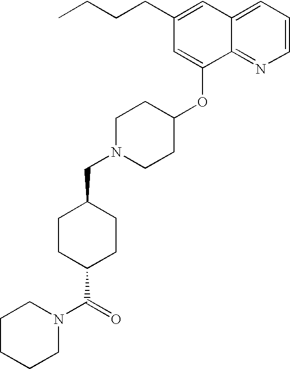 | 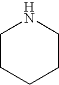 | 3.94 | 492 |
| 45 | Trans-4-({4-[(6-butyl- 8-quinolinyl)oxy]-1- piperidinyl}methyl)-N- propylcyclohexane carboxamide | 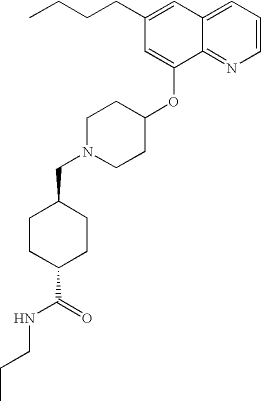 | 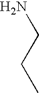 | 2.82 | 466 |
| 46 | trans-4-({4-[(6-butyl-8- quinolinyl)oxy]-1- piperidinyl}methyl)-N- (2,2-dimethylpropyl) cyclohexane carboxamide | 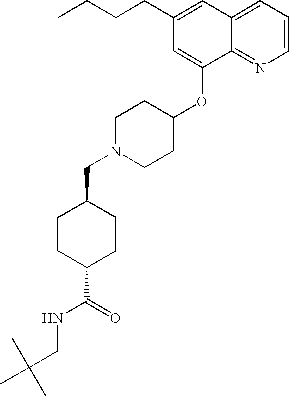 | 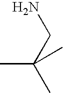 | 3.05 | 494 |
| 47 | trans-4-({4-[(6-butyl-8- quinolinyl)oxy]-1- piperidinyl}methyl)-N- [3-(methyloxy) propyl]cyclohexane carboxamide | 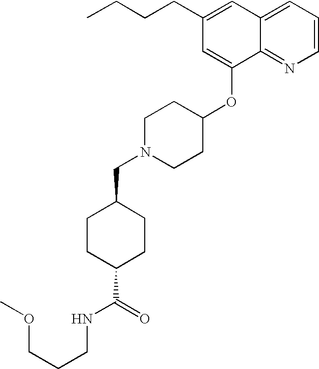 | 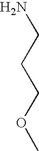 | 2.73 | 496 |

### Examples 48 to 63

General method for the preparation of Examples 48 to 63:

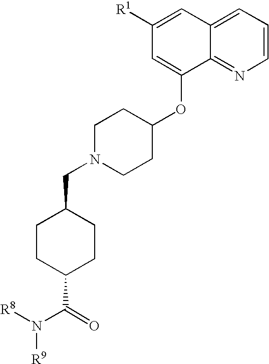

4-({4-[(6-Hexyl-8-quinolinyl)oxy]-1-piperidinyl}methyl)cyclohexanecarboxylic acid (for example, as prepared for Intermediate 37), (362 mg, 0.8 mmol) or 4-({4-[(6-butyl-8-quinolinyl)oxy]-1-piperidinyl}methyl)cyclohexanecarboxylic acid (for example, as prepared for Intermediate 36, (340 mg, 0.8 mmol) in DMF (2 ml) was treated with TBTU (304 mg, 0.8 mmol) and N,NL-diisopropylethylamine (0.25 ml, 1.43 mmol). After standing for 10 min, an aliquot of this solution (0.2 ml, 0.066 mmol) was dispensed into the amine (0.1 mmol) shown in the table below [for example benzylamine (12.1 mg, 0.1 mmol)]. After 16 h, the mixture was concentrated and purified by MDAP HPLC (method B). The appropriate fractions were concentrated in vacuo, and then were further purified by MDAP HPLC (method C) to give the title compound as the free base. This was re-dissolved in MeOH and treated with excess hydrogen chloride in MeOH, the solvent was removed in vacuo to give the title compound as hydrochloride salt (5.3 mg). LCMS RT=3.02 min, ES+ve m/z 556

The following amines were used:

4-methyl aniline, 4-methylbenzylamine, 3-methyl butylamine, piperidine, 2,2-dimethylpropyl amine, diethylamine, pyrrolidine, 1-aminopropane, isopropylamine, morpholine, 3-methoxypropylamine (all of which are commercially available, for example, from Aldrich).

| | | | | | LCMS | |
|---|---|---|---|---|---|---|
| Example | | | | Amine | RT | ES + ve |
| No. | Compound Name | R¹ | Product | (NR⁸R⁹) | (min) | m/z |
| 48 | trans-4-({4-[(6-hexyl-8- quinolinyl)oxy]-1- piperidinyl}methyl)-N- (4-methylphenyl) cyclohexane carboxamide | hexyl | 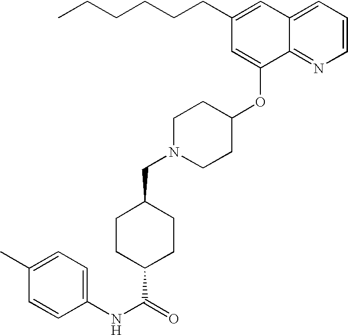 | 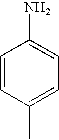 | 3.06 | 542 |
| 49 | trans-4-({4-[(6-hexyl-8- quinolinyl)oxy]-1- piperidinyl}methyl)-N- [(4-methylphenyl) methyl]cyclohexane carboxamide | hexyl | 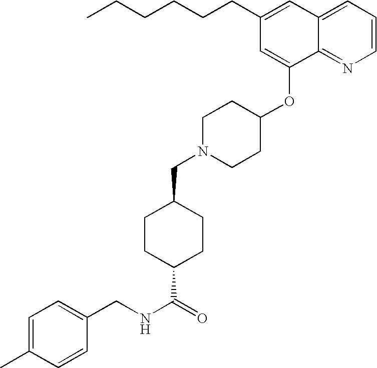 | 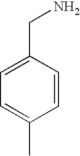 | 3.02 | 556 |
| 50 | trans-4-({4-[(6-hexyl-8- quinolinyl)oxy]-1- piperidinyl}methyl)-N- (3-methylbutyl) cyclohexane carboxamide | hexyl | 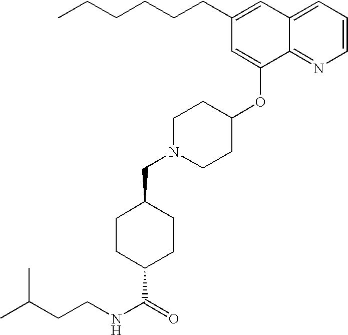 | 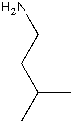 | 2.96 | 522 |
| 51 | 6-hexyl-8-[(1-{[trans-4- (1-piperidinyl carbonyl)cyclohexyl] methyl}-4-piperidinyl) oxy]quinoline | hexyl | 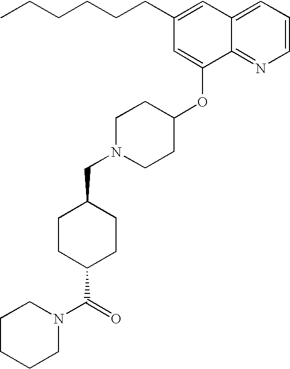 | 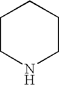 | 2.86 | 520 |
| 52 | trans-N-(2,2- dimethylpropyl)-4-({4- [(6-hexyl-8-quinolinyl) oxy]-1-piperidinyl} methyl)cyclohexane carboxamide | hexyl | 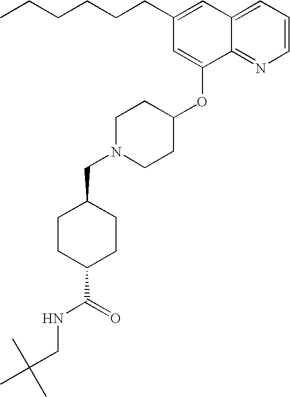 | 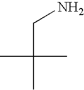 | 2.94 | 522 |
| 53 | trans-N,N-diethyl-4-({4- [(6-hexyl-8- quinolinyl)oxy]-1- piperidinyl}methyl) cyclohexane carboxamide | hexyl | 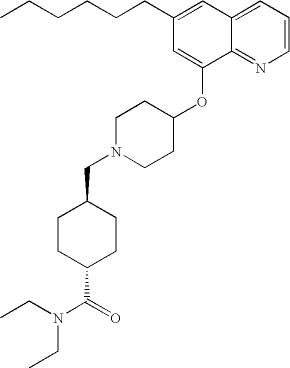 | 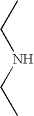 | 2.83 | 508 |
| 54 | 6-hexyl-8-[(1-{[trans-4- (1-pyrrolidinyl carbonyl)cyclohexyl] methyl}-4-piperidinyl) oxy]quinoline | hexyl | 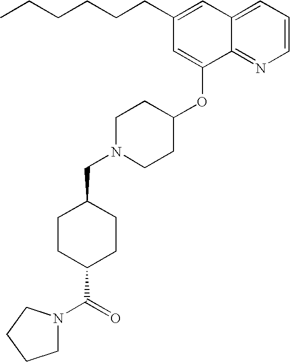 | 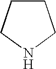 | 2.77 | 506 |
| 55 | trans-4-({4-[(6-hexyl-8- quinolinyl)oxy]-1- piperidinyl}methyl)-N- propylcyclohexane carboxamide | hexyl | 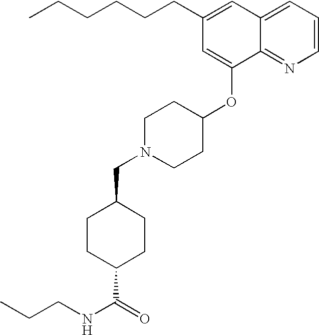 | 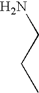 | 2.79 | 494 |
| 56 | trans-4-({4-[(6-hexyl-8- quinolinyl)oxy]-1- piperidinyl}methyl)-N- (1-methylethyl) cyclohexane carboxamide | hexyl | 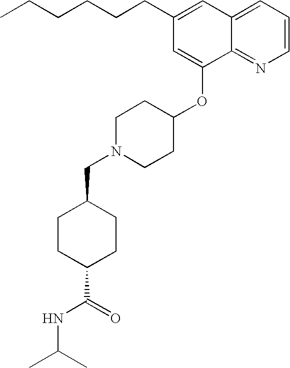 | 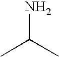 | 2.77 | 494 |
| 57 | 6-hexyl-8-[(1-{[trans-4- (4-morpholinyl carbonyl)cyclohexyl] methyl}-4-piperidinyl) oxy]quinoline | hexyl | 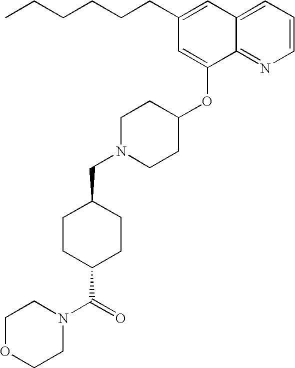 | 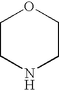 | 2.71 | 522 |
| 58 | trans-4-({4-[(6-hexyl-8- quinolinyl)oxy]-1- piperidinyl}methyl)-N- [3-(methyloxy) propyl]cyclohexane carboxamide | hexyl | 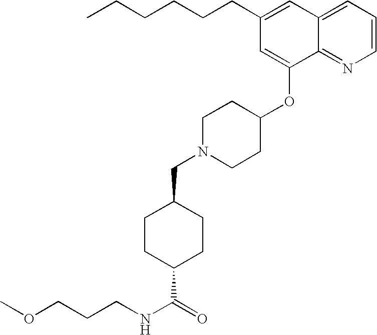 | 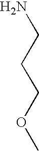 | 2.73 | 524 |
| 59 | trans-4-({4-[(6-butyl-8- quinolinyl)oxy]-1- piperidinyl}methyl)-N- (4-methylphenyl) cyclohexane carboxamide | butyl | 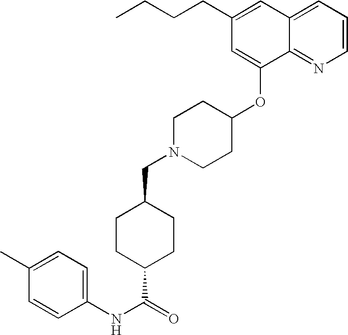 | 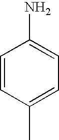 | 2.81 | 514 |
| 60 | trans-4-({4-[(6-butyl-8- quinolinyl)oxy]-1- piperidinyl}methyl)-N- [(4-methylphenyl) methyl]cyclohexane carboxamide | butyl | 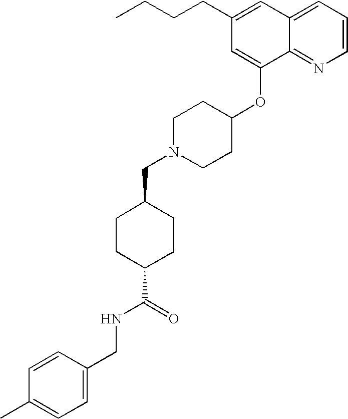 | 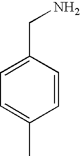 | 2.79 | 528 |
| 61 | trans-4-({4-[(6-butyl-8- quinolinyl)oxy]-1- piperidinyl}methyl)-N- (3-methylbutyl) cyclohexane carboxamide | butyl | 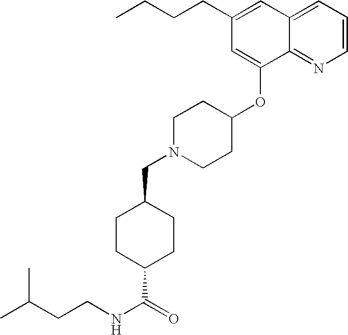 | 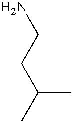 | 2.7 | 494 |
| 62 | 6-butyl-8-[(1-{[trans-4- (1-pyrrolidinyl carbonyl)cyclohexyl] methyl}-4-piperidinyl) oxy]quinoline | butyl | 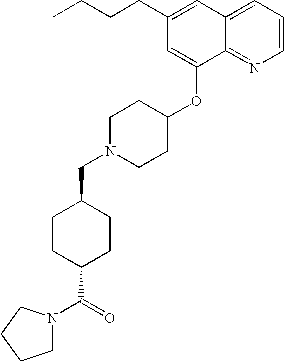 | 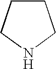 | 2.48 | 478 |
| 63 | 6-butyl-8-[(1-{[trans-4- (4-morpholinyl carbonyl)cyclohexyl] methyl}-4-piperidinyl) oxy]quinoline | butyl | 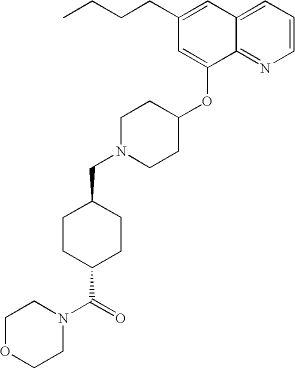 | 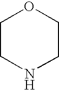 | 2.42 | 494 |

### Example 64

### N-(4-{4-[(6-Butyl-8-quinolinyl)oxy]-1-piperidinyl}butyl)propanamide, dihydrochloride salt

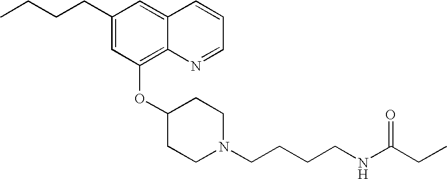

To a solution of (4-{4-[(6-butyl-8-quinolinyl)oxy]-1-piperidinyl}butyl)amine (for example, as prepared for Intermediate 44) (29 mg, 0.081 mmol) in dry DCM (1 ml) was added triethylamine (0.034 ml, 0.245 mmol). This solution was stirred at room temperature for 5 min, before adding propionyl chloride (commercially available, for example, from Aldrich) (0.014 ml, 0.162 mmol). The resultant solution was left to stand at room temperature for 3 days. MeOH (0.5 ml) was added and the reaction mixture applied to an SCX-2 cartridge (10 g, pre-conditioned with MeOH), washed with MeOH (×3) and then eluted with 10% aqueous ammonia in MeOH (×4). The solvents were removed in vacuo, and the resultant residue was dissolved in MeOH (0.5 ml) and treated with methanolic hydrogen chloride (1.25 M solution, 0.5 ml). After 30 min the solvent was removed to afford the title compound (40 mg). LCMS RT=2.72 min, ES+ve m/z 412 [M+H]⁺

### Examples 65 to 78

Examples 65 to 78 were prepared in an analogous manner to Example 64

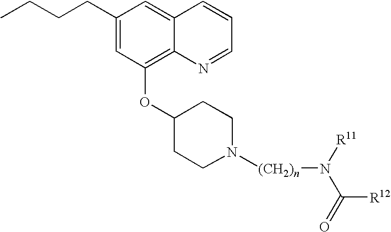

It will be appreciated that (CH₂)ₙ above corresponds to the straight chain C₁₋₆alkylene of R¹⁰.

The following acid chlorides were used:

Propionyl chloride, butanoyl chloride, 2-methylpropanoyl chloride, 2,2-dimethylpropanoyl chloride, cyclohexanecarbonyl chloride (all of which are commercially available, for example, from Aldrich).

If n=2, the amine used was (2-{4-[(6-butyl-8-quinolinyl)oxy]-1-piperidinyl}ethyl)amine (for example, as prepared for Intermediate 39)

If n=3, the amine used was (3-{4-[(6-butyl-8-quinolinyl)oxy]-1-piperidinyl}propyl)amine (for example, as prepared for Intermediate 43)

If n=4, the amine used (4-{4-[(6-butyl-8-quinolinyl)oxy]-1-piperidinyl}butyl)amine (for example, as prepared for Intermediate 44)

If n=5, amine used was (5-{4-[(6-butyl-8-quinolinyl)oxy]-1-piperidinyl}pentyl)amine (for example, as prepared for Intermediate 45)

| | | | | | Acid | LCMS | |
|---|---|---|---|---|---|---|---|
| Example | | | | | Chloride | RT | ES + ve |
| No. | Compound Name | R¹¹ | n = | Structure | (R¹²C(O)Cl) | (min) | m/z |
| 65 | N-(4-{4-[(6-butyl-8- quinolinyl)oxy]-1- piperidinyl}butyl) butanamide | H | 4 | 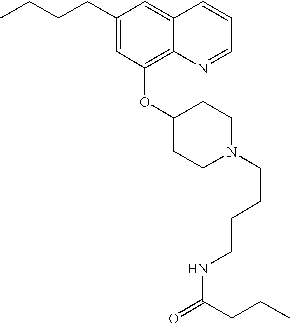 | 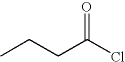 | 2.79 | 426 |
| 66 | N-(4-{4-[(6-butyl-8- quinolinyl)oxy]-1- piperidinyl}butyl)-2- methylpropanamide | H | 4 | 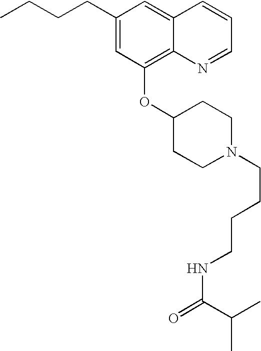 | 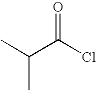 | 2.79 | 426 |
| 67 | N-(4-{4-[(6-butyl-8- quinolinyl)oxy]-1- piperidinyl}butyl)- 2,2-dimethyl propanamide | H | 4 | 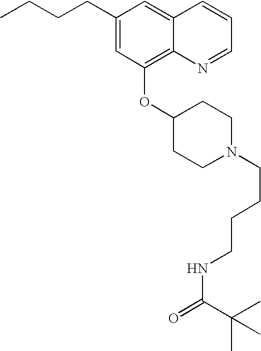 | 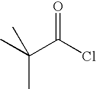 | 2.89 | 440 |
| 68 | N-(3-{4-[(6-butyl-8- quinolinyl)oxy]-1- piperidinyl}propyl)- 2-methyl propanamide | H | 3 | 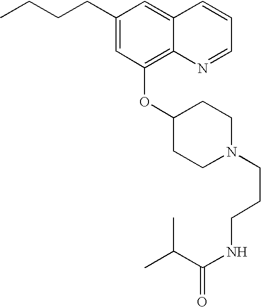 | 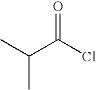 | 2.70 | 412 |
| 69 | N-(3-{4-[(6-butyl-8- quinolinyl)oxy]-1- piperidinyl}propyl)- 2,2-dimethyl propanamide | H | 3 | 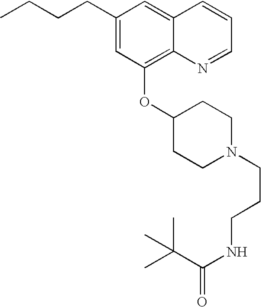 | 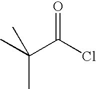 | 2.84 | 426 |
| 70 | N-(3-{4-[(6-butyl-8- quinolinyl)oxy]-1- piperidinyl}propyl) butanamide | H | 3 | 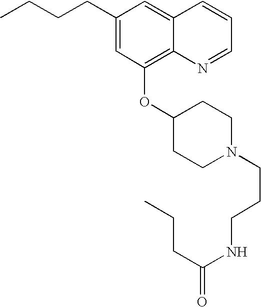 | 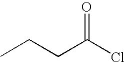 | 2.69 | 412 |
| 71 | N-(2-{4-[(6-butyl-8- quinolinyl)oxy]-1- piperidinyl}ethyl)-2- methylpropanamide | H | 2 | 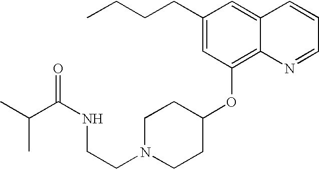 | 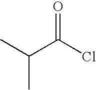 | 2.74 | 398 |
| 72 | N-(2-{4-[(6-butyl-8- quinolinyl)oxy]-1- piperidinyl}ethyl)- 2,2-dimethyl propanamide | H | 2 | 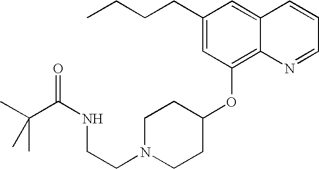 | 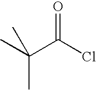 | 2.85 | 412 |
| 73 | N-(2-{4-[(6-butyl-8- quinolinyl)oxy]-1- piperidinyl}ethyl) propanamide | H | 2 | 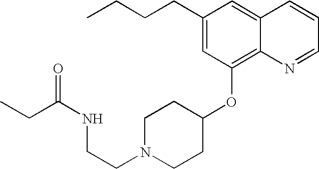 | 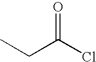 | 2.59 | 384 |
| 74 | N-(2-{4-[(6-butyl-8- quinolinyl)oxy]-1- piperidinyl}ethyl) butanamide | H | 2 | 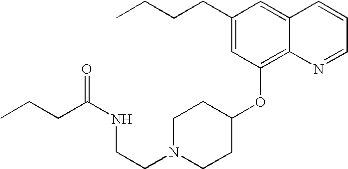 | 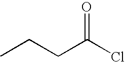 | 2.68 | 398 |
| 75 | N-(2-{4-[(6-butyl-8- quinolinyl)oxy]-1- piperidinyl}ethyl) cyclohexane carboxamide | H | 2 | 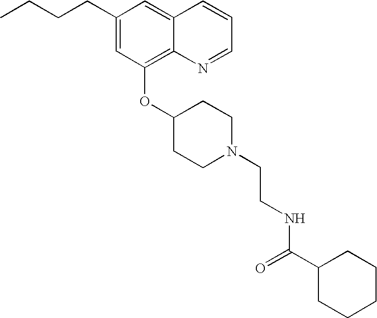 | 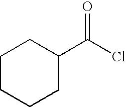 | 3.12 | 438 |
| 76 | N-(5-{4-[(6-butyl-8- quinolinyl)oxy]-1- piperidinyl}pentyl)- 2,2-dimethyl propanamide | H | 5 | 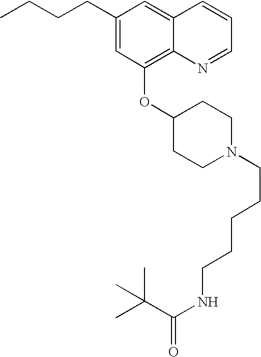 | 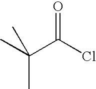 | 2.99 | 454 |
| 77 | N-(5-{4-[(6-butyl-8- quinolinyl)oxy]-1- piperidinyl}pentyl) propanamide | H | 5 | 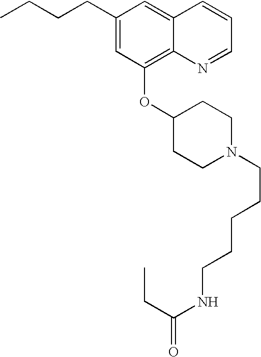 | 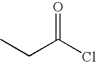 | 2.78 | 426 |
| 78 | N-(5-{4-[(6-butyl-8- quinolinyl)oxy]-1- piperidinyl}pentyl) cyclohexane carboxamide | H | 5 | 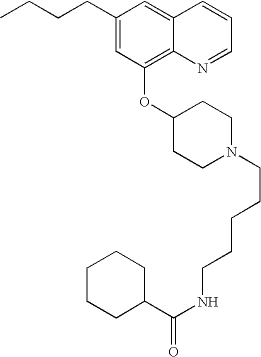 | 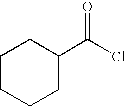 | 3.01 | 480 |

### Example 79

### N-(4-{4-[(6-Butyl-8-quinolinyl)oxy]-1-piperidinyl}butyl)acetamide, dihydrochloride salt

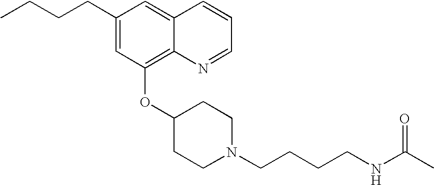

To a solution of acetic acid (commercially available, for example, from Aldrich) (9 mg, 0.15 mmol) in dry DMF (0.5 ml) was added TBTU (64 mg, 0.2 mmol). The reaction mixture was stirred at room temperature for 10 min before adding (4-{4-[(6-butyl-8-quinolinyl)oxy]-1-piperidinyl}butyl)amine (for example, as prepared for Intermediate 44), (25 mg, 0.07 mmol) followed with triethylamine (0.064 ml, 0.46 mmol). The resultant pale yellow solution was stirred for 3 h. The reaction mixture was then applied onto an SCX-2 cartridge (10 g, pre-conditioned with MeOH), washed with MeOH (×3) and then eluted with 10% aqueous ammonia in MeOH (×4). The solvents were removed and the resultant residue purified by MDAP HPLC. After removal of the solvent the residue was treated with methanolic hydrogen chloride (1.25 M, 0.5 ml). After 10 min the solvent was removed to afford the title compound (16 mg). LCMS RT=2.53 min, ES+ve m/z 398 [M+H]⁺

### Examples 80 to 83

### Examples 80 to 83 were prepared in an analogous manner to Example 79

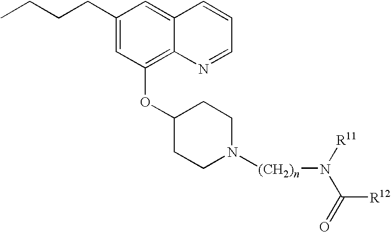

It will be appreciated that (CH₂)ₙ above corresponds to the straight chain C₁₋₆alkylene of R¹⁰.

If n=2, the amine used was (2-{4-[(6-butyl-8-quinolinyl)oxy]-1-piperidinyl}ethyl)amine (for example, as prepared for Intermediate 39).

If n=3, the amine used was (3-{4-[(6-butyl-8-quinolinyl)oxy]-1-piperidinyl}propyl)amine (for example, as prepared for Intermediate 43)

The following acids were used:

3-(Methoxy)propanoic acid, (commercially available, for example, from Aldrich) and 4-(methyloxy)butanoic acid, (for example, as prepared for Intermediate 46).

| | | | | | Acid | LCMS | |
|---|---|---|---|---|---|---|---|
| Example | | | | | Chloride | RT | ES + ve |
| No. | Compound Name | R¹¹ | n = | Structure | (R¹²C(O)Cl) | (min) | m/z |
| 80 | N-(2-{4-[(6-butyl-8- quinolinyl)oxy]-1- piperidinyl}ethyl)-3- (methyloxy) propanamide | H | 2 | 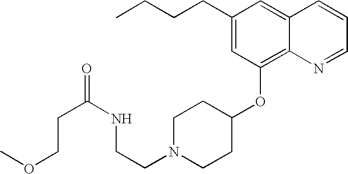 | 3- (methoxy) propanoic acid | 2.53 | 414 |
| 81 | N-(2-{4-[(6-butyl-8- quinolinyl)oxy]-1- piperidinyl}ethyl)-4- (methyloxy) butanamide | H | 2 | 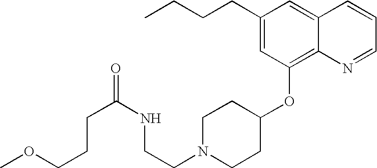 | 4- (Methoxy) butanoic acid | 2.65 | 428 |
| 82 | N-(3-{4-[(6-butyl-8- quinolinyl)oxy]-1- piperidinyl}propyl)- 3-(methyloxy) propanamide | H | 3 | 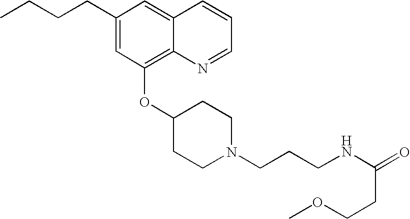 | 3- (Methoxy) propanoic acid | 2.62 | 428 |
| 83 | N-(3-{4-[(6-butyl-8- quinolinyl)oxy]-1- piperidinyl}propyl)- 4-(methyloxy) butanamide | H | 3 | 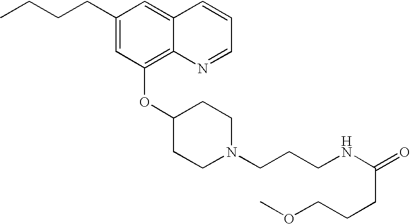 | 4- (Methoxy) butanoic acid | 2.65 | 442 |

### Example 84

### N-(4-{4-[(6-butyl-8-quinolinyl)oxy]-1-piperidinyl}butyl)-4-(methyloxy) butanamide, formate salt,

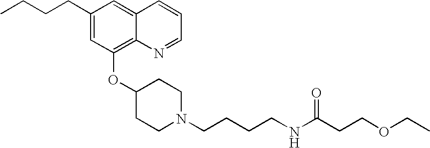

To a solution of 4-(methyloxy)butanoic acid (for example, as prepared for Intermediate 46), (13 mg, 0.11 mmol) in dry DMF (0.5 ml) was added TBTU (35 mg, 0.11 mmol). The reaction mixture was stirred at room temperature for 10 min before adding (4-{4-[(6-butyl-8-quinolinyl)oxy]-1-piperidinyl}butyl)amine, (for example, as prepared for Intermediate 44) (23 mg, 0.065 mmol), followed by triethylamine (0.033 ml, 0.24 mmol). The resultant pale yellow solution was stirred overnight. The reaction mixture was then applied onto an SCX-2 cartridge (10 g, pre-conditioned with MeOH), washed with MeOH (×3) and then eluted with 10% aqueous ammonia in MeOH (×4). The solvents were removed in vacuo and the resultant residue purified by MDAP HPLC to afford the title compound (18 mg). LCMS RT=2.60 min, ES+ve m/z 456 [M+H]⁺

### Example 85

### N-(4-{4-[(6-butyl-8-quinolinyl)oxy]-1-piperidinyl}butyl)-3-(methyloxy) propanamide, formate salt, (1:1)

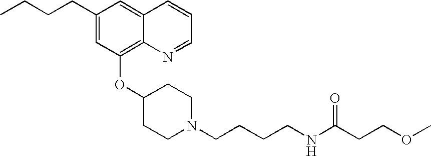

The title compound was prepared in an analogous manner to Example 85, using 3-methoxypropionic acid (commercially available, for example from Aldrich), and (4-{4-[(6-butyl-8-quinolinyl)oxy]-1-piperidinyl}butyl)amine (for example, as prepared for Intermediate 44). LCMS RT=2.51 min, ES+ve m/z 442 [M+H]⁺

### Example 86

### N-(3-{4-[(6-Butyl-8-quinolinyl)oxy]-1-piperidinyl}propyl)propanamide, dihydrochloride salt

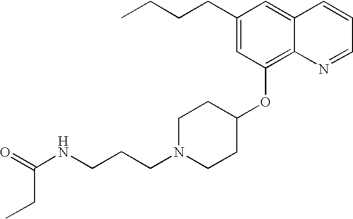

Propanoic acid (commercially available, for example, from Aldrich) (33 μl, 0.44 mmol), TBTU (156 mg, 0.49 mmol) and N,N′-diisopropylethylamine (230 μl, 1.32 mmol) were combined in dry DMF (2 ml) and stirred at 20° C. for 20 min. (3-{4-[(6-Butyl-8-quinolinyl)oxy]-1-piperidinyl}propyl)amine (for example, as prepared for Intermediate 43) (50 mg, 0.15 mmol) was added and stirring was continued for 5 h. The solvent was removed under a stream of nitrogen. The residue was applied to an aminopropyl SPE cartridge (5 g) and eluted with 10% MeOH in DCM. The solvents were removed in vacuo and the resultant residue purified by MDAP HPLC. After removal of the solvent, the residue was dissolved in methanolic hydrogen chloride (1.25 M, 2 ml), then re-concentrated under a stream of nitrogen at 40° C. to afford the title compound (31 mg, 44%). LCMS RT=2.30 min, ES+ve m/z 398 [M+H]⁺.

### Example 87

### 6-Butyl-8-({1-[3-(3-propyl-1,2,4-oxadiazol-5-yl)propyl]-4-piperidinyl}oxy)quinoline, dihydrochloride salt

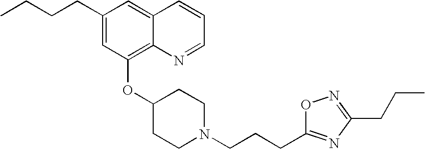

To a solution of 4-{4-[(6-butyl-8-quinolinyl)oxy]-1-piperidinyl}butanoic acid (for example, as prepared for Intermediate 28) (30 mg, 0.064 mmol) in dry DMF (0.5 ml) was added TBTU (0.025 g, 0.076 mmol) followed by triethylamine (0.018 ml, 0.128 mmol). The reaction mixture was stirred at room temperature for 10 min and then treated with N-hydroxybutanimidamide (commercially available, for example, from Apollo) (7 mg, 0.06 mmol). The resultant reaction mixture was stirred at room temperature for 1 h and then allowed to stand overnight. More TBTU (35 mg, 0.109 mmol) and triethylamine (0.020 ml, 0.14 mmol) followed by N-hydroxybutanimidamide (8 mg, 0.08 mmol) were added to the reaction mixture and the reaction was stirred for 1.5 h. More TBTU (40 mg, 0.12 mmol) and triethylamine (0.020 ml, 0.14 mmol) followed with N-hydroxybutanimidamide (10 mg, 0.1 mmol) were added to the reaction mixture and the reaction was stirred for a further 30 min. The reaction mixture was transferred to a microwave vial and heated in a SmithCreator™ microwave oven at 80° C. for 5 min. More triethylamine (0.030 ml) was added to the reaction and heating was continued in the microwave at 90° C. for another 5 min. This addition of triethylamine and further heating was repeated 7 times. The reaction mixture was then poured onto an SCX-2 cartridge (10 g, pre-conditioned with MeOH), washed with MeOH (×3) and eluted with 10% aqueous 0.88 s.g. ammonia in MeOH (×3). The required fraction was concentrated in vacuo and the resultant residue purified by MDAP HPLC and treated with methanolic hydrogen chloride (1.25 M, 0.3 ml). After 15 min the mixture was concentrated in vacuo to afford the title compound (6.5 mg). LCMS RT=3.06 min, ES+ve m/z 437 [M+H]⁺

### Example 88

### N-(1,1-dimethylethyl)-4-{4-[(6-pentyl-8-quinolinyl)oxy]-1-piperidinyl}butanamide, hydrochloride salt

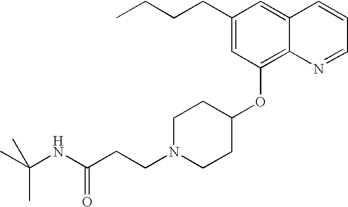

To a solution of 4-{4-[(6-pentyl-8-quinolinyl)oxy]-1-piperidinyl}butanoic acid, partial triethylamine salt (for example, as prepared for Intermediate 48) (39 mg, 0.09 mmol) in DMF (1 ml) was added a solution of TBTU (59 mg, 0.18 mmol) in DMF (1 ml). Triethylamine (31 μl, 0.23 mmol) was added and the mixture was stirred at room temperature for 15 min. Tert-butylamine (commercially available, for example, from Aldrich), (14 μl, 0.14 mmol) was added and the resulting mixture was stirred at room temperature overnight. The reaction mixture was applied to an SCX-2 ion exchange cartridge (20 g, pre-conditioned with MeOH). The cartridge was washed with methanol, and then eluted with 10% ammonia in MeOH. The relevant basic fractions were concentrated in vacuo. The residue was purified by MDAP HPLC. The appropriate fractions were combined and concentrated to give the title compound as the formate salt: LCMS RT=3.17 min, ES+ve m/z 440 (M-FH)⁺; ¹H NMR 6 (CD₃OD) 8.78 (1H, dd, J=4, 2 Hz), 8.42 (1H, s), 8.26 (1H, dd, J=8, 2 Hz), 7.53 (1H, dd, J=8, 4 Hz), 7.34 (1H, s), 7.20 (1H, d, J=1.5 Hz), 5.07-5.00 (1H, m), 3.72-3.62 (2H, m), 3.41-3.27 (2H, m, partially obscured by MeOD), 3.15 (2H, t, J=7.5 Hz), 2.79 (2H, t, J=8 Hz), 2.37 (2H, t, J=7 Hz), 2.32-2.23 (4H, m), 2.06-1.96 (2H, m), 1.78-1.68 (2H, m), 1.42-1.31 (13H, m), 0.91 (3H, t, J=7 Hz).

The material was dissolved in methanol and treated with 1.25 M hydrogen chloride in MeOH (0.5 ml). The volatiles were removed in vacuo to give the title compound as a yellow glass (10 mg, 22%): LCMS RT=3.18 min, ES+ve m/z 440 (M+H)⁺.

### Example 89

### N,N-Diethyl-4-{4-[(6-pentyl-8-quinolinyl)oxy]-1-piperidinyl}butanamide, hydrochloride salt

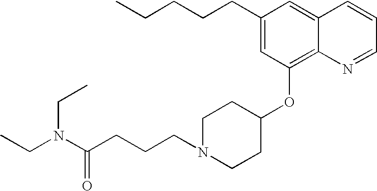

The title compound was prepared in an analogous manner to Example 88, using 4-{4-[(6-pentyl-8-quinolinyl)oxy]-1-piperidinyl}butanoic acid, partial triethylamine salt (for example, as prepared for Intermediate 48) (39 mg, 0.09 mmol), TBTU (56 mg, 0.17 mmol), triethylamine (31 μl, 0.23 mmol) and diethylamine (commercially available, for example, from Aldrich) (15 μl, 0.14 mmol) to give the title compound as the formate salt: LCMS RT=3.09 min, ES+ve m/z 440 (M-FH)⁺; ¹H NMR 6 (CD₃OD) 8.78 (1H, dd, J=4, 2 Hz), 8.35 (1.5H, s), 8.26 (1H, dd, J=8, 2 Hz), 7.53 (1H, dd, J=8, 4 Hz), 7.33 (1H, s), 7.21 (1H, d, J=1.5 Hz), 5.07-5.01 (1H, m), 3.73-3.64 (2H, m), 3.40 (6H, q, J=7 Hz), 3.20 (2H, t, J=7.5 Hz), 2.80 (2H, t, J=8 Hz), 2.61 (2H, t, J=7.5 Hz), 2.33-2.25 (4H, m), 2.10-2.05 (2H, m), 1.78-1.69 (2H, m), 1.42-1.33 (4H, m), 1.21 (3H, t, J=7 Hz), 1.13 (3H, t, J=7 Hz), 0.91 (3H, t, J=7 Hz).

The material was dissolved in methanol and treated with 1.25 M hydrogen chloride in MeOH (0.5 ml). The volatiles were removed in vacuo to give the title compound as a yellow glass (14 mg, 30%): LCMS RT=3.01 min, ES+ve m/z 440 (M+H)⁺.

### Example 90

### N-(3-{4-[(6-Pentyl-8-quinolinyl)oxy]-1-piperidinyl}propyl)propanamide, dihydrochloride salt

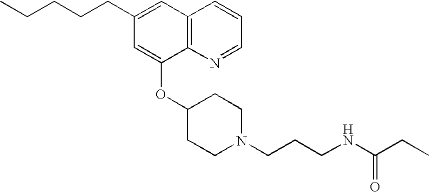

To a solution of (3-{4-[(6-pentyl-8-quinolinyl)oxy]-1-piperidinyl}propyl)amine (for example, as prepared for Intermediate 50) (38 mg, 0.11 mmol) in DCM (1 ml) was added triethylamine (18 μl, l, 0.13 mmol) and propionyl chloride (commercially available, for example, from Aldrich) (11 μl, 0.12 mmol). The resulting solution was stirred at room temperature for 1 h. MeOH (2 ml) was added and the reaction mixture was concentrated under a stream of nitrogen. The residue was purified by MDAP HPLC. The appropriate fractions were combined and concentrated to give the title compound as the formate salt: LCMS RT=2.86 min, ES+ve m/z 412 (M+H)⁺; ¹H NMR 6 (CD₃OD) 8.78 (1H, dd, J=4, 2 Hz), 8.30 (1.9H, br s), 8.27 (1H, dd, J=8, 2 Hz), 7.53 (1H, dd, 8, 4 Hz), 7.34 (1H, s), 7.20 (1H, d, J=1.5 Hz), 5.07-5.01 (1H, m), 3.72-3.63 (2H, m), 3.41-3.27 (4H, m, partially obscured by MeOD), 3.20-3.14 (2H, m), 2.79 (2H, t, J=8 Hz), 2.32-2.20 (6H, m), 2.02-1.93 (2H, m), 1.78-1.69 (2H, m), 1.43-1.33 (4H, m), 1.14 (3H, t, J=8 Hz), 0.91 (3H, t, J=7 Hz).

The material was dissolved in methanol and treated with 1.25 M hydrogen chloride in MeOH. The volatiles were removed in vacuo to give the title compound (40 mg, 75%): LCMS RT=2.88 min, ES+ve m/z 412 (M+H)⁺.

### Example 91

### 2,2-Dimethyl-N-(3-{4-[(6-pentyl-8-quinolinyl)oxy]-1-piperidinyl}propyl) propanamide, dihydrochloride salt

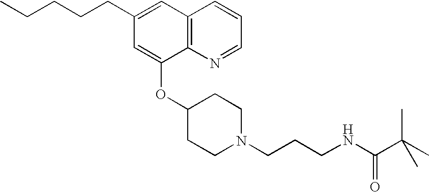

The title compound was prepared in an analogous manner to Example 90, using (3-{4-[(6-pentyl-8-quinolinyl)oxy]-1-piperidinyl}propyl)amine (for example, as prepared for Intermediate 50) (33 mg, 0.09 mmol), DCM (1 ml), triethylamine (18 μl, 0.13 mmol) and 2,2-dimethylpropanoyl chloride, (commercially available, for example, from Aldrich) (15 μl, 0.12 mmol), to give the title compound as the formate salt: LCMS RT=3.11 min, ES+ve m/z 440 (M+H)⁺; ¹H NMR 6 (CD₃OD) 8.78 (1H, dd, J=4, 2 Hz), 8.27 (1H, dd, J=8, 2 Hz), 8.24 (2.3H, br s), 7.53 (1H, dd, J=8, 4 Hz), 7.34 (1H, s), 7.21 (1H, d, J=1.5 Hz), 5.08-5.02 (1H, m), 3.73-3.64 (2H, m), 3.41-3.27 (4H, m, partially obscured by MeOD), 3.14 (2H, t, J=7.5 Hz), 2.80 (2H, t, J=8 Hz), 2.35-2.24 (4H, m), 2.02-1.93 (2H, m), 1.78-1.69 (2H, m), 1.43-1.33 (4H, m), 1.20 (9H, s), 0.91 (3H, t, J=7 Hz).

The material was dissolved in methanol and treated with 1.25 M hydrogen chloride in MeOH. The volatiles were removed in vacuo to give the title compound (23 mg, 50%): LCMS RT=3.08 min, ES+ve m/z 440 (M+H)⁺.

### Biological Assays

The compounds of the invention may be tested for in vitro and/or in vivo biological activity in accordance with the following or similar assays.

### H1 Receptor Cell Line Generation and FLIPR Assay Protocol

### 1. Generation of Histamine H1 Cell Line

The human H1 receptor is cloned using known procedures described in the literature [Biochem. Biophys. Res. Commun., 201(2):894 (1994)]. Chinese hamster ovary (CHO) cells stably expressing the human H1 receptor are generated according to known procedures described in the literature [Br. J. Pharmacol., 117(6):1071 (1996)].

### Histamine H1 Functional Antagonist Assay: Determination of Functional pKi Values

The histamine H1 cell line is seeded into non-coated black-walled clear bottom 384-well tissue culture plates in alpha minimum essential medium (Gibco/Invitrogen, cat no. 22561-021), supplemented with 10′)/0 dialysed foetal calf serum (Gibco/Invitrogen cat no. 12480-021) and 2 mM L-glutamine (Gibco/Invitrogen cat no 25030-024) and is maintained overnight at 5% CO₂, 37° C.

Excess medium is removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) is added to each well and the plates are incubated for 60 min at 5% CO₂, 37° C.

10 μl of test compound, diluted to the required concentration in Tyrodes buffer+probenecid (or 10 μl Tyrodes buffer+probenecid as a control) is added to each well and the plate is incubated for 30 min at 37° C., 5% CO₂. The plates are then placed into a FLIPR™(Molecular Devices, UK) to monitor cell fluorescence (λₑₓ=488 nm, λ_{EM}=540 nm) in the manner described in Sullivan et al., (In: Lambert DG (ed.), Calcium Signaling Protocols, New Jersey: Humana Press, 1999, 125-136) before and after the addition of 10 μl histamine at a concentration that results in the final assay concentration of histamine being EC₈₀.

Functional antagonism is indicated by a suppression of histamine induced increase in fluorescence, as measured by the FLIPR™ system (Molecular Devices). By means of concentration effect curves, functional affinities are determined using standard pharmacological mathematical analysis.

### Histamine H1 Functional Antagonist Assay: Determination of Antagonist pA2 and Duration

The histamine H1 receptor expressing CHO cells are seeded into non-coated black-walled clear bottom 96-well tissue culture plates as described above.

Following overnight culture, growth medium is removed from each well, washed with 200 μl PBS and is replaced with 50 μl loading dye (250 μM Brilliant Black, 1 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)). Cells are incubated for 45 min at 37° C. The loading buffer is removed and the cells are washed as above, and 90 μl of Tyrodes buffer+probenecid is added to each well. 10 μl of test compound, diluted to the required concentration in Tyrodes buffer+probenecid (or 10 μl Tyrodes buffer+probenecid as a control) is added to each well and the plate is incubated for 30 min at 37° C., 5% CO₂.

The plates are then placed into a FLIPR™ (Molecular Devices, UK) to monitor cell fluorescence (λₑₓ=488 nm, λ_{EM}=540 nm) in the manner described in Sullivan et al., (In: Lambert DG (ed.), Calcium Signaling Protocols, New Jersey: Humana Press, 1999, 125-136) before and after the addition of 50 μl histamine over a concentration range of 1 mM-0.1 nM. The resultant concentration response curves are analysed by non-linear regression using a standard four parameter logistic equation to determine the histamine EC₅₀, the concentration of histamine required to produce a response of 50% of the maximum response to histamine. The antagonist pA2 is calculated using the following standard equation: pA2=log(DR-1)-log[B] where DR=dose ratio, defined as EC₅₀antagonist-treated/EC₅₀control and [B]=concentration of antagonist.

To determine the antagonist duration, cells are cultured overnight in non-coated black-walled clear bottom 96-well tissue culture plates, are washed with PBS and are incubated with a concentration of antagonist chosen to give an approximate DR in the range 30-300. Following the 30 min antagonist incubation period, the cells are washed two or three times with 200 μl of PBS and then 100 μl Tyrodes buffer is added to each well to initiate antagonist dissociation. Following incubation for predetermined times, typically 30-270 min at 37° C., the cells are then washed again with 200 μl PBS and are incubated with 100 μl Tyrodes buffer containing Brilliant Black, probenecid and Fluo-4 for 45 min at 37° C., as described above. After this period, the cells are challenged with histamine in the FLIPR™ as described above. The dose ratio at each time point is used to determine the fractional H1 receptor occupancy by the following equation: fractional receptor occupancy=(DR-1)/DR. The decrease in receptor occupancy over time approximates to a straight line and is analysed by linear regression. The slope of this straight line fit is used as an index of the dissociation rate of the antagonist. The dose ratios for antagonist treated cells and for antagonist treated and washed cells at each time point are used to calculate a relative dose ratio (rel DR) which is also used as an index of antagonist duration. Antagonists with long duration of action produce rel DR values close to 1, and antagonists with short duration of action produce rel DR values that approaches the dose ratio value obtained for antagonist treatment alone.

### 2. H3 Receptor Cell Line Generation, Membrane Preparation and Functional GTPγS Assay Protocols

### Generation of Histamine H3 Cell Line

The histamine H3 cDNA is isolated from its holding vector, pcDNA3.1 TOPO (InVitrogen), by restriction digestion of plasmid DNA with the enzymes BamH1 and Not-1 and is ligated into the inducible expression vector pGene (InVitrogen) digested with the same enzymes. The GeneSwitch™ system (a system where in transgene expression is switched off in the absence of an inducer and switched on in the presence of an inducer) is performed as described in U.S. Pat. Nos. 5,364,791; 5,874,534; and 5,935,934. Ligated DNA is transformed into competent DH5α E. coli host bacterial cells and is plated onto Luria Broth (LB) agar containing Zeocin™ (an antibiotic which allows the selection of cells expressing the sh ble gene which is present on pGene and pSwitch) at 50 μgml⁻¹. Colonies containing the re-ligated plasmid are identified by restriction analysis. DNA for transfection into mammalian cells is prepared from 250 ml cultures of the host bacterium containing the pGeneH3 plasmid and is isolated using a DNA preparation kit (Qiagen Midi-Prep) as per manufacturers guidelines (Qiagen).

CHO K1 cells previously transfected with the pSwitch regulatory plasmid (InVitrogen) are seeded at 2×10⁶ cells per T75 flask in Complete Medium, containing Hams F12 (GIBCOBRL, Life Technologies) medium supplemented with 10% v/v dialysed foetal bovine serum, L-glutamine, and hygromycin (100 μgml⁻¹), 24 h prior to use. Plasmid DNA is transfected into the cells using Lipofectamine plus according to the manufacturer's guidelines (InVitrogen). 48 h post transfection, cells are placed into complete medium supplemented with 500 μgml⁻¹ Zeocin™.

10-14 days post selection, 10 nM Mifepristone (InVitrogen) is added to the culture medium to induce the expression of the receptor. 18 h post induction, cells are detached from the flask using ethylenediamine tetra-acetic acid (EDTA; 1:5000; InVitrogen), following several washes with PBS, pH 7.4 and are resuspended in Sorting Medium containing Minimum Essential Medium (MEM), without phenol red, and are supplemented with Earles salts and 3% Foetal Clone II (Hyclone). Approximately 1×10⁷ cells are examined for receptor expression by staining with a rabbit polyclonal antibody, 4a, raised against the N-terminal domain of the histamine H3 receptor, are incubated on ice for 60 min, followed by two washes in sorting medium. Receptor bound antibody is detected by incubation of the cells for 60 min on ice with a goat anti rabbit antibody, conjugated with Alexa 488 fluorescence marker (Molecular Probes). Following two further washes with Sorting Medium, cells are filtered through a 50 μm Filcon™ (BD Biosciences) and then are analysed on a FACS Vantage SE Flow Cytometer fitted with an Automatic Cell Deposition Unit. Control cells are non-induced cells treated in an analogous manner. Positively stained cells are sorted as single cells into 96-well plates, containing Complete Medium containing 500 μgml⁻¹ Zeocin™ and are allowed to expand before reanalysis for receptor expression via antibody and ligand binding studies. One clone, 3H3, is selected for membrane preparation.

Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10⁻⁶ M leupeptin (acetyl-leucyl-leucyl-arginal; Sigma L2884), 25 μgml⁻¹ bacitracin (Sigma B0125), 1 mM phenylmethylsulfonyl fluoride (PMSF) and 2×10⁻⁶ M pepstain A (Sigma)). The cells are then homogenised by 2×15 sec bursts in a 1 L glass Waring blender, followed by centrifugation at 500 g for 20 min. The supernatant is then spun at 48,000 g for 30 min. The pellet is resuspended in homogenisation buffer (4× the volume of the original cell pellet) by vortexing for 5 sec, followed by homogenisation in a Dounce homogeniser (10-15 strokes). At this point the preparation is aliquoted into polypropylene tubes and stored at −80° C.

### Histamine H3 Functional Antagonist Assay

For each compound being assayed, in a solid white 384 well plate, is added:—

(a) 0.5 μl of test compound diluted to the required concentration in DMSO (or 0.5 μl DMSO as a control);
(b) 30 μl bead/membrane/GDP mix which is prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-hydroxyethylpiperazine-N′-2-ethanesulfonic acid (HEPES)+100 mM NaCl+10 mM MgCl₂, pH 7.4 NaOH) to give a final volume of 30 μl which contains 5 μg protein, 0.25 mg bead per well and 10 μM final assay concentration of guanosine 5′diphosphate (GDP) (Sigma, diluted in assay buffer) incubating at room temperature for 60 min on a roller;
(c) 15 μl 0.38 nM [³⁵S]-GTPγS (Amersham; Radioactivity concentration=37 MBqml⁻¹; Specific activity=1160 Cimmol⁻¹), histamine (at a concentration that results in the final assay concentration of histamine being EC₈₀).

After 2-6 h, the plate is centrifuged for 5 min at 1500 rpm and counted on a Viewlux counter using a 613/55 filter for 5 minplate⁻¹. Data is analysed using a 4-parameter logistic equation. Basal activity is used as minimum, i.e. histamine not added to well.

### Results

In these or similar biological assays, the following data were obtained:

(i) The compound of Example 10 had an average pKi (pKb) at H1 of less than approximately 6.0. The compound of Examples 38 and 40 had an average pKi (pKb) at H1 of approximately 6.0. The compound of Examples 1-4, 8, 9, 11-15, 24, 26, 29, 30, 41, 42, 46, 48-55, 57-60, 63, 66, 83 and 89 had an average pKi (pKb) at H1 of greater than approximately 7.0. The compound of Examples 5-7, 16-23, 25, 27, 28, 31-37, 39, 43-45, 47, 56, 61, 62, 64, 65, 67-82, 84-88, 90, 91 had an average pKi (pKb) at H1 of greater than approximately 8.0.

The compounds of Examples 38, 40 and 41 had average pA2 values of greater than approximately 7. The compounds of Examples 1-5, 9, 11, 12, 14, 23, 31, 32-37, 39, 42, 45, 46, 47, 64, 69, 71, 73-75, 79-82, 84, 86, 89 and 91 had average pA2 values of greater than approximately 8. The compounds of Examples 6, 7, 17-19, 22, 24, 25, 27, 30, 43, 44, 65-68, 70, 72, 76, 77, 78, 83, 85, 87 and 90 had average pA2 values of greater than approximately 9.

(ii) The compounds of Examples 8, 9, 11-17 and 19-31 had an average pKi (pKb) at H3 of less than approximately 5.5. The compounds of Examples 1-7, 10, 18, 33-37 and 39-91 had an average pKi (pKb) at H3 of less than approximately 6.5. The compound of Example 38 had an average pKi (pKb) at H3 of approximately 7.0.

(iii) The compounds of Examples 43, 44 and 72 exhibited at one or more time points a longer duration of action than azelastine in the histamine H1 functional antagonist assay. Other compounds were either not tested or were tested and did not exhibit a longer duration of action.

1. A compound of formula (I) 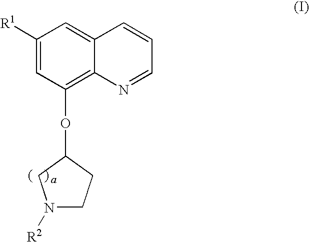
wherein
R¹ represents straight chain C₁₋₆alkyl;
a represents 1 or 2;
R² represents a group of formula (i), (ii), (iii), (iv) or (v) below 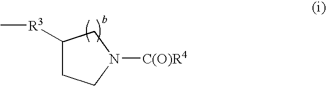
in which R³ represents methylene or ethylene;
b represents 1 or 2; and
R⁴ represents a group selected from —C₁₋₆alkyl (optionally substituted by up to three substituents independently selected from halogen or hydroxy); —C₁₋₆alkylene-O—C₁₋₆alkyl (optionally substituted by up to three substituents independently selected from by halogen and hydroxy); aryl (optionally substituted by up to three substituents independently selected from halogen, C₁₋₃alkyl, trifluoromethyl, and cyano); or —C₁₋₆alkylenearyl (in which the C₁₋₆alkylene is a straight chain and is optionally substituted by up to three substituents independently selected from C₁₋₃alkyl, halogen and hydroxy, and the aryl is optionally substituted by up to three substituents independently selected from halogen, C₁₋₃alkyl, trifluoromethyl, and cyano); 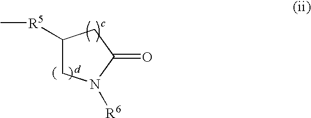
in which R⁵ represents methylene or ethylene;
c represents 0 or 1 and d represents 2 or 3, or c represents 2 or 3 and d represents 0 or 1; and
R⁶ represents hydrogen or C₁₋₆alkyl; 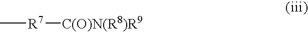
in which R⁷ represents a straight chain C₁₋₆alkylene (optionally substituted by one or two C₁₋₃alkyl), or —CH₂—C₅₋₆cycloalkyl;
R⁸ represents hydrogen or C₁₋₆alkyl;
R⁹ represents a group selected from hydrogen; —C₁₋₆alkyl (optionally substituted by up to three substituents independently selected from halogen and hydroxy); —C₁₋₆alkylene-O—C₁₋₆alkyl (optionally substituted by up to three substituents independently selected from halogen and hydroxy); C₃₋₇cycloalkyl (optionally substituted by up to three substituents independently selected from halogen, hydroxy and C₁₋₃alkyl); —C₁₋₃alkyleneC₃₋₇cycloalkyl (in which the C₁₋₆alkylene is straight chain and optionally substituted by up to three substituents independently selected from C₁₋₃alkyl, halogen and hydroxy, and the C₃₋₇cycloalkyl is optionally substituted by up to three substituents independently selected from halogen, hydroxy and C₁₋₃alkyl); aryl (optionally substituted by up to three substituents independently selected from halogen, C₁₋₃alkyl, trifluoromethyl, and cyano);
or —C₁₋₆alkylenearyl (in which the C₁₋₆alkylene is straight chain and is optionally substituted by up to three substituents independently selected from C₁₋₃alkyl, halogen or hydroxy, and the aryl is optionally substituted by up to three substituents independently selected from halogen, C₁₋₃alkyl, trifluoromethyl, and cyano);
or R⁸ and R⁹ together with the N atom to which they are attached represent a 5 to 7 membered saturated heterocyclic ring optionally containing one or two further heteroatoms independently selected from O and S; 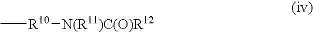
in which R¹ represents a straight chain C₁₋₆alkylene (optionally substituted by one or two C₁₋₃alkyl), or —CH₂—C₅₋₆cycloalkyl;
R¹¹ represents hydrogen or C₁₋₆alkyl; and
R¹² represents a group selected from C₁₋₆alkyl (optionally substituted up to three substituents independently selected from halogen and hydroxy); —C₁₋₆alkylene-O—C₁₋₆alkyl (optionally substituted up to three substituents independently selected from halogen and hydroxy); C₃₋₇cycloalkyl (optionally substituted by up to three substituents independently selected from halogen, hydroxy and C₁₋₃alkyl); —C₁₋₃alkyleneC₃₋₇cycloalkyl (in which the C₁₋₆alkylene is straight chain and optionally substituted by up to three substituents independently selected from C₁₋₃alkyl, halogen and hydroxy, and the C₃₋₇cycloalkyl is optionally substituted by up to three substituents independently selected from halogen, hydroxy and C₁₋₃alkyl); aryl (optionally substituted by up to three substituents independently selected from halogen, C₁₋₃alkyl, trifluoromethyl, and cyano); —C₁₋₆alkylenearyl (in which the C₁₋₆alkylene is straight chain and is optionally substituted by up to three substituents independently selected from C₁₋₃alkyl, halogen or hydroxy, and the aryl is optionally substituted by up to three substituents independently selected from halogen, C₁₋₃alkyl, trifluoromethyl, and cyano); 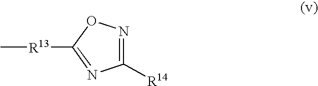
in which R¹³ represents a straight chain C₁₋₆alkylene (optionally substituted by one or two C₁₋₃alkyl), or —CH₂—C₅₋₆cycloalkyl; and
R¹⁴ represents a group selected from hydrogen; —C₁₋₆alkyl (optionally substituted by up to three substituents independently selected from halogen or hydroxy); —C₁₋₆alkylene-O—C₁₋₆alkyl (optionally substituted by up to three substituents independently selected from by halogen and hydroxy); aryl (optionally substituted by up to three substituents independently selected from halogen, C₁₋₃alkyl, trifluoromethyl, and cyano); or —C₁₋₆alkylenearyl (in which the C₁₋₆alkylene is a straight chain and is optionally substituted by up to three substituents independently selected from C₁₋₃alkyl, halogen and hydroxy, and the aryl is optionally substituted by up to three substituents independently selected from halogen, C₁₋₃alkyl, trifluoromethyl, and cyano);
or a salt thereof.

2. A compound according to claim 1 in which R¹ represents straight chain C₄₋₆alkyl;
a represents 1 or 2;
R³ represents methylene or ethylene;
b represents 2;
R⁴ represents a group selected from —C₁₋₆alkyl; C₄₋₇cycloalkyl; aryl (optionally substituted by one or two substituents independently selected from halogen, C₁₋₃alkyl, trifluoromethyl, and cyano); or —C₁₋₃alkylenearyl, (in which the C₁₋₃alkylene is a straight chain, and the aryl is optionally substituted by one or two substituents independently selected from halogen, C₁₋₃alkyl, trifluoromethyl, and cyano);
R⁵ represents methylene;
c represents 0 and d represents 3;
R⁶ represents hydrogen or C₁₋₃alkyl;
R⁷ represents a straight chain C₁₋₆alkylene (optionally substituted by one methyl), or —CH₂-cyclohexyl;
R⁸ represents hydrogen or C₁₋₃alkyl;
R⁹ represents a group selected from —C₁₋₆alkyl; C₄₋₇cycloalkyl; aryl (optionally substituted by one or two substituents independently selected from halogen, C₁₋₃alkyl, trifluoromethyl, and cyano); or —C₁₋₃alkylenearyl, (in which the C₁₋₃alkylene is a straight chain, and the aryl is optionally substituted by one or two substituents independently selected from halogen, C₁₋₃alkyl, trifluoromethyl, and cyano);
or R⁸ and R⁹ together represent a 5 or 6 membered saturated heterocyclic ring optionally containing one further heteroatom selected from O and S;
R¹⁰ represents a straight chain C₁₋₆alkylene (optionally substituted by one methyl group);
R¹¹ represents hydrogen or C₁₋₃alkyl;
R¹² represents a group selected from —C₁₋₆alkyl; C₄₋₇cycloalkyl; aryl (optionally substituted by one or two substituents independently selected from halogen, C₁₋₃alkyl, trifluoromethyl, and cyano); or —C₁₋₃alkylenearyl, (in which the C₁₋₃alkylene is a straight chain, and the aryl is optionally substituted by one or two substituents independently selected from halogen, C₁₋₃alkyl, trifluoromethyl, and cyano);
R¹³ represents a straight chain C₁₋₆alkylene (optionally substituted by one methyl group);
R¹⁴ represents a group selected from —C₁₋₆alkyl; C₄₋₇cycloalkyl; aryl (optionally substituted by one or two substituents independently selected from halogen, C₁₋₃alkyl, trifluoromethyl, and cyano); or —C₁₋₃alkylenearyl, (in which the C₁₋₃alkylene is a straight chain, and the aryl is optionally substituted by one or two substituents independently selected from halogen, C₁₋₃alkyl, trifluoromethyl, and cyano).

3. A compound according to claim 1 in which R¹ represents straight chain C₄₋₆alkyl.

4. A compound according to claim 1 in which a represents 2.

5. A compound according to claim 1 in which a represents 1.

6-8. (canceled)

9. A compound according to claim 1 in which R² represents a group of formula (iii) in which R⁷ represents a straight chain C₁₋₄alkylene (optionally substituted by one methyl group) or —CH₂-cyclohexyl;
R⁹ represents hydrogen or C₁₋₃alkyl;
R⁹ represents a group selected from —C₁₋₆alkyl —C₁₋₆alkylene-O—C₁₋₃alkyl; C₄₋₇cycloalkyl;
phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano);
or R⁸ and R⁹ together represent a 5 or 6 membered saturated heterocyclic ring optionally containing one further heteroatom selected from O and S.

10. A compound according to claim 1 in which R² represents a group of formula (Iv) in which R¹⁰ represents a straight chain C₁₋₄alkylene (optionally substituted by one methyl group);
R¹¹ represents hydrogen or C₁₋₃alkyl; and
R¹² represents a group selected from —C₁₋₆alkyl C₄₋₇cycloalkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano).

11. (canceled)

12. A compound according to claim 1 in which R⁸ represents hydrogen or C₁₋₃alkyl;
R⁹ represents a group selected from —C₁₋₆alkyl —C₁₋₃alkylene-O—C₁₋₃alkyl; phenyl (optionally substituted by one substituent selected from halogen, methyl, trifluoromethyl, and cyano); or C₁₋₃alkylenephenyl (in which the C₁₋₃alkylene is a straight chain, and the aryl is optionally substituted by one substituent selected from halogen, methyl, trifluoromethyl, and cyano); or
R⁸ and R⁹ together represent a 5 or 6 membered saturated heterocyclic ring optionally containing one further heteroatom selected from O and S.

13. A compound according to claim 1 which is:
8-({1-[(1-acetyl-4-piperidinyl)methyl]-4-piperidinyl}oxy)-6-butylquinoline;
6-butyl-8-({1-[(1-propanoyl-4-piperidinyl)methyl]-4-piperidinyl}oxy)quinoline;
8-({1-[2-(1-acetyl-4-piperidinyl)methyl]-4-piperidinyl}oxy)-6-butylquinoline;
6-butyl-8-({1-[(1-propanoyl-4-piperidinyl)methyl]-4-piperidinyl}oxy)quinoline;
3-({4-[(6-butyl-8-quinolinyl)oxy]-1-piperidinyl}methyl)-2-piperidinone;
4-{4-[(6-butyl-8-quinolinyl)oxy]-1-piperidinyl}-N-(1,1-dimethylethyl)butanamide;
3-{4-[(6-butyl-8-quinolinyl)oxy]-1-piperidinyl}-N,N-diethylpropanamide;
2-{4-[(6-butyl-8-quinolinyl)oxy]-1-piperidinyl}-N-[3-(methyloxy)propyl]acetamide;
2-{4-[(6-butyl-8-quinolinyl)oxy]-1-piperidinyl}-N,N-diethylacetamide;
2-{4-[(6-butyl-8-quinolinyl)oxy]-1-piperidinyl}-N-propylacetamide;
2-{4-[(6-butyl-8-quinolinyl)oxy]-1-piperidinyl}-N-[2-(methyloxy)ethyl]acetamide;
2-{4-[(6-butyl-8-quinolinyl)oxy]-1-piperidinyl}-N-(1,1-dimethylethyl)acetamide;
6-butyl-8-({1-[2-oxo-2-(1-piperidinyl)methyl]-4-piperidinyl}oxy)quinoline;
2-{4-[6-butyl-8-quinolinyl)oxy]-1-piperidinyl}-N-(1-methylethyl)acetamide;
3-{4-[6-butyl-8-quinolinyl)oxy]-1-piperidinyl}-N-ethylpropanamide;
3-{4-[6-butyl-8-quinolinyl)oxy]-1-piperidinyl}-N-[3-(methyloxy)propyl]propanamide;
3-{4-[(6-butyl-8-quinolinyl)oxy]-1-piperidinyl}-N-(1,1-dimethylethyl)propanamide;
6-butyl-8-({1-[3-oxo-3-(1-piperidinyl)propyl]-4-piperidinyl}oxy)quinoline;
3-{4-[(6-butyl-8-quinolinyl)oxy]-1-piperidinyl}-N-(1-methylethyl)propanamide;
4-{4-[(6-butyl-8-quinolinyl)oxy]-1-piperidinyl}-N-ethylbutanamide;
4-{4-[(6-butyl-8-quinolinyl)oxy]-1-piperidinyl}-N-[3-(methyloxy)propyl]butanamide;
4-{4-[(6-butyl-8-quinolinyl)oxy]-1-piperidinyl}-N-propylbutanamide;
4-{4-[(6-butyl-8-quinolinyl)oxy]-1-pipendinyl}-N-[2-(methyloxy)ethyl]butanamide;
6-butyl-8-({1-[4-oxo-4-(1-piperidinyl)butyl]-4-piperidinyl}oxy)quinoline;
4-{4-[(6-butyl-8-quinolinyl)oxy]-1-pipendinyl}-N-(1-methylethyl)butanamide;
5-{4-[(6-butyl-8-quinolinyl)oxy]-1-pipendinyl}-N-[3-(methyloxy)propyl]pentanamide;
5-{4-[(6-butyl-8-quinolinyl)oxy]-1-pipendinyl}-N,N-diethylpentanamide;
5-{4-[(6-butyl-8-quinolinyl)oxy]-1-pipendinyl}-N-propylpentanamide;
5-{4-[(6-butyl-8-quinolinyl)oxy]-1-pipendinyl}-N-[2-(methyloxy)ethyl]pentanamide;
5-{4-[(6-butyl-8-quinolinyl)oxy]-1-pipendinyl}-N-(1,1-dimethylethyl)pentanamide;
6-butyl-8-({1-[5-oxo-5-(1-piperidinyl)pentyl]-4-piperidinyl}oxy)quinoline;
5-{4-[(6-butyl-8-quinolinyl)oxy]-1-pipendinyl}-N-(1-methylethyl)pentanamide;
6-butyl-8-({(3R)-1-[3-oxo-3-(1-piperidinyl)propyl]-3-pyrrolidinyl}oxy)quinoline;
6-butyl-8-({(3R)-1-[4-oxo-4-(1-piperidinyl(butyl]-3-pyrrolidinyl}oxy)quinoline;
3-{(3R)-3-[(6-butyl-8-quinolinyl)oxy]-1-pyrrolidinyl}-N,N-diethylpropanamide;
3-{(3R)-3-[(6-butyl-8-quinolinyl)oxy]-1-pyrrolidinyl}-N-(1,1-dimethylethyl)butanamide;
4-{(3R)-3-[(6-butyl-8-quinolinyl)oxy]-1-pyrrolidinyl}-N-(1,1-dimethylethyl)butanamide;
6-butyl-8-({(3S)-1-[3-oxo-3-(1-piperidinyl)propyl]-3-pyrrolidinyl}oxy)quinoline;
6-butyl-8-({(3S)-1-[4-oxo-4-(1-piperidinyl)butyl]-3-pyrrolidinyl}oxy)quinoline;
3-{(3S)-3-[(6-butyl-8-quinolinyl)oxy]-1-pyrrolidinyl}-N,N-diethylpropanamide;
4-{(3S)-3-[(6-butyl-8-quinolinyl)oxy]-1-pyrrolidinyl}-N-(1,1-dimethylethyl)butanamide;
trans-4-({4-[(6-butyl-8-quinolinyl)oxy]-1-pipendinyl}methyl)-N-(1-methylethyl)cyclohexanecarboxamide;
trans-4-({4-[(6-butyl-8-quinolinyl)oxy]-1-pipendinyl}methyl)-N,N-diethylcyclohexane carboxamide;
6-butyl-8-[(1-{[trans-4-(1-piperidinylcarbonyl)cyclohexyl]methyl}-4-piperidinyl)oxy]quinoline;
trans-4-({4-[(6-butyl-8-quinolinyl)oxy]-1-piperidinyl}methyl)-N-propylcyclohexane carboxamide;
trans-4-({4-[(6-butyl-8-quinolinyl)oxy]-1-piperidinyl}methyl)-N-(2,2-dimethylpropyl)cyclohexane carboxamide;
trans-4-({4-[(6-butyl-8-quinolinyl)oxy]-1-piperidinyl}methyl)-N-[3-(methyloxy)propyl]cyclohexane carboxamide;
trans-4-({4-[(6-hexyl-8-quinolinyl)oxy]-1-pipendinyl}methyl)-N-(4-methylphenyl)cyclohexane carboxamide;
trans-4-({4-[(6-hexyl-8-quinolinyl)oxy]-1-pipendinyl}methyl)-N-[(4-methylphenyl)methyl]cyclohexane carboxamide;
trans-4-({4-[(6-hexyl-8-quinolinyl)oxy]-1-pipendinyl}methyl)-N-(3-methylbutyl)cyclohexane carboxamide;
6-hexyl-8-[(1-{[trans-4-(1-piperidinylcarbonyl)cyclohexyl]methyl}-4-piperidinyl)oxy]quinoline;
trans-N-(2,2-dimethylpropyl)-4-({4-[(6-hexyl-8-quinolinyl)oxy]-1-piperidinyl}methyl)cyclohexane carboxamide;
trans-N,N-diethyl-4-({4-[(6-hexyl-8-quinolinyl)oxy]-1-piperidinyl}methyl)cyclohexane carboxamide;
6-hexyl-8-[(1-{[trans-4-(1-pyrrolidinylcarbonyl)cyclohexyl]methyl}-4-piperidinyl)oxy]quinoline;
trans-4-({4-[(6-hexyl-8-quinolinyl)oxy]-1-pipendinyl}methyl)-N-propylcyclohexane carboxamide;
trans-4-({4-[(6-hexyl-8-quinolinyl)oxy]-1-pipendinyl}methyl)-N-(1-methylethyl)cyclohexane carboxamide;
6-hexyl-8-[(1-{[trans-4-(4-morpholinylcarbonyl)cyclohexyl]methyl}-4-piperidinyl)oxy]quinoline;
trans-4-({4-[(6-hexyl-8-quinolinyl)oxy]-1-piperidinyl}methyl)-N-[3-(methyloxy)propyl]cyclohexane carboxamide;
trans-4-({4-[(6-butyl-8-quinolinyl)oxy]-1-piperidinyl}methyl)-N-(4-methylphenyl)cyclohexane carboxamide;
trans-4-({4-[(6-butyl-8-quinolinyl)oxy]-1-piperidinyl}methyl)-N-[(4-methylphenyl)methyl]cyclohexane carboxamide;
trans-4-({4-[(6-butyl-8-quinolinyl)oxy]-1-pipendinyl}methyl)-N-(3-methylbutyl)cyclohexane carboxamide;
6-butyl-8-[(1-{[trans-4-(1-pyrrolidinylcarbonyl)cyclohexyl]methyl}-4-piperidinyl)oxy]quinoline;
6-butyl-8-[(1-{[trans-4-(4-morpholinylcarbonyl)cyclohexyl]methyl}-4-piperidinyl)oxy]quinoline;
N-(4-{4-[(6-butyl-8-quinolinyl)oxy]-1-piperidinyl}butyl)propanamide;
N-(4-{4-[(6-butyl-8-quinolinyl)oxy]-1-pipendinyl}butyl)butanamide;
N-(4-{4-[(6-butyl-8-quinolinyl)oxy]-1-pipendinyl}butyl)-2-methylpropanamide;
N-(4-{4-[(6-butyl-8-quinolinyl)oxy]-1-pipendinyl}butyl)-2,2-dimethylpropanamide;
N-(3-{4-[(6-butyl-8-quinolinyl)oxy]-1-piperidinyl}propyl)-2-methylpropanamide;
N-(3-{4-[(6-butyl-8-quinolinyl)oxy]-1-piperidinyl}propyl)-2,2-dimethylpropanamide;
N-(3-{4-[(6-butyl-8-quinolinyl)oxy]-1-piperidinyl}propyl)butanamide;
N-(2-{4-[(6-butyl-8-quinolinyl)oxy]-1-piperidinyl}ethyl)-2-methylpropanamide;
N-(2-{4-[(6-butyl-8-quinolinyl)oxy]-1-piperidinyl}ethyl)-2,2-dimethylpropanamide;
N-(2-{4-[(6-butyl-8-quinolinyl)oxy]-1-piperidinyl}ethyl)propanamide;
N-(2-{4-[(6-butyl-8-quinolinyl)oxy]-1-piperidinyl}ethyl)butanamide;
N-(2-{4-[(6-butyl-8-quinolinyl)oxy]-1-piperidinyl}ethyl)cyclohexane carboxamide;
N-(5-{4-[(6-butyl-8-quinolinyl)oxy]-1-piperidinyl}pentyl)-2,2-dimethylpropanamide;
N-(5-{4-[(6-butyl-8-quinolinyl)oxy]-1-piperidinyl}pentyl)propanamide;
N-(5-{4-[(6-butyl-8-quinolinyl)oxy]-1-piperidinyl}pentyl)cyclohexane carboxamide;
N-(4-{4-[(6-butyl-8-quinolinyl)oxy]-1-piperidinyl}butyl)acetamide;
N-(2-{4-[(6-butyl-8-quinolinyl)oxy]-1-piperidinyl}ethyl)-3-(methyloxy)propanamide;
N-(2-{4-[(6-butyl-8-quinolinyl)oxy]-1-piperidinyl}ethyl)-4-(methyloxy)butanamide;
N-(3-{4-[(6-butyl-8-quinolinyl)oxy]-1-piperidinyl}propyl)-3-(methyloxy)propanamide;
N-(3-{4-[(6-butyl-8-quinolinyl)oxy]-1-piperidinyl}propyl)-4-(methyloxy)butanamide;
N-(4-{4-[(6-butyl-8-quinolinyl)oxy]-1-piperidinyl}butyl)-4-(methyloxy)butanamide;
N-(4-{4-[(6-butyl-8-quinolinyl)oxy]-1-piperidinyl}butyl)-3-(methyloxy)propanamide;
N-(3-{4-[(6-butyl-8-quinolinyl)oxy]-1-piperidinyl}propyl)propanamide;
6-butyl-8-({1-[3-(3-propyl-1,2,4-oxadiazol-5-yl)propyl]-4-piperidinyl}oxy)quinoline;
N-(1,1-dimethylethyl)-4-{4-[(6-pentyl-8-quinolinyl)oxy]-1-piperidinyl}butanamide;
N,N-diethyl-4-{4-[(6-pentyl-8-quinolinyl)oxy]-1-piperidinyl}butanamide;
N-(3-{4-[(6-pentyl-8-quinolinyl)oxy]-1-piperidinyl}propyl)propanamide;
2,2-dimethyl-N-(3-{4-[(6-pentyl-8-quinolinyl)oxy]-1-piperidinyl}propyl)propanamide;
or a salt thereof.

14. A compound according to claim 1, or a pharmaceutically acceptable salt.

15-17. (canceled)

18. A composition which comprises a compound as defined in claim 1, or a pharmaceutically acceptable salt thereof, and one or more pharmaceutically acceptable carriers and/or excipients.

19-23. (canceled)

24. A method for the treatment of inflammatory and/or allergic diseases which comprises administering to a patient in need thereof an effective amount of a compound of formula (I) as defined in claim 1, or a pharmaceutically acceptable salt thereof.

25. A method according to claim 24, wherein the disease is allergic rhinitis.

26. A method according to claim 24, wherein the compound is administered intranasally.

27. A composition which comprises a compound of claim 13, or a pharmaceutically acceptable salt thereof, and one or more pharmaceutically acceptable carriers and/or excipients.

28. A method for the treatment of inflammatory and/or allergic diseases which comprises administering to a patient in need thereof an effective amount of a compound of formula (I) as defined in claim 13, or a pharmaceutically acceptable salt thereof.

29. A method according to claim 28, wherein the disease is allergic rhinitis.

30. A method according to claim 29, wherein the compound is administered intranasally.
